(19) United States
(12) Patent Application Publication
(10) Pub. No.: US 2016/0339022 A1
(43) Pub. Date: Nov. 24, 2016

(54) TREATMENT OF NEUROBLASTOMA WITH HISTONE DEACETYLASE INHIBITORS

(71) Applicant: Acetylon Pharmaceuticals Inc., Boston, MA (US)

(72) Inventors: David Lee Tamang, Watertown, MA (US); Simon S. Jones, Harvard, MA (US); Min Yang, Newton Center, MA (US)

(21) Appl. No.: 15/130,351

(22) Filed: Apr. 15, 2016

Related U.S. Application Data
(60) Provisional application No. 62/250,638, filed on Nov. 4, 2015.
(60) Provisional application No. 62/148,851, filed on Apr. 17, 2015.

(51) Int. Cl. A61K 31/505; A61K 31/496; A61K 31/5377

(52) U.S. Cl. CPC A61K 31/505; A61K 31/5377; A61K 31/496

(57) ABSTRACT

Provided herein are combinations comprising an HDAC inhibitor and retinoic acid for the treatment of neuroblastoma in a subject in need thereof. Also provided herein are methods for treating neuroblastoma in a subject in need thereof, comprising administering to the subject an effective amount of the above HDAC inhibitor or combination, comprising administering to the subject in need thereof an effective amount of the above HDAC inhibitor or combination. Also provided herein is a method for predicting whether a neuroblastoma patient will respond to treatment with a combination comprising an HDAC inhibitor and retinoic acid.

### RELATED APPLICATION

This application claims priority to U.S. Provisional Patent Application No. 62/148,851, filed Apr. 17, 2015, and U.S. Provisional Patent Application No. 62/250,638, filed Nov. 4, 2015, the contents of which are incorporated herein by reference in their entirety.

### BACKGROUND

Neuroblastoma is an extra-cranial solid cancer arising from the neural crest and is among the most common cancers in infants less than 1 year of age. Approximately one child per 100,000 is diagnosed with neuroblastoma, resulting in 650 new cases each year in the United States. Current treatment for this high-risk disease is aggressive, including chemotherapy, surgery, radiation with stem cell transplant, anti-GD2/cytokine immunotherapy, and retinoic acid. Half of the children with neuroblastoma have high risk disease and 20%-50% of those children will fail to respond adequately to current therapies, illustrating a clear unmet medical need.

### SUMMARY

Provided herein are methods of treating neuroblastoma comprising administering to a subject in need thereof a histone deacetylase inhibitor. Also provided herein is a pharmaceutical combination for treating neuroblastoma, comprising a therapeutically effective amount of a histone deacetylase (HDAC) inhibitor or a pharmaceutically acceptable salt thereof, and retinoic acid or a pharmaceutically acceptable salt thereof. In one embodiment, the retinoic acid is all-trans-retinoic acid (ATRA). In another embodiment, the HDAC inhibitor is an HDAC1/2 inhibitor. In another embodiment, the HDAC inhibitor is an HDAC1/2-specific inhibitor.

In an aspect, provided herein is a method for treating neuroblastoma in a subject in need thereof comprising administering to the subject a therapeutically effective amount of a histone deacetylase (HDAC) inhibitor or a pharmaceutically acceptable salt thereof. In yet another aspect, provided herein is a method for treating neuroblastoma in a subject in need thereof comprising administering to the subject a therapeutically effective amount of a pharmaceutical combination comprising a histone deacetylase (HDAC) inhibitor or a pharmaceutically acceptable salt thereof, and retinoic acid or a pharmaceutically acceptable salt thereof.

In embodiments of these aspects, the retinoic acid is ATRA. In another embodiment of these aspects, the HDAC inhibitor is an HDAC1/2 inhibitor. In another embodiment, the HDAC inhibitor is an HDAC1/2-specific inhibitor. In another embodiment, the subject was previously refractory to ATRA.

In another embodiment of these aspects, the HDAC inhibitor is a compound of Formula I:

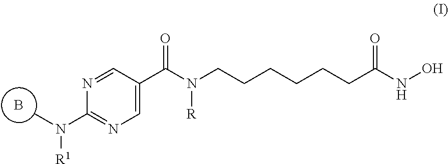

or a pharmaceutically acceptable salt thereof.

In another embodiment of these aspects, the compound of Formula I is:

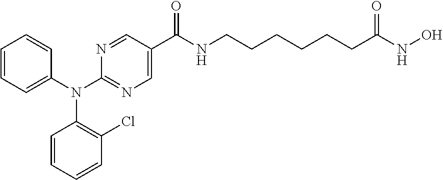 [SMILES: O=C(CCCCCCNC(=O)c1cnc(N(c2ccccc2)c2ccccc2Cl)nc1)NO]

or a pharmaceutically acceptable salt thereof.

In another embodiment of these aspects, the HDAC1/2-specific inhibitor is a compound of Formula II, or a pharmaceutically acceptable salt thereof.

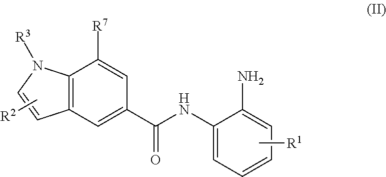

In yet another embodiment, the compound of Formula II is:

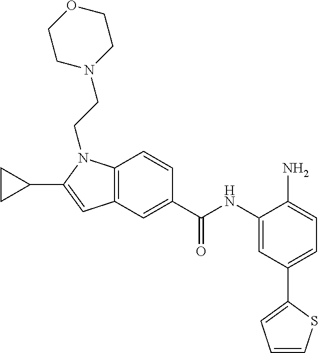 [SMILES: Nc1ccc(-c2cccs2)cc1NC(=O)c1ccc2c(c1)cc(C1CC1)n2CCN1CCOCC1]

or a pharmaceutically acceptable salt thereof.

In another embodiment of these aspects, the HDAC inhibitor is:

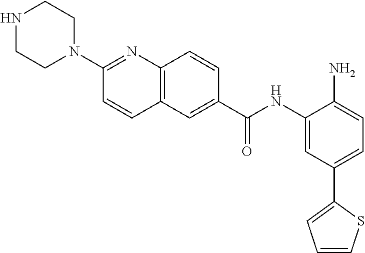 [SMILES: Nc1ccc(-c2cccs2)cc1NC(=O)c1ccc2nc(N3CCNCC3)ccc2c1]

or a pharmaceutically acceptable salt thereof.

In still another embodiment, the combination further comprises a pharmaceutically acceptable carrier.

In another aspect, provided herein is a method for predicting whether a neuroblastoma patient will respond to treatment with a pharmaceutical combination comprising an HDAC inhibitor and retinoic acid comprising the steps of:

a) administering to the patient a pharmaceutical combination comprising an HDAC inhibitor and retinoic acid;

b) taking a biological sample from the patient;

c) measuring the expression level of one or more genes selected from the group consisting of BMP4, RGS16, IER3, RGL1, SGK, CTSH, ETS1, ETS1, DUSP6, SIPA1L2, EGR1, FOS, HSPA5, NPC2, PQLC3, CFD, DHRS2, POU4F1, MYLIP, AIF1L, HMMR, SCPEP1, MERTK, LOC338758, CIB1, COL5A1, CTSL2, IFI6, CGN, CPVL, PPP2R2B, CCDC99, CYP2J2, BAMBI, HSPA1A, RN7SK, ITPR1, SPA17, ESRRG, CLDN11, ST6GALNAC3, STAT1, PPP1R3C, CRY1, RYBP, FSTL5, PRSS35, SERPINE2, HMMR, GLRX, LMO4, IL13RA2, IGSF3, NEK1, CAST, PAG1, STK3, NPTX2, CAP1, HSPA2, SDF2L1, ACO1, MAP4K2, CRYZ, DNCL1, CREG1, RHBDF2, PYGL, LRRC1, LOC730432, SERPINI1, CBR4, RAB23, VCL, ETV5, TIPARP, ALS2, SDCBP, FERMT2, TJP1, POP5, LCMT2, CEP55, PLCB1, KIAA1618, BCL2L12, PDGFD, CDC14B, CRELD2, FLJ35767, SCN9A, LOC441089, PLS1, CYP26B1, RET, RET, CRABP2, CYP26A1, ATP7A, TSPAN1, NFKBIZ, DHRS3, RARB, PLAT, VGF, PTGER2, PCDH18, ENPP2, NAV2, RARB, PLS3, CYP1B1, LOC387763, PCDH18, PDZRN3, ENPP2, RET, MMP11, TRAF3IP2, LOC375295, PRKCH, TMX4, CYP26A1, EFNB2, TMX4, PDZRN3, FNDC5, NCOA3, THBS1, LOXL4, CHRNA3, NAV2, IRF9, REPS2, FRMD6, NEDD4L, FOXC1, RARA, REPS2, ABCA1, GNG2, PDZRN3, CHRNA3, SMOC1, AKR1C3, PRMT6, ALX3, NEDD9, RND3, C10orf33, CDKN1A, ACSL3, PLS3, CRISPLD1, CRISPLD1, PCDH20, RPL26, LOC729236, JARID2, RNU6-1, HOXD1, ATP6AP2, SPRY4, REC8, FZD7, TMEM50B, RDH10, RN5S9, NPTN, G3BP2, ITGA1, NPTN, UBLCP1, IL10RB, ARMET, SH2B3, ADD3, ACSL3, RNU6-15, LOC653158, SGK1, ZFAND6, BCHE, HSD17B12, SNORA79, LIPA, G3BP1, LAMC1, CNN2, ABCB1, GLCE, FLOT1, SPRED1, VASN, XPR1, CYB5R4, FAM69A, XPR1, SC5DL, TMEM19, DNAJB11, HSP90B1, PAPSS1, FGFR1OP2, WDR1, HSD17B12, WDR44, OSTF1, SGK1, S100A10, SIPA1, SCGN, PLS1, RALB, TMC6, EXTL2, PNPLA8, YIPF1, GPR177, TRAM2, CXorf57, MYCNOS, COQ10B, PIGM, ELMOD1, DNAJB6, LOC653156, REC8, TMBIM4, TJP1, USP8, OSBPL3, CPVL, DUSP5, CADM1, SEC24D, MYADM, LOC285359, MYL12A, C3orf59, BCL6, EPB41L5, CXorf45, ZSWIM6, DCBLD2, LAMP2, HLA-B, LOC401076, TXNDC9, PCDH17, YIPF1, LOC729646, PTGR1, IGF2R, EPB41L5, LOC100129685, PAQR8, RPGR, FBLN2, GCA, GPR126, PI15, GNS, ALG13, TP53INP1, NPPA, USP38, PSMA4, C5orf32, PRKCA, SEC22B, DNAJC10, UTP14C, TULP4, HIF1A, DYNC1I1 ANKRD57, PON2, BMPR2, SLC4A8, ATP2B1, DAD1, RAB3IP, RPPH1, PRG2, PRKAR1A, ZMYM1, CLINT1, TMCO1, PDGFD, USP9X, AADACL4, BCL2L12, ALPL, LOC653079, CCDC128, HDAC1, HLA-E, INTS6, TMEM166, NDFIP2, EDEM3, FER1L4, CHUK, C10orf75, LOC389342, RNASEL, LOC100131205, TMEM205, RRBP1, ALCAM, ATG4C, MEGF9, C1orf97, STRADB, SREBF1, SUOX, RAB8B, SPRY1, ARL6IP1, C12orf34, RPAP3, LOC728782, PLEKHA6, KLF10, CD44, SNORA8, CDH24, DLK1, PTCHD1, SLC6A15, STMN4, MIAT, C16orf53, PCOLCE, TYMS, ASAM, FLJ25404, ICA1, SLC6A15, DUSP26, SH2D3C, LRFN4, CENPV, DDX17, C16orf53, CLASP2, ARMCX1, ICA1, LAMB1, CLK1, TH, P4HTM, D4S234E, MTA1, TUB, PHF17, TAGLN3, SYTL4, ARHGDIG, ABR, SNORA18, H2AFY2, ST6GAL1, DUSP8, TFAP2B, RCN1, ZNF536, F12, SCRG1, LRRTM2, GRIN1, SEZ6L2, GRM8, CENTA1, HDGF, JAM2, DDR2, MYT1, PCGF2, CNTNAP1, EML5, C1orf43, BRSK1, N4BP2L1, TCEAL7, TAGLN3, NME4, DLK1, SNHG7, MEG3, ATP1A1, LOC100131866, LOC728452, LOC441763, LOC651816, CALML4, CD320, TRAP1, ST3GAL4, LOC647251, VIM, DCN, TRERF1, SLC29A1, C2orf48, INSM2, CACNA1H, ILVBL, NELL1, LOC648210, TUBA1A, ACTG1, LOC100008588, LOC100133565, TUBB, LOC92755, LOC100133372, TUBA1C, ACTB, RTN1, LOC642817, FLJ39632, LOC91561, LOC645691, LOC100131609, PHOX2B, LOC388654, RPLP0, PHOX2B, IRF2BP2, TMEM132A, CCT7, SIX3, LOC645436, LOC648210, HMGA1, LOC148430, RPS2, LOC645385, ALDOA, LOC728698, EEF1G, LOC728643, RPLP0, SORBS2, MYCN, GUSBL1, SORBS2, RPS9, LOC729926, C1orf43, LOC100008589, GTF2IP1, ATP1A1, LOC646294, LOC391075, LOC402112, ALDOA, LOC728565, LOC646785, RPS9, TPI1, TCP1, LOC644063, APP, LOC440589, LOC284821, LOC100129553, PGAM1, LOC643357, PRMT1, PLD6, LOC647000, PRDX2, HAND2, LOC100131609, GTF2IP1, MATR3, ATF4, LOC100132528, LOC347544, LOC440589, PLCXD3, LOC728658, LOC651149, PRDX2, SNHG7, LOC729779, NCL, LOC285053, MTHFD2, SMA4, LOC441775, CAPRIN1, LOC648695, LOC648249, HIST3H2A, LOC644774, ZIC2, NPIP, SSR2, LGALS3BP, TSPO, LOC387867, NDUFA4L2, GREM1, LOC728732, SPAG9, TH, MPST, NPDC1, ACP1, ATP2C1, CASC3, LOC441506, LOC646531, PQBP1, LOC100008589, LOC100128771, B3GNT6, RNF5P1, LOC153561, NUMA1, NXPH1, RELN, SNORA67, TTC8, NFKBIA, SPTBN1, LOC100132394, GAB2, LOC652900, GLCCI1, CKAP5, LOC388707, SNRPN, SMA5, CNBP, MYT1L, LOC100128266, CD276, PHB2, HDGF2, FLJ22184, SCARB1, RBMX, MBTPS1, TMOD1, LOC441013, LOC643531, MIR1978, ATN1, FBLN1, GUSBL1, BIN1, CAMKV, LOC728658, LOC440349, HDAC9, SMA4, UNC5A, LOC390354, UNG, PRMT1, FTL, 3-Sep, ATCAY, PYCR1, RANBP1, GNG4, TAGLN2, LOC440157, CUEDC2, NFIX, TH1L, SUMO2, SORL1, DEAF1, LOC92755, CKAP4, C12orf24, TUBB4Q, LOC728139, PRRT2, LOC100130561, TACC2, MAP1B, PKMYT1, UCK2, LOC652489, IRF2BP2, EEF1D, RALY, PFKP, CCDC136, RNF165, NOMO1, TCF3, LOC401537, TNPO1, ST8SIA2, STMN2, APIP, ATP1A1, LOC649150, PKD1, LOC643300, PLOD3, SDHA, GPX7, THOC4, PRRX2, SGPP2, APEX1, PHF2, CABC1, LOC100134241, LOC732007, CCT6A, FTL, THOC3, PRR7, MCM2, C9orf86, CSNK1E, MGAT3, FEZ1, PODXL2, ENO2, LMO3, WDR5, LOC399804, PKM2, PLEKHG3, PLD6, B4GALNT4, GUSBL1, PCBP4, C12orf57, LOC651198, GAPDH, LOC402251, PALM, PCK2, ACO2, TIAL1, PTPRD, MARCKSL1, 3-Sep, PISD, PTK7, FAF1, SLC35F3, H2AFX, GNL3, FAM57B, CDK5R1, TNIP1, EEF1D, TRPC4AP, RAD51AP1, PSCD1, RELN, SIGMAR1, STXBP1, LOC643873, SKP2, HNRPK, FEZ1, HNRNPL, ADM, DBNDD2, LOC643668, NGFRAP1, FOXK1, CENTG3, NME3, EIF4A1, LOC100131735, SAC3D1, LOC100134364, TMSB10, IDH2, DPM3, PRKCZ, EIF4H, GAS6, NHP2, CNTFR, LOC440927, LOC286444, LOC100133840, TSC22D3, KIAA0195, LOC728873, BIN1, RSL1D1, N4BP2L1, NIPSNAP1, GPSM1, COLEC11, TNC, LOC100129585, NDUFV1, TPT1, ZNF423, UCKL1, MDK, TIGA1, LOC727761, FAM125B, LOC157627, SDC1, SLC10A4, SCAMP5, DAPK1, LOC389141, HRK, LOC100132060, PNMA3, DYRK2, MRPS24, LOC648927, FRZB, KLF11, LOC644237, LOC648024, TNRC4, HNRNPK, CALD1, PWWP2B, WDR45L, LOC440595, HDAC9, TRIM28, ADAR, TMEM101, PEG10, HNRNPA3, LOC100134648, LOC728411, GAPDH, GRIA4, CACNA1H, SNHG3-RCC1, EEF1A1, SLC4A2, TUBB3, PIM1, ZNRD1, ZNF536, RPL13A, DBNDD1, TXNDC5, PDZD4, SLC27A3, and RPL12 in the biological sample from the patient;

d) determining whether there is a greater than 2-fold increase in expression of one or more genes selected from the group consisting of BMP4, RGS16, IER3, RGL1, SGK, CTSH, ETS1, ETS1, DUSP6, SIPA1L2, EGR1, FOS, HSPA5, NPC2, PQLC3, CFD, DHRS2, POU4F1, MYLIP, AIF1L, HMMR, SCPEP1, MERTK, LOC338758, CIB1, COL5A1, CTSL2, IFI6, CGN, CPVL, PPP2R2B, CCDC99, CYP2J2, BAMBI, HSPA1A, RN7SK, ITPR1, SPA17, ESRRG, CLDN11, ST6GALNAC3, STAT1, PPP1R3C, CRY1, RYBP, FSTL5, PRSS35, SERPINE2, HMMR, GLRX, LMO4, IL13RA2, IGSF3, NEK1, CAST, PAG1, STK3, NPTX2, CAP1, HSPA2, SDF2L1, ACO1, MAP4K2, CRYZ, DNCL1, CREG1, RHBDF2, PYGL, LRRC1, LOC730432, SERPINI1, CBR4, RAB23, VCL, ETV5, TIPARP, ALS2, SDCBP, FERMT2, TJP1, POP5, LCMT2, CEP55, PLCB1, KIAA1618, BCL2L12, PDGFD, CDC14B, CRELD2, FLJ35767, SCN9A, LOC441089, PLS1, CYP26B1, RET, RET, CRABP2, CYP26A1, ATP7A, TSPAN1, NFKBIZ, DHRS3, RARB, PLAT, VGF, PTGER2, PCDH18, ENPP2, NAV2, RARB, PLS3, CYP1B1, LOC387763, PCDH18, PDZRN3, ENPP2, RET, MMP11, TRAF3IP2, LOC375295, PRKCH, TMX4, CYP26A1, EFNB2, TMX4, PDZRN3, FNDC5, NCOA3, THBS1, LOXL4, CHRNA3, NAV2, IRF9, REPS2, FRMD6, NEDD4L, FOXC1, RARA, REPS2, ABCA1, GNG2, PDZRN3, CHRNA3, SMOC1, AKR1C3, PRMT6, ALX3, NEDD9, RND3, C10orf33, CDKN1A, ACSL3, PLS3, CRISPLD1, CRISPLD1, PCDH20, RPL26, LOC729236, JARID2, RNU6-1, HOXD1, ATP6AP2, SPRY4, REC8, FZD7, TMEM50B, RDH10, RN5S9, NPTN, G3BP2, ITGA1, NPTN, UBLCP1, IL10RB, ARMET, SH2B3, ADD3, ACSL3, RNU6-15, LOC653158, SGK1, ZFAND6, BCHE, HSD17B12, SNORA79, LIPA, G3BP1, LAMC1, CNN2, ABCB1, GLCE, FLOT1, SPRED1, VASN, XPR1, CYB5R4, FAM69A, XPR1, SC5DL, TMEM19, DNAJB11, HSP90B1, PAPSS1, FGFR1OP2, WDR1, HSD17B12, WDR44, OSTF1, SGK1, S100A10, SIPA1, SCGN, PLS1, RALB, TMC6, EXTL2, PNPLA8, YIPF1, GPR177, TRAM2, CXorf57, MYCNOS, COQ10B, PIGM, ELMOD1, DNAJB6, LOC653156, REC8, TMBIM4, TJP1, USP8, OSBPL3, CPVL, DUSP5, CADM1, SEC24D, MYADM, LOC285359, MYL12A, C3orf59, BCL6, EPB41L5, CXorf45, ZSWIM6, DCBLD2, LAMP2, HLA-B, LOC401076, TXNDC9, PCDH17, YIPF1, LOC729646, PTGR1, IGF2R, EPB41L5, LOC100129685, PAQR8, RPGR, FBLN2, GCA, GPR126, PI15, GNS, ALG13, TP53INP1, NPPA, USP38, PSMA4, C5orf32, PRKCA, SEC22B, DNAJC10, UTP14C, TULP4, HIF1A, DYNC1I1, ANKRD57, PON2, BMPR2, SLC4A8, ATP2B1, DAD1, RAB3IP, RPPH1, PRG2, PRKAR1A, ZMYM1, CLINT1, TMCO1, PDGFD, USP9X, AADACL4, BCL2L12, ALPL, LOC653079, CCDC128, HDAC1, HLA-E, INTS6, TMEM166, NDFIP2, EDEM3, FER1L4, CHUK, C10orf75, LOC389342, RNASEL, LOC100131205, TMEM205, RRBP1, ALCAM, ATG4C, MEGF9, C1orf97, STRADB, SREBF1, SUOX, RAB8B, SPRY1, ARL6IP1, C12orf34, RPAP3, LOC728782, PLEKHA6, and KLF10, or a greater than 2-fold decrease in expression of one or more genes selected from the group consisting of CD44, SNORA8, CDH24, DLK1, PTCHD1, SLC6A15, STMN4, MIAT, C16orf53, PCOLCE, TYMS, ASAM, FLJ25404, ICA1, SLC6A15, DUSP26, SH2D3C, LRFN4, CENPV, DDX17, C16orf53, CLASP2, ARMCX1, ICA1, LAMB1, CLK1, TH, P4HTM, D4S234E, MTA1, TUB, PHF17, TAGLN3, SYTL4, ARHGDIG, ABR, SNORA18, H2AFY2, ST6GAL1, DUSP8, TFAP2B, RCN1, ZNF536, F12, SCRG1, LRRTM2, GRIN1, SEZ6L2, GRM8, CENTA1, HDGF, JAM2, DDR2, MYT1, PCGF2, CNTNAP1, EML5, C1orf43, BRSK1, N4BP2L1, TCEAL7, TAGLN3, NME4, DLK1, SNHG7, MEG3, ATP1A1, LOC100131866, LOC728452, LOC441763, LOC651816, CALML4, CD320, TRAP1, ST3GAL4, LOC647251, VIM, DCN, TRERF1, SLC29A1, C2orf48, INSM2, CACNA1H, ILVBL, NELL1, LOC648210, TUBA1A, ACTG1, LOC100008588, LOC100133565, TUBB, LOC92755, LOC100133372, TUBA1C, ACTB, RTN1, LOC642817, FLJ39632, LOC91561, LOC645691, LOC100131609, PHOX2B, LOC388654, RPLP0, PHOX2B, IRF2BP2, TMEM132A, CCT7, SIX3, LOC645436, LOC648210, HMGA1, LOC148430, RPS2, LOC645385, ALDOA, LOC728698, EEF1G, LOC728643, RPLP0, SORBS2, MYCN, GUSBL1, SORBS2, RPS9, LOC729926, C1orf43, LOC100008589, GTF2IP1, ATP1A1, LOC646294, LOC391075, LOC402112, ALDOA, LOC728565, LOC646785, RPS9, TPI1, TCP1, LOC644063, APP, LOC440589, LOC284821, LOC100129553, PGAM1, LOC643357, PRMT1, PLD6, LOC647000, PRDX2, HAND2, LOC100131609, GTF2IP1, MATR3, ATF4, LOC100132528, LOC347544, LOC440589, PLCXD3, LOC728658, LOC651149, PRDX2, SNHG7, LOC729779, NCL, LOC285053, MTHFD2, SMA4, LOC441775, CAPRIN1, LOC648695, LOC648249, HIST3H2A, LOC644774, ZIC2, NPIP, SSR2, LGALS3BP, TSPO, LOC387867, NDUFA4L2, GREM1, LOC728732, SPAG9, TH, MPST, NPDC1, ACP1, ATP2C1, CASC3, LOC441506, LOC646531, PQBP1, LOC100008589, LOC100128771, B3GNT6, RNF5P1, LOC153561, NUMA1, NXPH1, RELN, SNORA67, TTC8, NFKBIA, SPTBN1, LOC100132394, GAB2, LOC652900, GLCCI1, CKAP5, LOC388707, SNRPN, SMA5, CNBP, MYT1L, LOC100128266, CD276, PHB2, HDGF2, FLJ22184, SCARB1, RBMX, MBTPS1, TMOD1, LOC441013, LOC643531, MIR1978, ATN1, FBLN1, GUSBL1, BIN1, CAMKV, LOC728658, LOC440349, HDAC9, SMA4, UNC5A, LOC390354, UNG, PRMT1, FTL, 3-Sep, ATCAY, PYCR1, RANBP1, GNG4, TAGLN2, LOC440157, CUEDC2, NFIX, TH1L, SUMO2, SORL1, DEAF1, LOC92755, CKAP4, C12orf24, TUBB4Q, LOC728139, PRRT2, LOC100130561, TACC2, MAP1B, PKMYT1, UCK2, LOC652489, IRF2BP2, EEF1D, RALY, PFKP, CCDC136, RNF165, NOMO1, TCF3, LOC401537, TNPO1, ST8SIA2, STMN2, APIP, ATP1A1, LOC649150, PKD1, LOC643300, PLOD3, SDHA, GPX7, THOC4, PRRX2, SGPP2, APEX1, PHF2, CABC1, LOC100134241, LOC732007, CCT6A, FTL, THOC3, PRR7, MCM2, C9orf86, CSNK1E, MGAT3, FEZ1, PODXL2, ENO2, LMO3, WDR5, LOC399804, PKM2, PLEKHG3, PLD6, B4GALNT4, GUSBL1, PCBP4, C12orf57, LOC651198, GAPDH, LOC402251, PALM, PCK2, ACO2, TIAL1, PTPRD, MARCKSL1, 3-Sep, PISD, PTK7, FAF1, SLC35F3, H2AFX, GNL3, FAM57B, CDK5R1, TNIP1, EEF1D, TRPC4AP, RAD51AP1, PSCD1, RELN, SIGMAR1, STXBP1, LOC643873, SKP2, HNRPK, FEZ1, HNRNPL, ADM, DBNDD2, LOC643668, NGFRAP1, FOXK1, CENTG3, NME3, EIF4A1, LOC100131735, SAC3D1, LOC100134364, TMSB10, IDH2, DPM3, PRKCZ, EIF4H, GAS6, NHP2, CNTFR, LOC440927, LOC286444, LOC100133840, TSC22D3, KIAA0195, LOC728873, BIN1, RSL1D1, N4BP2L1, NIPSNAP1, GPSM1, COLEC11, TNC, LOC100129585, NDUFV1, TPT1, ZNF423, UCKL1, MDK, TIGA1, LOC727761, FAM125B, LOC157627, SDC1, SLC10A4, SCAMP5, DAPK1, LOC389141, HRK, LOC100132060, PNMA3, DYRK2, MRPS24, LOC648927, FRZB, KLF11, LOC644237, LOC648024, TNRC4, HNRNPK, CALD1, PWWP2B, WDR45L, LOC440595, HDAC9, TRIM28, ADAR, TMEM101, PEG10, HNRNPA3, LOC100134648, LOC728411, GAPDH, GRIA4, CACNA1H, SNHG3-RCC1, EEF1A1, SLC4A2, TUBB3, PIM1, ZNRD1, ZNF536, RPL13A, DBNDD1, TXNDC5, PDZD4, SLC27A3, and RPL12, as compared to normalized gene expression level of the gene(s), indicating that the patient will respond to the pharmaceutical combination.

In one embodiment of the method for predicting whether a neuroblastome patient will respond to treatment with an HDAC inhibitor and retinoic acid, the gene having a greater than 2-fold increase in expression has a greater than 4-fold increase in expression, and the gene is selected from the group consisting of CYP26A1, CYP26B1, DHRS3, CRABP2, RARB, PTGER2, ETS1, IER3, RET, NFKBIZ, DUSP6, CDKN1A, PCDH18, CTSH, ATP7A, HSPA5, and ACSL3.

In another aspect, provided herein is a method for treating a neuroblastoma patient with a pharmaceutical combination comprising an HDAC inhibitor and retinoic acid, wherein the neuroblastoma patient is screened for response to the pharmaceutical combination, and if it is determined that the neuroblastoma patient will respond to the pharmaceutical combination, administering a therapeutically effective amount of the pharmaceutical combination to thereby treat the patient,

wherein the screening for response to the pharmaceutical combination comprises:

a) administering to the patient a pharmaceutical combination comprising an HDAC inhibitor and retinoic acid;

b) taking a biological sample from the patient;

c) measuring the expression level of one or more genes selected from the group consisting of BMP4, RGS16, IER3, RGL1, SGK, CTSH, ETS1, ETS1, DUSP6, SIPA1L2, EGR1, FOS, HSPA5, NPC2, PQLC3, CFD, DHRS2, POU4F1, MYLIP, AIF1L, HMMR, SCPEP1, MERTK, LOC338758, CIB1, COL5A1, CTSL2, IFI6, CGN, CPVL, PPP2R2B, CCDC99, CYP2J2, BAMBI, HSPA1A, RN7SK, ITPR1, SPA17, ESRRG, CLDN11, ST6GALNAC3, STAT1, PPP1R3C, CRY1, RYBP, FSTL5, PRSS35, SERPINE2, HMMR, GLRX, LMO4, IL13RA2, IGSF3, NEK1, CAST, PAG1, STK3, NPTX2, CAP1, HSPA2, SDF2L1, ACO1, MAP4K2, CRYZ, DNCL1, CREG1, RHBDF2, PYGL, LRRC1, LOC730432, SERPINI1, CBR4, RAB23, VCL, ETV5, TIPARP, ALS2, SDCBP, FERMT2, TJP1, POP5, LCMT2, CEP55, PLCB1, KIAA1618, BCL2L12, PDGFD, CDC14B, CRELD2, FLJ35767, SCN9A, LOC441089, PLS1, CYP26B1, RET, RET, CRABP2, CYP26A1, ATP7A, TSPAN1, NFKBIZ, DHRS3, RARB, PLAT, VGF, PTGER2, PCDH18, ENPP2, NAV2, RARB, PLS3, CYP1B1, LOC387763, PCDH18, PDZRN3, ENPP2, RET, MMP11, TRAF3IP2, LOC375295, PRKCH, TMX4, CYP26A1, EFNB2, TMX4, PDZRN3, FNDC5, NCOA3, THBS1, LOXL4, CHRNA3, NAV2, IRF9, REPS2, FRMD6, NEDD4L, FOXC1, RARA, REPS2, ABCA1, GNG2, PDZRN3, CHRNA3, SMOC1, AKR1C3, PRMT6, ALX3, NEDD9, RND3, C10orf33, CDKN1A, ACSL3, PLS3, CRISPLD1, CRISPLD1, PCDH20, RPL26, LOC729236, JARID2, RNU6-1, HOXD1, ATP6AP2, SPRY4, REC8, FZD7, TMEM50B, RDH10, RN5S9, NPTN, G3BP2, ITGA1, NPTN, UBLCP1, IL10RB, ARMET, SH2B3, ADD3, ACSL3, RNU6-15, LOC653158, SGK1, ZFAND6, BCHE, HSD17B12, SNORA79, LIPA, G3BP1, LAMC1, CNN2, ABCB1, GLCE, FLOT1, SPRED1, VASN, XPR1, CYB5R4, FAM69A, XPR1, SC5DL, TMEM19, DNAJB11, HSP90B1, PAPSS1, FGFR1OP2, WDR1, HSD17B12, WDR44, OSTF1, SGK1, S100A10, SIPA1, SCGN, PLS1, RALB, TMC6, EXTL2, PNPLA8, YIPF1, GPR177, TRAM2, CXorf57, MYCNOS, COQ10B, PIGM, ELMOD1, DNAJB6, LOC653156, REC8, TMBIM4, TJP1, USP8, OSBPL3, CPVL, DUSP5, CADM1, SEC24D, MYADM, LOC285359, MYL12A, C3orf59, BCL6, EPB41L5, CXorf45, ZSWIM6, DCBLD2, LAMP2, HLA-B, LOC401076, TXNDC9, PCDH17, YIPF1, LOC729646, PTGR1, IGF2R, EPB41L5, LOC100129685, PAQR8, RPGR, FBLN2, GCA, GPR126, PI15, GNS, ALG13, TP53INP1, NPPA, USP38, PSMA4, C5orf32, PRKCA, SEC22B, DNAJC10, UTP14C, TULP4, HIF1A, DYNC1I1, ANKRD57, PON2, BMPR2, SLC4A8, ATP2B1, DAD1, RAB3IP, RPPH1, PRG2, PRKAR1A, ZMYM1, CLINT1, TMCO1, PDGFD, USP9X, AADACL4, BCL2L12, ALPL, LOC653079, CCDC128, HDAC1, HLA-E, INTS6, TMEM166, NDFIP2, EDEM3, FER1L4, CHUK, C10orf75, LOC389342, RNASEL, LOC100131205, TMEM205, RRBP1, ALCAM, ATG4C, MEGF9, C1orf97, STRADB, SREBF1, SUOX, RAB8B, SPRY1, ARL6IP1, C12orf34, RPAP3, LOC728782, PLEKHA6, KLF10, CD44, SNORA8, CDH24, DLK1, PTCHD1, SLC6A15, STMN4, MIAT, C16orf53, PCOLCE, TYMS, ASAM, FLJ25404, ICA1, SLC6A15, DUSP26, SH2D3C, LRFN4, CENPV, DDX17, C16orf53, CLASP2, ARMCX1, ICA1, LAMB1, CLK1, TH, P4HTM, D4S234E, MTA1, TUB, PHF17, TAGLN3, SYTL4, ARHGDIG, ABR, SNORA18, H2AFY2, ST6GAL1, DUSP8, TFAP2B, RCN1, ZNF536, F12, SCRG1, LRRTM2, GRIN1, SEZ6L2, GRM8, CENTA1, HDGF, JAM2, DDR2, MYT1, PCGF2, CNTNAP1, EML5, C1orf43, BRSK1, N4BP2L1, TCEAL7, TAGLN3, NME4, DLK1, SNHG7, MEG3, ATP1A1, LOC100131866, LOC728452, LOC441763, LOC651816, CALML4, CD320, TRAP1, ST3GAL4, LOC647251, VIM, DCN, TRERF1, SLC29A1, C2orf48, INSM2, CACNA1H, ILVBL, NELL1, LOC648210, TUBA1A, ACTG1, LOC100008588, LOC100133565, TUBB, LOC92755, LOC100133372, TUBA1C, ACTB, RTN1, LOC642817, FLJ39632, LOC91561, LOC645691, LOC100131609, PHOX2B, LOC388654, RPLP0, PHOX2B, IRF2BP2, TMEM132A, CCT7, SIX3, LOC645436, LOC648210, HMGA1, LOC148430, RPS2, LOC645385, ALDOA, LOC728698, EEF1G, LOC728643, RPLP0, SORBS2, MYCN, GUSBL1, SORBS2, RPS9, LOC729926, C1orf43, LOC100008589, GTF2IP1, ATP1A1, LOC646294, LOC391075, LOC402112, ALDOA, LOC728565, LOC646785, RPS9, TPI1, TCP1, LOC644063, APP, LOC440589, LOC284821, LOC100129553, PGAM1, LOC643357, PRMT1, PLD6, LOC647000, PRDX2, HAND2, LOC100131609, GTF2IP1, MATR3, ATF4, LOC100132528, LOC347544, LOC440589, PLCXD3, LOC728658, LOC651149, PRDX2, SNHG7, LOC729779, NCL, LOC285053, MTHFD2, SMA4, LOC441775, CAPRIN1, LOC648695, LOC648249, HIST3H2A, LOC644774, ZIC2, NPIP, SSR2, LGALS3BP, TSPO, LOC387867, NDUFA4L2, GREM1, LOC728732, SPAG9, TH, MPST, NPDC1, ACP1, ATP2C1, CASC3, LOC441506, LOC646531, PQBP1, LOC100008589, LOC100128771, B3GNT6, RNF5P1, LOC153561, NUMA1, NXPH1, RELN, SNORA67, TTC8, NFKBIA, SPTBN1, LOC100132394, GAB2, LOC652900, GLCCI1, CKAP5, LOC388707, SNRPN, SMA5, CNBP, MYT1L, LOC100128266, CD276, PHB2, HDGF2, FLJ22184, SCARB1, RBMX, MBTPS1, TMOD1, LOC441013, LOC643531, MIR1978, ATN1, FBLN1, GUSBL1, BIN1, CAMKV, LOC728658, LOC440349, HDAC9, SMA4, UNC5A, LOC390354, UNG, PRMT1, FTL, 3-Sep, ATCAY, PYCR1, RANBP1, GNG4, TAGLN2, LOC440157, CUEDC2, NFIX, TH1L, SUMO2, SORL1, DEAF1, LOC92755, CKAP4, C12orf24, TUBB4Q, LOC728139, PRRT2, LOC100130561, TACC2, MAP1B, PKMYT1, UCK2, LOC652489, IRF2BP2, EEF1D, RALY, PFKP, CCDC136, RNF165, NOMO1, TCF3, LOC401537, TNPO1, ST8SIA2, STMN2, APIP, ATP1A1, LOC649150, PKD1, LOC643300, PLOD3, SDHA, GPX7, THOC4, PRRX2, SGPP2, APEX1, PHF2, CABC1, LOC100134241, LOC732007, CCT6A, FTL, THOC3, PRR7, MCM2, C9orf86, CSNK1E, MGAT3, FEZ1, PODXL2, ENO2, LMO3, WDR5, LOC399804, PKM2, PLEKHG3, PLD6, B4GALNT4, GUSBL1, PCBP4, C12orf57, LOC651198, GAPDH, LOC402251, PALM, PCK2, ACO2, TIAL1, PTPRD, MARCKSL1, 3-Sep, PISD, PTK7, FAF1, SLC35F3, H2AFX, GNL3, FAM57B, CDK5R1, TNIP1, EEF1D, TRPC4AP, RAD51AP1, PSCD1, RELN, SIGMAR1, STXBP1, LOC643873, SKP2, HNRPK, FEZ1, HNRNPL, ADM, DBNDD2, LOC643668, NGFRAP1, FOXK1, CENTG3, NME3, EIF4A1, LOC100131735, SAC3D1, LOC100134364, TMSB10, IDH2, DPM3, PRKCZ, EIF4H, GAS6, NHP2, CNTFR, LOC440927, LOC286444, LOC100133840, TSC22D3, KIAA0195, LOC728873, BIN1, RSL1D1, N4BP2L1, NIPSNAP1, GPSM1, COLEC11, TNC, LOC100129585, NDUFV1, TPT1, ZNF423, UCKL1, MDK, TIGA1, LOC727761, FAM125B, LOC157627, SDC1, SLC10A4, SCAMP5, DAPK1, LOC389141, HRK, LOC100132060, PNMA3, DYRK2, MRPS24, LOC648927, FRZB, KLF11, LOC644237, LOC648024, TNRC4, HNRNPK, CALD1, PWWP2B, WDR45L, LOC440595, HDAC9, TRIM28, ADAR, TMEM101, PEG10, HNRNPA3, LOC100134648, LOC728411, GAPDH, GRIA4, CACNA1H, SNHG3-RCC1, EEF1A1, SLC4A2, TUBB3, PIM1, ZNRD1, ZNF536, RPL13A, DBNDD1, TXNDC5, PDZD4, SLC27A3, and RPL12 in the biological sample from the patient;

d) determining whether there is a greater than 2-fold increase in expression of one or more genes selected from the group consisting of BMP4, RGS16, IER3, RGL1, SGK, CTSH, ETS1, ETS1, DUSP6, SIPA1L2, EGR1, FOS, HSPA5, NPC2, PQLC3, CFD, DHRS2, POU4F1, MYLIP, AIF1L, HMMR, SCPEP1, MERTK, LOC338758, CIB1, COL5A1, CTSL2, IFI6, CGN, CPVL, PPP2R2B, CCDC99, CYP2J2, BAMBI, HSPA1A, RN7SK, ITPR1, SPA17, ESRRG, CLDN11, ST6GALNAC3, STAT1, PPP1R3C, CRY1, RYBP, FSTL5, PRSS35, SERPINE2, HMMR, GLRX, LMO4, IL13RA2, IGSF3, NEK1, CAST, PAG1, STK3, NPTX2, CAP1, HSPA2, SDF2L1, ACO1, MAP4K2, CRYZ, DNCL1, CREG1, RHBDF2, PYGL, LRRC1, LOC730432, SERPINI1, CBR4, RAB23, VCL, ETV5, TIPARP, ALS2, SDCBP, FERMT2, TJP1, POP5, LCMT2, CEP55, PLCB1, KIAA1618, BCL2L12, PDGFD, CDC14B, CRELD2, FLJ35767, SCN9A, LOC441089, PLS1, CYP26B1, RET, RET, CRABP2, CYP26A1, ATP7A, TSPAN1, NFKBIZ, DHRS3, RARB, PLAT, VGF, PTGER2, PCDH18, ENPP2, NAV2, RARB, PLS3, CYP1B1, LOC387763, PCDH18, PDZRN3, ENPP2, RET, MMP11, TRAF3IP2, LOC375295, PRKCH, TMX4, CYP26A1, EFNB2, TMX4, PDZRN3, FNDC5, NCOA3, THBS1, LOXL4, CHRNA3, NAV2, IRF9, REPS2, FRMD6, NEDD4L, FOXC1, RARA, REPS2, ABCA1, GNG2, PDZRN3, CHRNA3, SMOC1, AKR1C3, PRMT6, ALX3, NEDD9, RND3, C10orf33, CDKN1A, ACSL3, PLS3, CRISPLD1, CRISPLD1, PCDH20, RPL26, LOC729236, JARID2, RNU6-1, HOXD1, ATP6AP2, SPRY4, REC8, FZD7, TMEM50B, RDH10, RN5S9, NPTN, G3BP2, ITGA1, NPTN, UBLCP1, IL10RB, ARMET, SH2B3, ADD3, ACSL3, RNU6-15, LOC653158, SGK1, ZFAND6, BCHE, HSD17B12, SNORA79, LIPA, G3BP1, LAMC1, CNN2, ABCB1, GLCE, FLOT1, SPRED1, VASN, XPR1, CYB5R4, FAM69A, XPR1, SC5DL, TMEM19, DNAJB11, HSP90B1, PAPSS1, FGFR1OP2, WDR1, HSD17B12, WDR44, OSTF1, SGK1, S100A10, SIPA1, SCGN, PLS1, RALB, TMC6, EXTL2, PNPLA8, YIPF1, GPR177, TRAM2, CXorf57, MYCNOS, COQ10B, PIGM, ELMOD1, DNAJB6, LOC653156, REC8, TMBIM4, TJP1, USP8, OSBPL3, CPVL, DUSP5, CADM1, SEC24D, MYADM, LOC285359, MYL12A, C3orf59, BCL6, EPB41L5, CXorf45, ZSWIM6, DCBLD2, LAMP2, HLA-B, LOC401076, TXNDC9, PCDH17, YIPF1, LOC729646, PTGR1, IGF2R, EPB41L5, LOC100129685, PAQR8, RPGR, FBLN2, GCA, GPR126, PI15, GNS, ALG13, TP53INP1, NPPA, USP38, PSMA4, C5orf32, PRKCA, SEC22B, DNAJC10, UTP14C, TULP4, HIF1A, DYNC1I1, ANKRD57, PON2, BMPR2, SLC4A8, ATP2B1, DAD1, RAB3IP, RPPH1, PRG2, PRKAR1A, ZMYM1, CLINT1, TMCO1, PDGFD, USP9X, AADACL4, BCL2L12, ALPL, LOC653079, CCDC128, HDAC1, HLA-E, INTS6, TMEM166, NDFIP2, EDEM3, FER1L4, CHUK, C10orf75, LOC389342, RNASEL, LOC100131205, TMEM205, RRBP1, ALCAM, ATG4C, MEGF9, C1orf97, STRADB, SREBF1, SUOX, RAB8B, SPRY1, ARL6IP1, C12orf34, RPAP3, LOC728782, PLEKHA6, and KLF10, or a greater than 2-fold decrease in expression of one or more genes selected from the group consisting of CD44, SNORA8, CDH24, DLK1, PTCHD1, SLC6A15, STMN4, MIAT, C16orf53, PCOLCE, TYMS, ASAM, FLJ25404, ICA1, SLC6A15, DUSP26, SH2D3C, LRFN4, CENPV, DDX17, C16orf53, CLASP2, ARMCX1, ICA1, LAMB1, CLK1, TH, P4HTM, D4S234E, MTA1, TUB, PHF17, TAGLN3, SYTL4, ARHGDIG, ABR, SNORA18, H2AFY2, ST6GAL1, DUSP8, TFAP2B, RCN1, ZNF536, F12, SCRG1, LRRTM2, GRIN1, SEZ6L2, GRM8, CENTA1, HDGF, JAM2, DDR2, MYT1, PCGF2, CNTNAP1, EML5, C1orf43, BRSK1, N4BP2L1, TCEAL7, TAGLN3, NME4, DLK1, SNHG7, MEG3, ATP1A1, LOC100131866, LOC728452, LOC441763, LOC651816, CALML4, CD320, TRAP1, ST3GAL4, LOC647251, VIM, DCN, TRERF1, SLC29A1, C2orf48, INSM2, CACNA1H, ILVBL, NELL1, LOC648210, TUBA1A, ACTG1, LOC100008588, LOC100133565, TUBB, LOC92755, LOC100133372, TUBA1C, ACTB, RTN1, LOC642817, FLJ39632, LOC91561, LOC645691, LOC100131609, PHOX2B, LOC388654, RPLP0, PHOX2B, IRF2BP2, TMEM132A, CCT7, SIX3, LOC645436, LOC648210, HMGA1, LOC148430, RPS2, LOC645385, ALDOA, LOC728698, EEF1G, LOC728643, RPLP0, SORBS2, MYCN, GUSBL1, SORBS2, RPS9, LOC729926, C1orf43, LOC100008589, GTF2IP1, ATP1A1, LOC646294, LOC391075, LOC402112, ALDOA, LOC728565, LOC646785, RPS9, TPI1, TCP1, LOC644063, APP, LOC440589, LOC284821, LOC100129553, PGAM1, LOC643357, PRMT1, PLD6, LOC647000, PRDX2, HAND2, LOC100131609, GTF2IP1, MATR3, ATF4, LOC100132528, LOC347544, LOC440589, PLCXD3, LOC728658, LOC651149, PRDX2, SNHG7, LOC729779, NCL, LOC285053, MTHFD2, SMA4, LOC441775, CAPRIN1, LOC648695, LOC648249, HIST3H2A, LOC644774, ZIC2, NPIP, SSR2, LGALS3BP, TSPO, LOC387867, NDUFA4L2, GREM1, LOC728732, SPAG9, TH, MPST, NPDC1, ACP1, ATP2C1, CASC3, LOC441506, LOC646531, PQBP1, LOC100008589, LOC100128771, B3GNT6, RNF5P1, LOC153561, NUMA1, NXPH1, RELN, SNORA67, TTC8, NFKBIA, SPTBN1, LOC100132394, GAB2, LOC652900, GLCCI1, CKAP5, LOC388707, SNRPN, SMA5, CNBP, MYT1L, LOC100128266, CD276, PHB2, HDGF2, FLJ22184, SCARB1, RBMX, MBTPS1, TMOD1, LOC441013, LOC643531, MIR1978, ATN1, FBLN1, GUSBL1, BIN1, CAMKV, LOC728658, LOC440349, HDAC9, SMA4, UNC5A, LOC390354, UNG, PRMT1, FTL, 3-Sep, ATCAY, PYCR1, RANBP1, GNG4, TAGLN2, LOC440157, CUEDC2, NFIX, TH1L, SUMO2, SORL1, DEAF1, LOC92755, CKAP4, C12orf24, TUBB4Q, LOC728139, PRRT2, LOC100130561, TACC2, MAP1B, PKMYT1, UCK2, LOC652489, IRF2BP2, EEF1D, RALY, PFKP, CCDC136, RNF165, NOMO1, TCF3, LOC401537, TNPO1, ST8SIA2, STMN2, APIP, ATP1A1, LOC649150, PKD1, LOC643300, PLOD3, SDHA, GPX7, THOC4, PRRX2, SGPP2, APEX1, PHF2, CABC1, LOC100134241, LOC732007, CCT6A, FTL, THOC3, PRR7, MCM2, C9orf86, CSNK1E, MGAT3, FEZ1, PODXL2, ENO2, LMO3, WDR5, LOC399804, PKM2, PLEKHG3, PLD6, B4GALNT4, GUSBL1, PCBP4, C12orf57, LOC651198, GAPDH, LOC402251, PALM, PCK2, ACO2, TIAL1, PTPRD, MARCKSL1, 3-Sep, PISD, PTK7, FAF1, SLC35F3, H2AFX, GNL3, FAM57B, CDK5R1, TNIP1, EEF1D, TRPC4AP, RAD51AP1, PSCD1, RELN, SIGMAR1, STXBP1, LOC643873, SKP2, HNRPK, FEZ1, HNRNPL, ADM, DBNDD2, LOC643668, NGFRAP1, FOXK1, CENTG3, NME3, EIF4A1, LOC100131735, SAC3D1, LOC100134364, TMSB10, IDH2, DPM3, PRKCZ, EIF4H, GAS6, NHP2, CNTFR, LOC440927, LOC286444, LOC100133840, TSC22D3, KIAA0195, LOC728873, BIN1, RSL1D1, N4BP2L1, NIPSNAP1, GPSM1, COLEC11, TNC, LOC100129585, NDUFV1, TPT1, ZNF423, UCKL1, MDK, TIGA1, LOC727761, FAM125B, LOC157627, SDC1, SLC10A4, SCAMP5, DAPK1, LOC389141, HRK, LOC100132060, PNMA3, DYRK2, MRPS24, LOC648927, FRZB, KLF11, LOC644237, LOC648024, TNRC4, HNRNPK, CALD1, PWWP2B, WDR45L, LOC440595, HDAC9, TRIM28, ADAR, TMEM101, PEG10, HNRNPA3, LOC100134648, LOC728411, GAPDH, GRIA4, CACNA1H, SNHG3-RCC1, EEF1A1, SLC4A2, TUBB3, PIM1, ZNRD1, ZNF536, RPL13A, DBNDD1, TXNDC5, PDZD4, SLC27A3, and RPL12, as compared to normalized gene expression level of the gene(s), indicating that the patient will respond to the pharmaceutical combination.

### BRIEF DESCRIPTION OF THE FIGURES

FIG. 1A shows that HDAC1/2i induce gene expression consistent with differentiation and that in combination with ATRA, the differentiation effect is enhanced; data for Compound A and ATRA on SK-N-BE(2) cells shown.

FIG. 1B shows that HDAC1/2i induce gene expression consistent with differentiation and that in combination with ATRA, the differentiation effect is enhanced;

data for Compound B and ATRA on SK-N-BE(2) cells shown.

FIG. 1C shows that HDAC1/2i induce gene expression consistent with differentiation and that in combination with ATRA, the differentiation effect is enhanced; inhibition of HDAC2 activity in live cells with potency in the 0.5-3 μM range shown.

FIG. 1D shows that Compound B induces histone acetylation in neuroblastoma cells as shown by Western blot at 24 hours.

FIG. 2A shows that the addition of 1 μM or 3 μM ATRA, which has potent differentiation activity, has little effect on HDAC1/2i mediated toxicity, indicating that HDAC1/2i toxicity is independent of ATRA, and occurs at concentrations greater than those needed for differentiation.

FIG. 2B shows that Caspase 3/7 activity is increased at concentrations ≧2 μM Compound B, and that the increased activity is independent of the presence of ATRA, indicating that HDAC1/2i toxicity is independent of ATRA, and occurs at concentrations greater than those needed for differentiation.

FIG. 3A shows that differentiation is induced by non-cytotoxic concentrations of Compound B; data for Compound B on SK-N-BE(2) cells shown.

FIG. 3B shows that differentiation is induced by non-cytotoxic concentrations of Compound A; data for Compound A on SK-N-BE(2) cells shown.

FIG. 4A shows that Compound B combines with ATRA to enhance suppression of neuroblastoma proliferation; data for 1 μM Compound B shown.

FIG. 4B shows that Compound B combines with ATRA to enhance suppression of neuroblastoma proliferation; data for 3 μM Compound B shown.

FIG. 5A shows that Compound A combines with ATRA to enhance suppression of neuroblastoma proliferation; data for 0.75 μM Compound A shown.

FIG. 5B shows that Compound A combines with ATRA to enhance suppression of neuroblastoma proliferation; data for 2 μM Compound A shown.

FIG. 6A shows that Compound B induced increased expression of the master cell cycle regulator p21 as a single agent and in a dose-dependent manner; Compound B enhances ATRA-mediated cell cycle arrest.

FIG. 6B shows that single-agent ATRA reduces s-phase frequency and increases the sub-G1 population, and that this effect is enhanced by Compound B in a dose dependent manner; Compound B enhances ATRA-mediated cell cycle arrest.

FIG. 6C shows the population of each stage of the cell cycle at various concentrations of Compound B and ATRA alone, or in combination; Compound B enhances ATRA-mediated cell cycle arrest.

FIG. 7 shows that Compound B combines with ATRA to enhance morphology changes consistent with differentiation.

FIG. 8 shows that Compound B combines with ATRA to enhance NF-M staining.

FIG. 9 shows that Compound B combines with ATRA to reduce neuroblastoma outgrowth.

FIG. 10 shows that Compound B (HDAC1/2i) shows stronger combination effect with ATRA than Compound C (HDAC3i; N-(2-amino-4-fluorophenyl)-8-cyclopropyl-7-(piperazin-1-yl)quinoline-3-carboxamide) on cell proliferation and dendrite outgrowth.

FIG. 11 shows that the combination of Compound B with ATRA completely blocks cluster formation of NB at concentrations lower than Compound C (HDAC3i).

FIG. 12A shows gene expression changes and overlaps of SK-N-BE(2) cells treated with 3 μM Compound B, 1 μM ATRA, or a combination of both at 37° C. over 2 hours when compared to a solvent (DMSO) control.

FIG. 12B shows gene expression changes and overlaps of SK-N-BE(2) cells treated with 3 μM Compound B, 1 μM ATRA, or a combination of both at 37° C. over 48 hours when compared to a solvent (DMSO) control.

FIG. 13 shows that ATRA alters binding positions of retinoic acid receptor (RAR) to chromatin, and this altered binding is enhanced by Compound B.

FIG. 14A shows a model for HDACi enhancement of ATRA-mediated differentiation.

FIG. 14B shows a simplified model for HDACi enhancement of ATRA-mediated differentiation.

FIG. 15 shows that HDAC1/2 inhibitors in combination with ATRA disrupt the Wnt signaling pathway.

FIG. 16 shows that Retinoic Acid-activated AKT is reduced by HDAC1/2i.

FIG. 17 shows the modulation of proteins involved in cell-cycle progression by HDAC1/2i.

FIG. 18 shows a diagram of pathways that were consistently enriched or suppressed by HDAC1/2 inhibition.

FIG. 19 shows that the combination of an HDAC1/2i (compound with retinoic acid enhanced cleavage of caspase 3 and 9 and PARP, consistent with apoptotic death.

FIG. 20 shows that pro-E2F signaling proteins CDK4 and 6 were decreased in the combination while the inhibitory protein p21 was increased.

FIG. 21 shows that treatment with HDAC1/2i+RA enhanced RA-induced expression of the RARβ gene.

FIG. 22 shows that treatment with HDAC1/2i+RA increased RARβ protein levels.

FIG. 23 shows that RAR binding to the RARβ gene promoter was enhanced in a combination setting (Compound B+ATRA) relative to either single agent alone.

FIG. 24 shows that Cyp26b1, a protein induced by RA and negative regulator of RAR signaling, was decreased in a combination setting (Compound B+ATRA) as measured by gene expression and protein levels.

FIG. 25 shows that DHRS3, a protein induced by RA and negative regulator of RAR signaling, was decreased in a combination (Compound B+ATRA) setting as measured by gene expression and protein levels.

FIG. 26 shows the HDAC1/2 inhibitor Compound E combines with RA to slow neuroblastoma tumor growth in vivo. (Error bars illustrate the standard error of the mean).

FIG. 27A shows that ATRA has a cytotoxic effect on neuroblastoma cells as a single agent.

FIG. 27B shows that Compound B has a cytotoxic effect on neuroblastoma cells as a single agent.

FIG. 27C shows the combination index (CI) values plotted against the fraction affected (Fa), demonstrating synergistic combination (CI values less than 1) across a wide range of Fa values.

FIG. 27D shows the combination matrix of ATRA with Compound B and illustrates the fraction of cells affected (dead cells, Fa value) and combinations where the CI value was less than 0.7 (underlined), suggesting potent synergy.

FIG. 28A shows that ATRA has a cytotoxic effect on neuroblastoma cells as a single agent.

FIG. 28B shows that Compound A has a cytotoxic effect on neuroblastoma cells as a single agent.

FIG. 28C shows the combination index (CI) values plotted against the fraction affected (Fa), demonstrating synergistic combination (CI values less than 1) across a wide range of Fa values.

FIG. 28D shows the combination matrix of ATRA with Compound A and illustrates the fraction of cells affected (dead cells, Fa value) and combinations where the CI value was less than 0.7 (underlined), suggesting potent synergy.

### DETAILED DESCRIPTION

Provided herein are methods of treating neuroblastoma in a subject in need thereof, comprising administering to the subject an HDAC inhibitor. Also provided herein are combinations comprising an HDAC inhibitor and retinoic acid, for the treatment of neuroblastoma in a subject in need thereof. Also provided herein are methods for treating neuroblastoma in a subject in need thereof, comprising administering to the subject an effective amount of the above combination comprising an HDAC inhibitor and retinoic acid. Also provided herein is a method for predicting whether a neuroblastoma patient will respond to treatment with a combination comprising an HDAC inhibitor and retinoic acid.

### DEFINITIONS

Listed below are definitions of various terms used to describe this invention. These definitions apply to the terms as they are used throughout this specification and claims, unless otherwise limited in specific instances, either individually or as part of a larger group.

The term “about” generally indicates a possible variation of no more than 10%, 5%, or 1% of a value. For example, “about 25 mg/kg” will generally indicate, in its broadest sense, a value of 22.5-27.5 mg/kg, i.e., 25±2.5 mg/kg.

The term “alkyl” refers to saturated, straight- or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a C_{X} chain means an alkyl chain containing x carbon atoms.

The term “cycloalkyl” denotes a monovalent group derived from a monocyclic or polycyclic saturated carbocyclic ring compound. Examples of C₃₋₈-cycloalkyl include, but not limited to, cyclopropyl, cyclobutyl, cyclopentyl, cyclohexyl, cyclopentyl and cyclooctyl; and examples of C₃₋₁₂-cycloalkyl include, but are not limited to, cyclopropyl, cyclobutyl, cyclopentyl, cyclohexyl, bicyclo [2.2.1] heptyl, and bicyclo [2.2.2] octyl.

The term “heterocycloalkyl” refers to a cycloalkyl ring system, as described herein, of which one ring atom is selected from S, O, N and Si; zero, one or two ring atoms are additional heteroatoms independently selected from S, O, N and Si; and the remaining ring atoms are carbon.

The term “aryl” refers to a mono- or poly-cyclic carbocyclic ring system having one or more aromatic rings, fused or non-fused, including, but not limited to, phenyl, naphthyl, tetrahydronaphthyl, indanyl, idenyl and the like.

The term “heteroaryl” refers to a mono- or poly-cyclic (e.g., bi-, or tri-cyclic or more) fused or non-fused, moieties or ring system having at least one aromatic ring, having from five to ten ring atoms of which one ring atom is selected from S, O, N and Si; zero, one or two ring atoms are additional heteroatoms independently selected from S, O, N and Si; and the remaining ring atoms are carbon. Heteroaryl includes, but is not limited to pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “halo” refers to a halogen, such as fluorine, chlorine, bromine, and iodine.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

The term “HDAC1/2i” or “HDAC1/2 inhibitor” means that the compound binds to HDAC1 and HDAC2.

The term “HDAC1/2-specific” means that the compound binds to HDAC1 and DAC2 to a substantially greater extent, such as 5×, 10×, 15×, 20× greater or more, than to any other type of HDAC enzyme, such as HDAC3 or HDAC6. That is, the compound is selective for HDAC1 and HDAC2 over any other type of HDAC enzyme. For example, a compound that binds to HDAC1 and HDAC2 with an IC₅₀ of 10 nM and to HDAC3 with an IC₅₀ of 50 nM is HDAC1/2-specific. On the other hand, a compound that binds to HDAC1 and HDAC2 with an IC₅₀ of 50 nM and to HDAC3 with an IC₅₀ of 60 nM is not HDAC1/2-specific.

The term “combination” refers to two or more therapeutic agents to treat a therapeutic condition or disorder described in the present disclosure. Such combination of therapeutic agents can be in the form of a single pill, capsule, or intravenous solution. However, the term “combination” also encompasses the situation when the two or more therapeutic agents are in separate pills, capsules, or intravenous solutions. Likewise, the term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, or in separate containers (e.g., capsules) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein. Further, in an embodiment, the two or more therapeutic agents may be administered independently, at the same time or separately within time intervals, especially where these time intervals allow that the combination partners show a cooperative, e.g., synergistic, effect.

The term “neuroblastoma” encompasses all kinds of neuroblastomas and refers to a sarcoma of nervous system origin, composed chiefly of neuroblasts and affecting mostly infants and children up to 10 years of age. Most neuroblastomas arise in the autonomic nervous system (sympathico-blasroma) or in the adrenal medulla. Specifically, all stages of a neuroblastoma are comprised by the term. Staging is carried out, preferably, according to the International Neuroblastoma Staging System (INS S) (Brodeur 1993, J Clin Oncol 11: 1466-1477). In principle, this surgical-based staging distinguishes five basic stages of neuroblastoma: Stage I: Localized tumour confined to the area of origin. Complete gross resection with or without microscopic residual disease; identifiable ipsilateral and contralateral lymph node negative for tumour. Stage II: Unilateral tumour with incomplete gross resection; identifiable ipsilateral and contralateral lymph node negative for tumour (stage II a), with ipsilateral lymph node positive for tumour, identifiable contralateral lymph node negative for tumour (stage II b). Stage III: Tumour infiltrating across the midline with or without regional lymph node involvement; or unilateral tumour with contralateral lymph node involvement or midline tumour with bilateral lymph node involvement. Stage IV: Dissemination of tumour to distant lymph nodes, bone marrow, liver, or other organs except as defined in stage IVS. Stage IVS: Localized primary tumour as defined for stage 1 or 2 with dissemination limited to liver, skin, and bone marrow (<10% of nucleated marrow cells are tumor cells).

The term “inhibitor” is synonymous with the term antagonist.

### Histone Deacetylase (HDAC) Inhibitors

Provided herein are methods for treating neuroblastoma in a subject in need thereof. Also provided herein are pharmaceutical combinations for the treatment of neuroblastoma in a subject in need thereof.

The methods and combinations of the invention comprise an HDAC inhibitor. The HDAC inhibitor can be any HDAC inhibitor. Thus, the HDAC inhibitor can be specific or non-specific to a particular type of histone deacetylase enzyme. Preferably, the HDAC inhibitor is an HDAC1/2 inhibitor. More preferably, the HDAC inhibitor is an HDAC1/2-specific inhibitor.

In some embodiments, the HDAC inhibitor is a compound of Formula I:

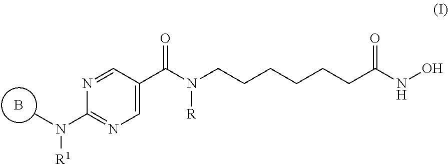

or a pharmaceutically acceptable salt thereof,
wherein,
ring B is aryl or heteroaryl;
R¹ is an aryl or heteroaryl, each of which may be optionally substituted by OH, halo, or C₁₋₆-alkyl; and
R is H or C₁₋₆-alkyl.

In one embodiment, R¹ is an aryl or heteroaryl, each of which is substituted by halo.

Representative compounds of Formula I include, but are not limited to:

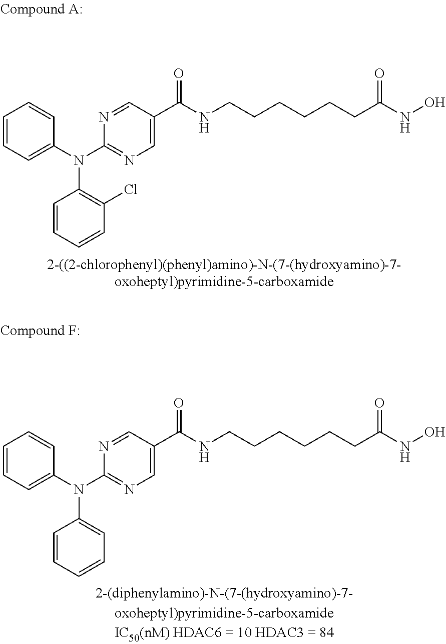 [SMILES: O=C(CCCCCCNC(=O)c1cnc(N(c2ccccc2)c2ccccc2Cl)nc1)NO ; O=C(CCCCCCNC(=O)c1cnc(N(c2ccccc2)c2ccccc2)nc1)NO]

or pharmaceutically acceptable salts thereof.

The preparation and properties of HDAC inhibitors according to Formula I are provided in International Patent Application No. PCT/US2011/021982, the entire contents of which are incorporated herein by reference. The HDAC inhibitory profile of Compound A is found in Example 3, Table 1.

In another embodiment, the HDAC1/2-specific inhibitor is a compound of Formula II:

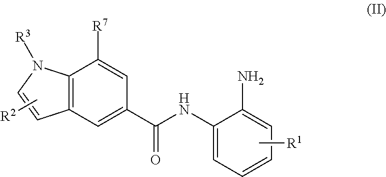

or a pharmaceutically acceptable salt thereof,
wherein,
R¹ is aryl or heteroaryl;
R² and R³ are each independently selected from C₃₋₆-cycloalkyl, C₁₋₆-alkyl-OR⁶, C₁₋₆-alkyl-C₃₋₆-cycloalkyl, C₁₋₆-alkyl-heterocycloalkyl, C₂₋₆-alkenyl;
R⁶ is H or C₁₋₆-alkyl; and
R⁷ is H or C₃₋₆-cycloalkyl.

Compounds of Formula II are represented by, but not limited to, Compound B, or pharmaceutically acceptable salts thereof.

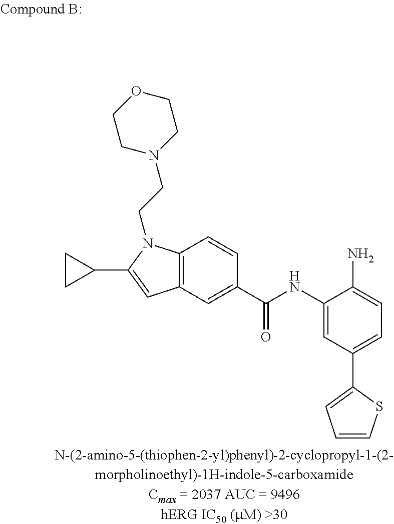 [SMILES: Nc1ccc(-c2cccs2)cc1NC(=O)c1ccc2c(c1)cc(C1CC1)n2CCN1CCOCC1]

The preparation and properties of HDAC1/2-specific inhibitors according to Formula II are provided in US Patent Publication No. 2014-0128391, the entire contents of which are incorporated herein by reference. The HDAC inhibitory profile of Compound B is found in Example 3, Table 1.

In another embodiment, the HDAC inhibitor is Compound E, or a pharmaceutically acceptable salt thereof.

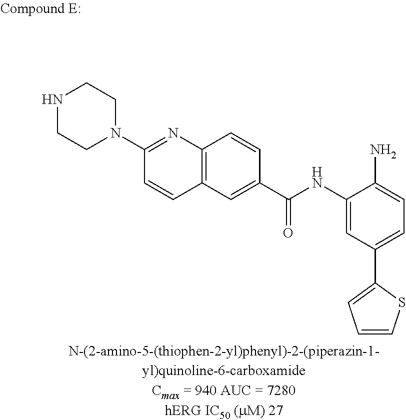 [SMILES: Nc1ccc(-c2cccs2)cc1NC(=O)c1ccc2nc(N3CCNCC3)ccc2c1]

The preparation and properties of the HDAC inhibitor Compound E are provided in US Patent Publication No. 2014-0128391, the entire contents of which are incorporated herein by reference. The HDAC inhibitory profile of Compound E is found in Example 3, Table 1.

In some embodiments, the compounds described herein are unsolvated. In other embodiments, one or more of the compounds are in solvated form. As known in the art, the solvate can be any of pharmaceutically acceptable solvent, such as water, ethanol, and the like.

### Combinations/Pharmaceutical Combinations

Provided herein are combinations for the treatment of neuroblastoma in a subject in need thereof. Provided in some embodiments are combinations, comprising an HDAC inhibitor and retinoic acid for the treatment of neuroblastoma in a subject in need thereof. In some embodiments of the combinations, the retinoic acid is ATRA, or a pharmaceutically acceptable salt thereof. In other embodiments of the combinations, the retinoic acid is 13-cis-retinoic acid, or a pharmaceutically acceptable salt thereof.

In some embodiments, the HDAC inhibitor is a compound of Formula I:

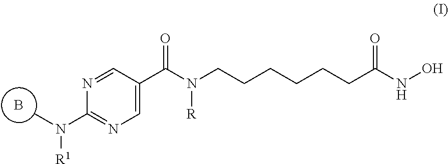

or a pharmaceutically acceptable salt thereof.

In an embodiment, the compound of Formula I is:

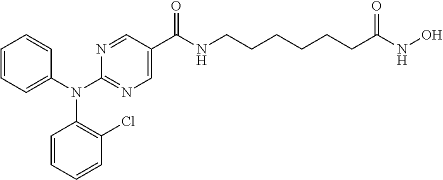 [SMILES: O=C(CCCCCCNC(=O)c1cnc(N(c2ccccc2)c2ccccc2Cl)nc1)NO]

or a pharmaceutically acceptable salt thereof.

In an embodiment, the compound of Formula I is:

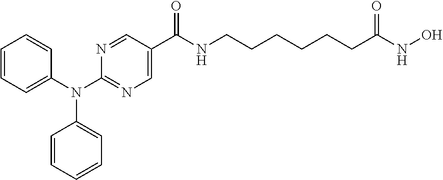 [SMILES: O=C(CCCCCCNC(=O)c1cnc(N(c2ccccc2)c2ccccc2)nc1)NO]

or a pharmaceutically acceptable salt thereof.

In other specific embodiments, the HDAC1/2-specific inhibitor is a compound of Formula II:

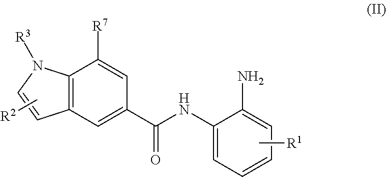

or a pharmaceutically acceptable salt thereof.

In preferred embodiments, the compound of Formula II is:

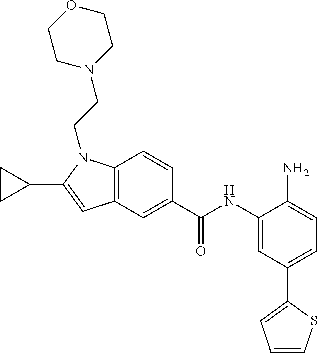 [SMILES: Nc1ccc(-c2cccs2)cc1NC(=O)c1ccc2c(c1)cc(C1CC1)n2CCN1CCOCC1]

or a pharmaceutically acceptable salt thereof.

In another preferred embodiment, the HDAC inhibitor is:

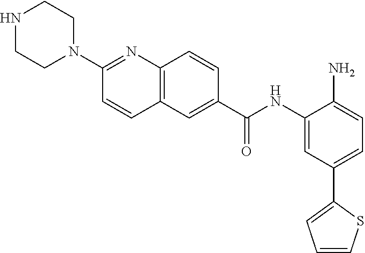 [SMILES: Nc1ccc(-c2cccs2)cc1NC(=O)c1ccc2nc(N3CCNCC3)ccc2c1]

or a pharmaceutically acceptable salt thereof.

In certain embodiment, the retinoic acid is all-trans-retinoic acid, which is also known as tretinoin and has the following structure:

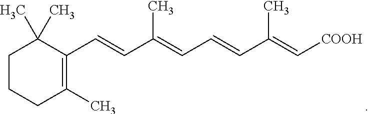 [SMILES: CC1=C(/C=C/C(C)=C/C=C/C(C)=C/C(=O)O)C(C)(C)CCC1]

In another embodiment, the retinoic acid is 13-cis-retinoic acid, or isotretinoin, having the following structure:

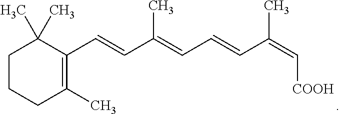 [SMILES: CC1=C(/C=C/C(C)=C/C=C/C(C)=C\C(=O)O)C(C)(C)CCC1]

In yet another embodiment, the retinoic acid is 9-cis-retinoic acid, or alitretinoin, having the following structure:

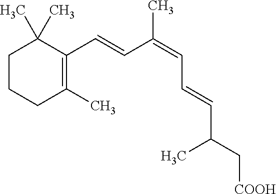 [SMILES: CC1=C(/C=C/C(C)=C\C=C\C(C)CC(=O)O)C(C)(C)CCC1]

In some embodiments of the combinations, retinoic acid can be the free acid or a pharmaceutically acceptable salt thereof.

Although the compounds of Formulas I and II, and Compound E, are depicted in their neutral forms, in some embodiments, these compounds are used in a pharmaceutically acceptable salt form. As used herein, “pharmaceutically acceptable salts” refers to derivatives of the disclosed compounds wherein the parent compound is modified by converting an existing acid or base moiety to its salt form. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Administration/Dose

In some embodiments, the HDAC inhibitor (a compound of Formula I or II, or Compound E) is administered alone. In some embodiments, the HDAC inhibitor (a compound of Formula I or II, or Compound E) is administered simultaneously with retinoic acid. Simultaneous administration typically means that both compounds enter the patient at precisely the same time. However, simultaneous administration also includes the possibility that the HDAC inhibitor and retinoic acid enter the patient at different times, but the difference in time is sufficiently miniscule that the first administered compound is not provided the time to take effect on the patient before entry of the second administered compound. Such delayed times typically correspond to less than 1 minute, and more typically, less than 30 seconds. In one example, wherein the compounds are in solution, simultaneous administration can be achieved by administering a solution containing the combination of compounds. In another example, simultaneous administration of separate solutions, one of which contains the HDAC inhibitor and the other of which contains retinoic acid, can be employed. In one example wherein the compounds are in solid form, simultaneous administration can be achieved by administering a composition containing the combination of compounds. Alternatively, simultaneous administration can be achieved by administering two separate compositions, one comprising the HDAC inhibitor and the other comprising retinoic acid.

In other embodiments, the HDAC inhibitor and retinoic acid are not administered simultaneously. In some embodiments, the HDAC inhibitor is administered before retinoic acid. In other embodiments, retinoic acid is administered before the HDAC inhibitor. In other embodiments, the first administered compound is provided time to take effect on the patient before the second administered compound is administered. Generally, the difference in time does not extend beyond the time for the first administered compound to complete its effect in the patient, or beyond the time the first administered compound is completely or substantially eliminated or deactivated in the patient.

In some embodiments, one or both of the HDAC inhibitor and retinoic acid are administered in a therapeutically effective amount or dosage. A “therapeutically effective amount” is an amount of HDAC inhibitor (a compound of Formula I or II, or Compound E) or retinoic acid that, when administered to a patient by itself, effectively treats neuroblastoma. An amount that proves to be a “therapeutically effective amount” in a given instance, for a particular subject, may not be effective for 100% of subjects similarly treated for the disease or condition under consideration, even though such dosage is deemed a “therapeutically effective amount” by skilled practitioners. The amount of the compound that corresponds to a therapeutically effective amount is strongly dependent on the type of cancer, stage of the cancer, the age of the patient being treated, and other facts. In general, therapeutically effective amounts, e.g., retinoic acid, are known in the art.

In other embodiments, one or both of the HDAC inhibitor and retinoic acid are administered in a sub-therapeutically effective amount or dosage. A sub-therapeutically effective amount is an amount of HDAC inhibitor (a compound of Formula I or II, or Compound E) or retinoic acid that, when administered to a patient by itself, does not completely inhibit over time the biological activity of the intended target.

Whether administered in therapeutic or sub-therapeutic amounts, the combination of the HDAC inhibitor and retinoic acid should be effective in treating a neuroblastoma. For example, a sub-therapeutic amount of a compound of retinoic acid can be an effective amount if, when combined with an HDAC inhibitor (a compound of Formula I or II, or Compound E), the combination is effective in the treatment of neuroblastoma. For example, a sub-therapeutic amount of a compound of retinoic acid can be an effective amount if, when combined with an HDAC inhibitor (a compound of Formula I or II, or Compound E), the combination is effective in the treatment of neuroblastoma, wherein the combination is administered at dosages that would not be effective when one or both of the compounds are administered alone, but which amounts are effective in combination.

In some embodiments, the combination of compounds exhibits a synergistic effect (i.e., greater than additive effect) in the treatment of neuroblastoma. The term “synergistic effect” refers to the action of two agents, such as, for example, an HDAC inhibitor and retinoic acid, producing an effect, for example, slowing the symptomatic progression of neuroblastoma or symptoms thereof, which is greater than the simple addition of the effects of each drug administered alone. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In preferred embodiments, the combinations and methods provided herein include an HDAC inhibitor of Formula I and retinoic acid. Thus, in one embodiment, the combinations and methods include Compound A and retinoic acid. In another embodiment, the combination and methods include Compound F and retinoic acid. In other preferred embodiments, the combinations and methods provided herein include an HDAC inhibitor of Formula II and retinoic acid. Thus, in one embodiment, the combinations and methods include Compound B and retinoic acid. In still other preferred embodiments, the combinations and methods provided herein include Compound E and retinoic acid.

In different embodiments, depending on the combination and the effective amounts used, the combination of compounds can inhibit neuroblastoma growth, achieve neuroblastoma stasis, or even achieve substantial or complete neuroblastoma regression.

While the amounts of an HDAC inhibitor and retinoic acid should result in the effective treatment of neuroblastoma, the amounts, when combined, are preferably not excessively toxic to the patient (i.e., the amounts are preferably within toxicity limits as established by medical guidelines). In some embodiments, either to prevent excessive toxicity or provide a more efficacious treatment, or both, of neuroblastoma, a limitation on the total administered dosage is provided. Typically, the amounts considered herein are per day; however, half-day and two-day or three-day cycles also are considered herein.

Different dosage regimens can be used to treat neuroblastoma. In some embodiments, a daily dosage, such as any of the exemplary dosages described above, is administered once, twice, three times, or four times a day for three, four, five, six, seven, eight, nine, or ten days. Depending on the stage and severity of the cancer, a shorter treatment time (e.g., up to five days) can be employed along with a high dosage, or a longer treatment time (e.g., ten or more days, or weeks, or a month, or longer) can be employed along with a low dosage. In some embodiments, a once- or twice-daily dosage is administered every other day. In some embodiments, each dosage contains both an HDAC inhibitor and retinoic acid to be delivered as a single dosage, while in other embodiments each dosage contains an HDAC inhibitor or retinoic acid to be delivered as separate dosages.

Compounds of Formula I or II, or Compound E, or their pharmaceutically acceptable salts or solvate forms, in pure form or in an appropriate pharmaceutical composition, can be administered via any of the accepted modes of administration or agents known in the art. The compounds can be administered, for example, orally, nasally, parenterally (intravenous, intramuscular, or subcutaneous), topically, transdermally, intravaginally, intravesically, intracistemally, or rectally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, preferably in unit dosage forms suitable for simple administration of precise dosages. A particular route of administration is oral, particularly one in which a convenient daily dosage regimen can be adjusted according to the degree of severity of the disease to be treated.

As discussed above, the HDAC inhibitor and retinoic acid pharmaceutical combination can be administered in a single unit dose or separate dosage forms. Accordingly, the phrase “pharmaceutical combination” includes a combination of two drugs in either a single dosage form or separate dosage forms, i.e., the pharmaceutically acceptable carriers and excipients described throughout the application can be combined with an HDAC inhibitor and retinoic acid in a single unit dose, as well as individually combined with an HDAC inhibitor and retinoic acid when these compounds are administered separately.

Auxiliary and adjuvant agents can include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, can also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

Solid dosage forms can be prepared with coatings and shells, such as enteric coatings and others well-known in the art. They can contain pacifying agents and can be of such composition that they release the active compound or compounds in a certain part of the intestinal tract in a delayed manner Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the HDAC inhibitors or retinoic acid described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Generally, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compound described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed. (Mack Publishing Company, Easton, Pa., 1990).

### Methods of the Invention

Provided herein are methods for treating neuroblastoma in a subject in need thereof comprising administering to the subject an HDAC inhibitor. Also provided herein are methods for treating neuroblastoma in a subject in need thereof comprising administering to the subject a pharmaceutical combination of the invention. Thus, provided herein are methods for treating neuroblastoma in a subject in need thereof comprising administering to the subject a therapeutically effective amount of an HDAC inhibitor. Also provided herein are methods for treating neuroblastoma in a subject in need thereof comprising administering to the subject a therapeutically effective amount of a combination comprising an HDAC inhibitor and retinoic acid. In one embodiment, the subject was previously refractory to retinoic acid (e.g., ATRA or 13-cis-retinoic acid).

In an embodiment, the HDAC inhibitor and retinoic acid are administered at dosages and/or over time intervals producing a synergistic effect.

The subject considered herein is typically a human. However, the subject can be any mammal for which treatment is desired. Thus, the methods described herein can be applied to both human and veterinary applications.

The terms “treating” or “treatment” indicates that the method has, at the least, mitigated abnormal cellular proliferation. For example, the method can reduce the rate of neuroblastoma growth in a patient, or prevent the continued growth or spread of the neuroblastoma, or even reduce the overall reach of neuroblastoma.

As such, in one embodiment, provided herein is a method for treating neuroblastoma in a subject in need thereof comprising administering to the subject a therapeutically effective amount of Compound A and retinoic acid, or pharmaceutically acceptable salts thereof.

In another embodiment, provided herein is a method for treating neuroblastoma in a subject in need thereof comprising administering to the subject a therapeutically effective amount of Compound F and retinoic acid, or pharmaceutically acceptable salts thereof.

In another embodiment, provided herein is a method for treating neuroblastoma in a subject in need thereof comprising administering to the subject a therapeutically effective amount of Compound B and retinoic acid, or pharmaceutically acceptable salts thereof.

In another embodiment, provided herein is a method for treating neuroblastoma in a subject in need thereof comprising administering to the subject a therapeutically effective amount of Compound E and retinoic acid, or pharmaceutically acceptable salts thereof.

In another embodiment, provided herein is a method for treating neuroblastoma in a subject in need thereof comprising administering to the subject a therapeutically effective amount of Formula I and retinoic acid, or pharmaceutically acceptable salts thereof.

In another embodiment, provided herein is a method for treating neuroblastoma in a subject in need thereof comprising administering to the subject a therapeutically effective amount of Formula II and retinoic acid, or pharmaceutically acceptable salts thereof.

In yet another embodiment, provided herein is a method for treating neuroblastoma in a subject in need thereof comprising administering to the subject a therapeutically effective amount of Formula I, or a pharmaceutically acceptable salt thereof.

In yet another embodiment, provided herein is a method for treating neuroblastoma in a subject in need thereof comprising administering to the subject a therapeutically effective amount of Formula II, or a pharmaceutically acceptable salt thereof.

In yet another embodiment, provided herein is a method for treating neuroblastoma in a subject in need thereof comprising administering to the subject a therapeutically effective amount of Compound A, or a pharmaceutically acceptable salt thereof.

In still another embodiment, provided herein is a method for treating neuroblastoma in a subject in need thereof comprising administering to the subject a therapeutically effective amount of Compound B, or a pharmaceutically acceptable salt thereof.

In another embodiment, provided herein is a method for treating neuroblastoma in a subject in need thereof comprising administering to the subject a therapeutically effective amount of Compound E, or a pharmaceutically acceptable salt thereof.

Also provided herein are methods for inhibiting migration or invasion, or both, of neuroblastoma cells. In particular, provided herein are methods for inhibiting migration or invasion, or both, of neuroblastoma cells, in a subject in need thereof. Specifically, provided herein are methods for inhibiting migration or invasion, or both, of neuroblastoma cells in a subject in need thereof comprising administering to the subject a therapeutically effective amount of an HDAC inhibitor of Formula I or II, or Compound E. Also provided herein are methods for inhibiting migration or invasion, or both, of neuroblastoma cells in a subject in need thereof comprising administering to the subject a therapeutically effective amount of a combination comprising an HDAC inhibitor of Formula I or II, or Compound E, and retinoic acid.

Provided herein are methods for decreasing cell viability of cancer cells by administering a combination comprising an HDAC inhibitor and retinoic acid.

Provided herein are methods for inducing differentiation of cancer cells by administering a combination comprising an HDAC inhibitor and retinoic acid.

Provided herein are methods for inducing apoptosis of cancer cells by administering a combination comprising an HDAC inhibitor and retinoic acid.

Provided herein are methods for decreasing cell cycle progression comprising administering a combination comprising an HDAC inhibitor and retinoic acid.

Provided herein are methods for increasing cellular apoptosis comprising administering a combination comprising an HDAC inhibitor and retinoic acid.

Provided herein are methods for suppressing transcriptional regulators in cancer comprising administering a combination therapy comprising an HDAC inhibitor and retinoic acid.

Provided herein are methods for enhancing neuroblastoma differentiation comprising administering a combination therapy comprising an HDAC inhibitor and retinoic acid. In one embodiment, differentiation is induced by a non-cytotoxic amount of the HDAC inhibitor.

Provided herein are methods for suppressing neuroblastoma proliferation comprising administering a combination therapy comprising an HDAC inhibitor and retinoic acid.

Provided herein are methods for enhancing alteration of the binding position of retinoic acid receptor to chromatin comprising administering a combination comprising an HDAC inhibitor and retinoic acid.

Provided herein are methods for reducing Wnt signaling comprising administering a combination comprising an HDAC inhibitor and retinoic acid.

Provided herein are methods for enhancing one or more of a signaling pathway selected from the group consisting of KRAS signaling (up), protein secretion, pancreas beta cells, TNFA signaling via NFKB, bile acid metabolism, complement, coagulation, adipogenesis, estrogen response (early), fatty acid metabolism, estrogen response (late), epithelial mesenchymal transition, IL2-STATS signaling, interferon gamma response, apoptosis, interferon alpha response, and IL6-JAK-STAT3, or suppressing one or more of a signaling pathway selected from the group consisting of MYC target (V2), E2F target, MYC target (V1), and DNA repair comprising administering a combination therapy comprising an HDAC inhibitor and retinoic acid.

Provided herein are methods for enhancing one or more of a signaling pathway selected from the group consisting of KRAS signaling (up), protein secretion, pancreas beta cells, TNFA signaling via NFKB, bile acid metabolism, complement, coagulation, adipogenesis, estrogen response (early), fatty acid metabolism, estrogen response (late), epithelial mesenchymal transition, IL2-STATS signaling, interferon gamma response, apoptosis, interferon alpha response, and IL6-JAK-STAT3, or suppressing one or more of a signaling pathway selected from the group consisting of MYC target (V2), E2F target, MYC target (V1), and DNA repair comprising administering an HDAC inhibitor.

Provided herein are methods for enhancing one or more of a signaling pathway selected from the group consisting of KRAS signaling (up), protein secretion, pancreas beta cells, TNFA signaling via NFKB, bile acid metabolism, complement, coagulation, adipogenesis, estrogen response (early), fatty acid metabolism, estrogen response (late), epithelial mesenchymal transition, IL2-STATS signaling, interferon gamma response, apoptosis, interferon alpha response, and IL6-JAK-STAT3, or suppressing one or more of a signaling pathway selected from the group consisting of MYC target (V2), E2F target, MYC target (V1), and DNA repair comprising administering retinoic acid.

Provided herein are methods for enhancing one or more of a signaling pathway selected from the group consisting of KRAS signaling (up), protein secretion, pancreas beta cells, TNFA signaling via NFKB, bile acid metabolism, complement, coagulation, adipogenesis, estrogen response (early), fatty acid metabolism, estrogen response (late), epithelial mesenchymal transition, IL2-STATS signaling, interferon gamma response, apoptosis, interferon alpha response, and IL6-JAK-STAT3, and suppressing one or more of a signaling pathway selected from the group consisting of MYC target (V2), E2F target, MYC target (V1), and DNA repair comprising administering a combination therapy comprising an HDAC inhibitor and retinoic acid.

Provided herein are methods for enhancing one or more of a signaling pathway selected from the group consisting of KRAS signaling (up), protein secretion, pancreas beta cells, TNFA signaling via NFKB, bile acid metabolism, complement, coagulation, adipogenesis, estrogen response (early), fatty acid metabolism, estrogen response (late), epithelial mesenchymal transition, IL2-STATS signaling, interferon gamma response, apoptosis, interferon alpha response, and IL6-JAK-STAT3, and suppressing one or more of a signaling pathway selected from the group consisting of MYC target (V2), E2F target, MYC target (V1), and DNA repair comprising administering an HDAC inhibitor.

Provided herein are methods for enhancing one or more of a signaling pathway selected from the group consisting of KRAS signaling (up), protein secretion, pancreas beta cells, TNFA signaling via NFKB, bile acid metabolism, complement, coagulation, adipogenesis, estrogen response (early), fatty acid metabolism, estrogen response (late), epithelial mesenchymal transition, IL2-STATS signaling, interferon gamma response, apoptosis, interferon alpha response, and IL6-JAK-STAT3, and suppressing one or more of a signaling pathway selected from the group consisting of MYC target (V2), E2F target, MYC target (V1), and DNA repair comprising administering retinoic acid.

### Kits

In other embodiments, kits are provided. Kits according to the invention include package(s) comprising compounds or compositions of the invention. In some embodiments, kits comprise an HDAC inhibitor, or a pharmaceutically acceptable salt thereof, and retinoic acid, or a pharmaceutically acceptable salt thereof.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering compounds or compositions of the invention to a patient. Kits also can comprise instructions for approved uses of compounds herein by regulatory agencies, such as the United States Food and Drug Administration. Kits can also contain labeling or product inserts for the compounds. The package(s) or any product insert(s), or both, can themselves be approved by regulatory agencies. The kits can include compounds in the solid phase or in a liquid phase (such as buffers provided) in a package. The kits can also include buffers for preparing solutions for conducting the methods, and pipettes for transferring liquids from one container to another.

### EXAMPLES

Examples have been set forth below for the purpose of illustration and to describe certain specific embodiments of the invention. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations or methods of the invention can be made without departing from the spirit of the invention and the scope of the appended claims. Definitions of the variables in the structures in the schemes herein are commensurate with those of corresponding positions in the formulae presented herein.

It is demonstrated herein that next generation selective and orally bioavailable HDAC1/2 inhibitors can induce gene expression changes in neuroblastoma cells consistent with differentiation. The action of HDAC1/2 inhibitors potently enhances the retinoic acid differentiation effect at sub-optimal concentrations of retinoic acid or HDAC inhibitor, as well as with intermittent (pulse) HDAC1/2 inhibition. Retinoic acid alone and in combination with HDAC1/2 inhibitors is able to slow cell proliferation in long term growth assays and alter morphology in a manner consistent with differentiation. A gene expression pattern associated with retinoic acid-induced neuroblastoma differentiation is similarly induced by inhibition of HDAC1/2. The observed enhancement of differentiation by selective HDAC1/2 inhibitors occurs at concentrations below that required for cell death as evidenced by viability assays and caspase 3/7 activation. Acute toxicity is induced by elevated concentrations of HDAC1/2 inhibitors, and synergy is observed in combination with retinoic acid. Ongoing studies exploring global gene expression changes, ChIP-seq examining retinoic acid receptor and HDAC1/2 chromatin binding, and activity of the selective HDAC1/2 inhibitor in combination with retinoic acid in animal models of neuroblastoma is discussed. Taken together, these findings support a role for selective HDAC1/2 inhibitors in combination with retinoic acid for the treatment of patients with high risk neuroblastoma.

The syntheses of the compounds of Formula I (Compound A) are provided in PCT/US2011/021982; this application is incorporated herein by reference in its entirety. The syntheses of compounds of Formula II (Compound B) are provided in U.S. Patent Publication No. 2014-0128391; this application is incorporated herein by reference in its entirety.

### Example 1

### Synthesis of 2-((2-chlorophenyl)(phenyl)amino)-N-(7-(hydroxyamino)-7-oxoheptyl)pyrimidine-5-carboxamide (Compound A)

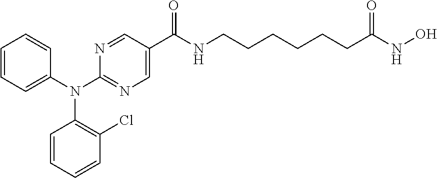 [SMILES: O=C(CCCCCCNC(=O)c1cnc(N(c2ccccc2)c2ccccc2Cl)nc1)NO]

### Reaction Scheme:

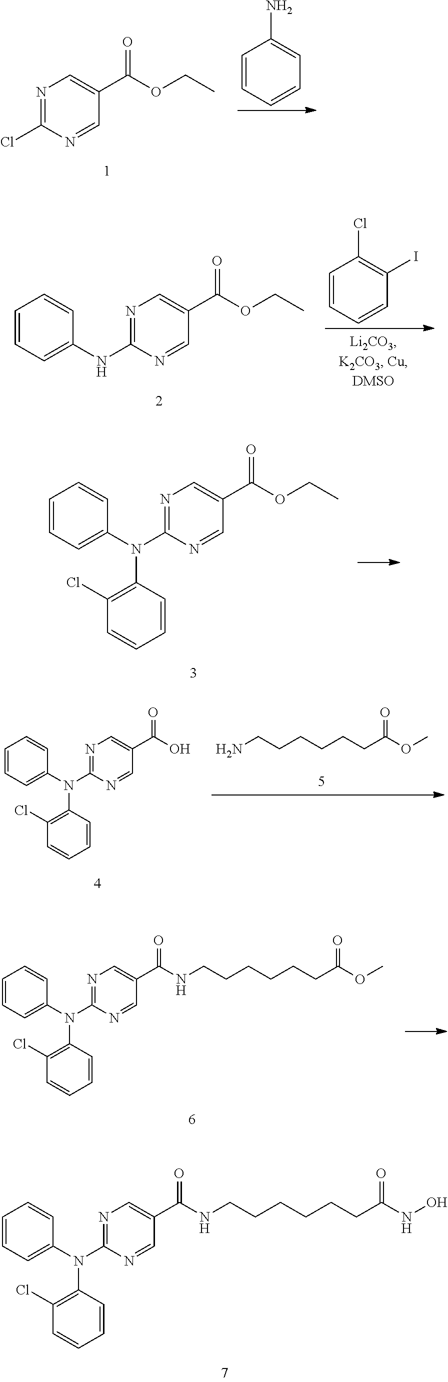

### Experimental Procedure

Synthesis of Intermediate 2: A mixture of aniline (3.7 g, 40 mmol), compound 1 (7.5 g, 40 mmol), and K₂CO₃ (11 g, 80 mmol) in DMF (100 ml) was degassed and stirred at 120° C. under N₂ overnight. The reaction mixture was cooled to r.t. and diluted with EtOAc (200 ml), then washed with saturated brine (200 ml×3). The organic layers were separated and dried over Na₂SO₄, evaporated to dryness and purified by silica gel chromatography (petroleum ethers/EtOAc=10/1) to give the desired product as a white solid (6.2 g, 64%).

Synthesis of Intermediate 3: A mixture of compound 2 (69.2 g, 1 equiv.), 1-chloro-2-iodobenzene (135.7 g, 2 equiv.), Li₂CO₃ (42.04 g, 2 equiv.), K₂CO₃ (39.32 g, 1 equiv.), Cu (1 equiv. 45 μm) in DMSO (690 ml) was degassed and purged with nitrogen. The resulting mixture was stirred at 140° C. Work-up of the reaction gave compound 3 at 93% yield.

Synthesis of Intermediate 4: 2N NaOH (200 ml) was added to a solution of compound 3 (3.0 g, 9.4 mmol) in EtOH (200 ml). The mixture was stirred at 60° C. for 30 min After evaporation of the solvent, the solution was neutralized with 2N HCl to give a white precipitate. The suspension was extracted with EtOAc (2×200 ml), and the organic layers were separated, washed with water (2×100 ml), brine (2×100 ml), and dried over Na₂SO₄. Removal of the solvent gave a brown solid (2.5 g, 92%).

Synthesis of Intermediate 6: A mixture of compound 4 (2.5 g, 8.58 mmol), compound 5 (2.52 g, 12.87 mmol), HATU (3.91 g, 10.30 mmol), and DIPEA (4.43 g, 34.32 mmol) was stirred at r.t. overnight. After the reaction mixture was filtered, the filtrate was evaporated to dryness and the residue was purified by silica gel chromatography (petroleum ethers/EtOAc=2/1) to give a brown solid (2 g, 54%).

Synthesis of 2-((2-chlorophenyl)(phenyl)amino)-N-(7-(hydroxyamino)-7-oxoheptyl)pyrimidine-5-carboxamide (Compound A): A mixture of the compound 6 (2.0 g, 4.6 mmol), sodium hydroxide (2N, 20 mL) in MeOH (50 ml) and DCM (25 ml) was stirred at 0° C. for 10 min Hydroxylamine (50%) (10 ml) was cooled to 0° C. and added to the mixture. The resulting mixture was stirred at r.t. for 20 min After removal of the solvent, the mixture was neutralized with 1M HCl to give a white precipitate. The crude product was filtered and purified by pre-HPLC to give a white solid (950 mg, 48%).

### Example 2

### Synthesis of N-(2-amino-5-(thiophen-2-yl)phenyl)-2-cyclopropyl-1-(2-morpholinoethyl)-1H-indole-5-carboxamide (Compound B)

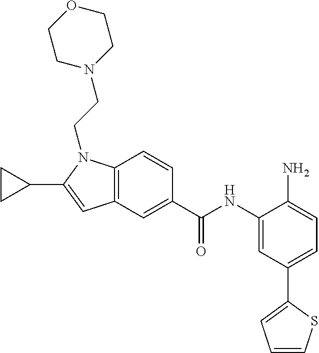 [SMILES: Nc1ccc(-c2cccs2)cc1NC(=O)c1ccc2c(c1)cc(C1CC1)n2CCN1CCOCC1]

### Reaction Scheme

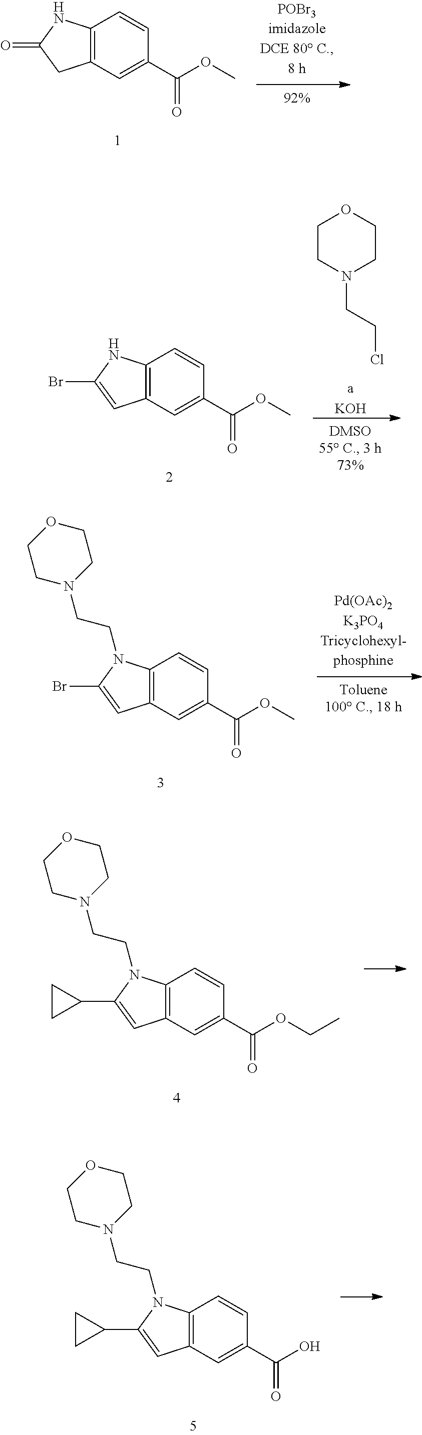

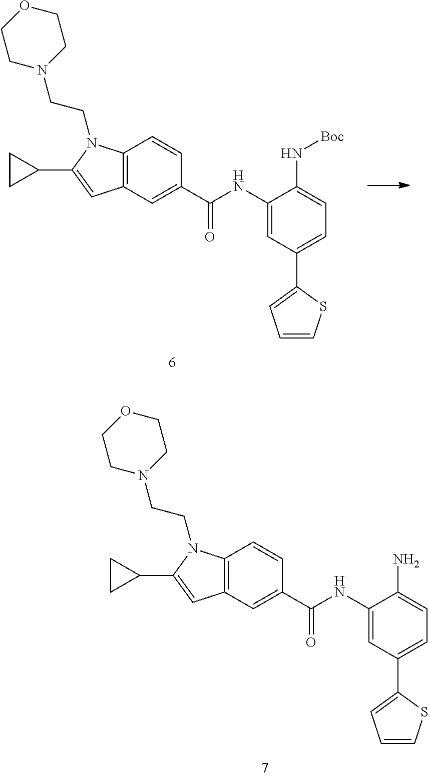 [SMILES: CC(C)(C)OC(=O)Nc1ccc(-c2cccs2)cc1NC(=O)c1ccc2c(c1)cc(C1CC1)n2CCN1CCOCC1 ; Nc1ccc(-c2cccs2)cc1NC(=O)c1ccc2c(c1)cc(C1CC1)n2CCN1CCOCC1]

### Experimental Procedure

Step 1: To a solution of compound 1 in DCE was added POBr₃ and imidazole. The reaction was stirred at 80° C. overnight. Water and DCM were added to the reaction, and the organic layer was separated, washed with brine, and dried under reduced pressure to give compound 2.

Step 2: To a solution of compound 2 in DMSO was added compound a and KOH. The resulting reaction mixture was stirred at 45° C. for 4 h, quenched with H₂O, and extracted with EA. The combined organic layers were purified by gel chromatography to yield the desired product, compound 3.

Step 3: A mixture of compound 3, cyclopropyl boronic acid, Pd(OAc)₂, tricyclohexylphosphine, and K₃PO₄ in toluene and water was stirred at 100° C. under N₂ atmosphere overnight. The mixture was cooled, filtered, and concentrated to obtain a residue, which was purified by Prep-TLC to get compound 4.

Step 4: A mixture of compound 4 and NaOH in EtOH and THF was stirred at 60° C. for 5 h. The mixture was concentrated to obtain a residue, to which was added aq. sat. citric acid and extracted with EA. The organic layers were separated, dried, filtered and concentrated to obtain compound 5.

Step 5: A mixture of compound 5, tert-butyl 2-amino-4-(thiophen-2-yl)phenylcarbamate, HOAT, EDCI, and DIPEA in DMF was stirred at 55° C. for overnight. Water was added to the mixture, and extracted with EA. The organic layers were separated, dried, filtered, and concentrated to get a residue, which was purified by Prep-TLC to afford compound 6.

Step 6: To a solution of compound 6 in DCM was added TFA and stirred at r.t. for 1 h. The mixture was concentrated to obtain a residue, which was purified by Prep-HPLC to afford compound 7. ¹H NMR (500 MHz, DMSO) δ 9.63 (s, 1H), 8.16 (s, 1H), 7.79-7.73 (m, 1H), 7.51 (d, J=2.1 Hz, 2H), 7.36 (d, J=5.1 Hz, 1H), 7.29 (dd, J=8.3, 2.1 Hz, 1H), 7.25 (d, J=3.5 Hz, 1H), 7.05 (dd, J=5.0, 3.6 Hz, 1H), 6.82 (d, J=8.3 Hz, 1H), 6.24 (s, 1H), 5.12 (s, 2H), 4.43 (s, 2H), 3.57 (s, 5H), 2.77-2.58 (m, 2H), 2.09 (s, 1H), 1.02 (d, J=8.0 Hz, 2H), 0.76 (d, J=4.4 Hz, 2H). LCMS: m/z=487.2 (M+H)+.

### Example 3

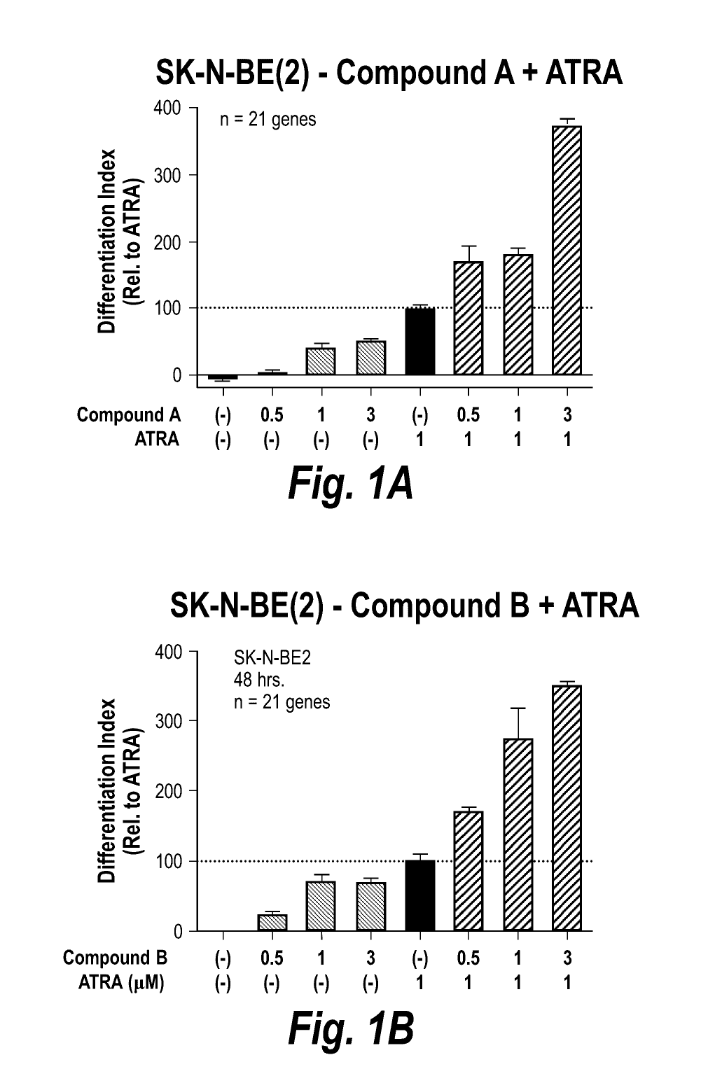

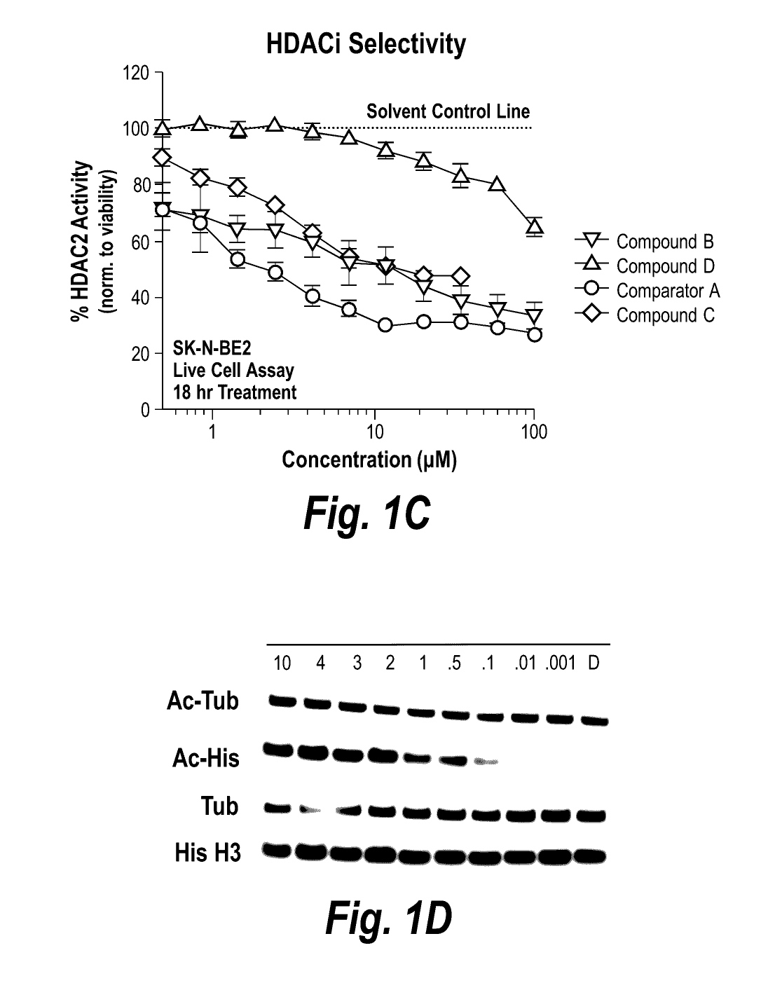

### Targeted HDAC1/2 Inhibitors Combine with ATRA to Enhance Genetic Differentiation Markers (48 Hours) (FIG. 1)

A subset of the fingerprint genes are assessed for each treatment group, and a score is assigned to each gene based on the degree of expression and weighted with a (+) or (−) sign based on if it increased or decreased as predicted. The scores are summed to generate a single Index value for each treatment group, set to ATRA signal and compared. It is observed that HDAC1/2i induce gene expression consistent with differentiation and that in combination with ATRA, the differentiation effect is enhanced.

Compound B inhibited HDAC isoforms 1 & 2 in a biochemical assay (Table 1) as well as HDAC2 activity in live cells with potency in the 0.5-3 μM range (FIG. 1C). Further, Compound B induces histone acetylation in neuroblastoma cells as studied by western blot at 24 hours (FIG. 1D). A differentiation Index Score based on a gene signature indicates that Compound B induces gene expression changes consistent with differentiation (FIG. 1B), and that the effect is enhanced in combination with retinoic acid.

TABLE 1
Biochemical Activity of Isoform-Selective
HDAC Inhibitors (IC₅₀ in nM)

| | HDAC1 | HDAC2 | HDAC3 | HDAC6 |
|---|---|---|---|---|
| Compound A | 33 | 54 | 61 | 5 |
| Compound B | 6 | 36 | 445 | — |
| Compound C | 2000 | 619 | 57 | — |
| Compound D* | 2123 | 2570 | 11223 | 7 |
| Compound E | 4 | 15 | 114 | — |
| Comparator A (MS-275) | 115 | 86 | 26 | |

*Compound D: 2-((1-(3-fluorophenyl)cyclohexyl)amino)-N-hydroxypyrimidine-5-carboxamide

Neuroblastoma cells were treated with the indicated compounds and a basket of genes associated with differenation were monitored (FIG. 1A). The Differenation Index is a metric that describes the global movement of genes associated with differenation and an increased score suggests expression patterns associated with retinoic acid induced differentation. The combination of the listed compounds with retinoic acid results in a greater differentation index score, illustrating enhanced differentiation in the combination setting.

### Example 4

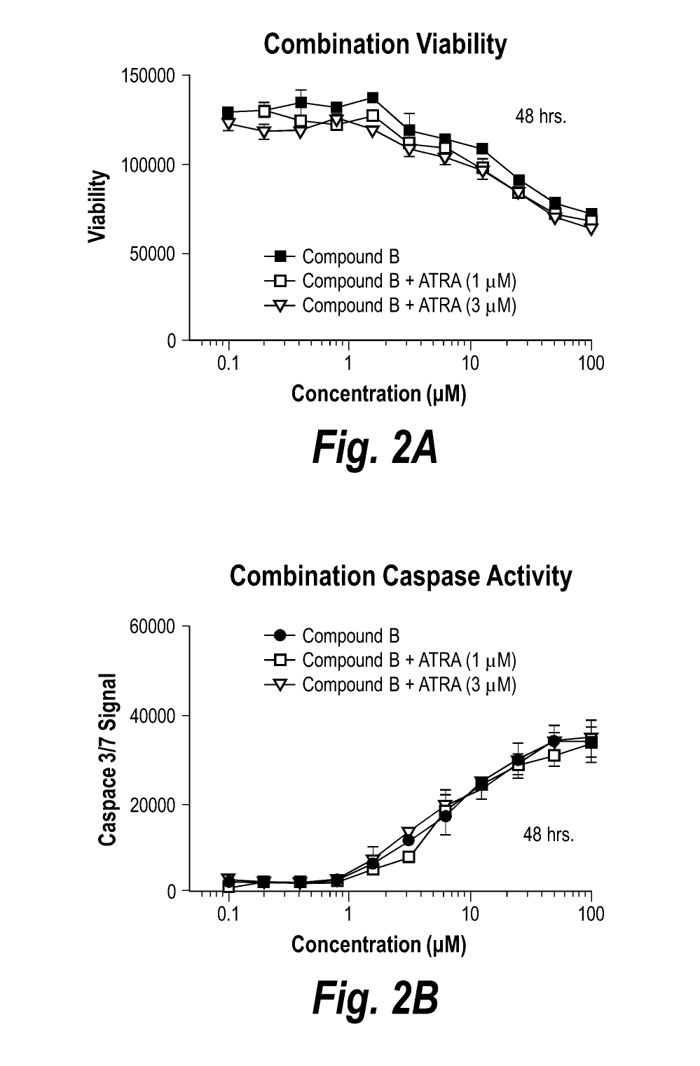

### HDAC1/2i Toxicity is Independent of Retinoic Acid and Occurs at Concentrations Greater than Those Needed for Differentiation (FIG. 2)

Compound B caused tumor cell death at concentrations ≧2 μM, which is greater than what is needed to induce differentiation (FIG. 2A). The addition of 1 μM or 3 μM of ATRA, which has potent differentiation activity, has little effect on the HDAC1/2i mediated toxicity. Similar observations were made when assessing caspase activation, with an increase in activity at concentrations ≧2 μM and little to no enhancement by retinoic acid (FIG. 2B). These results suggest that acute toxicity is driven by HDAC1/2i and is independent of retinoic acid activity.

### Example 5

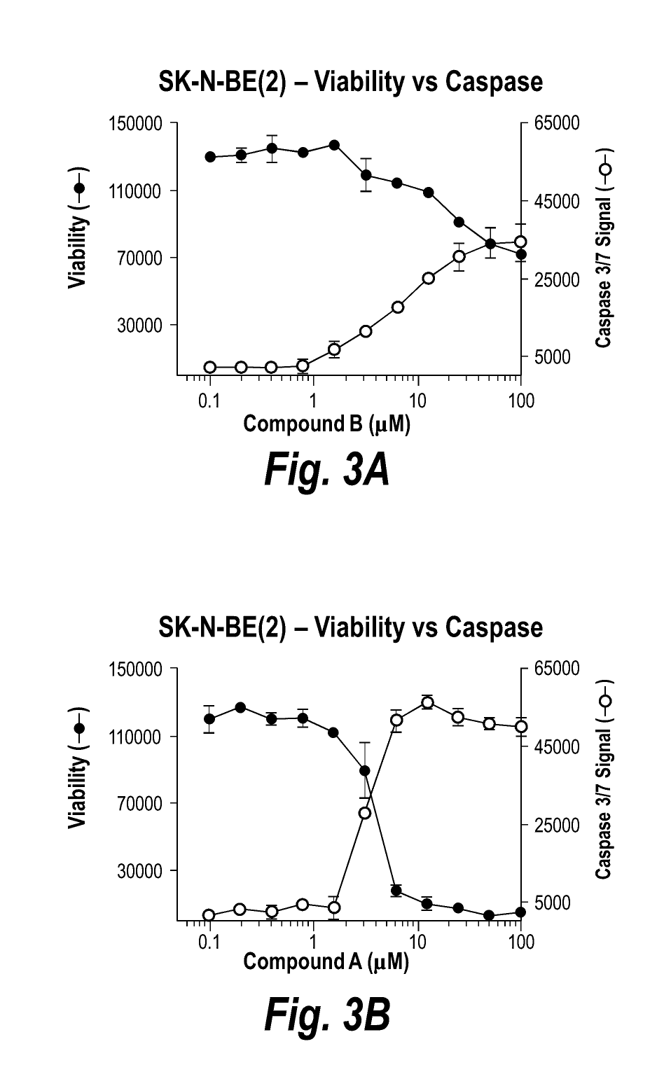

### Differentiation is Induced by Non-Cytotoxic Concentrations of Compound A and Compound B (48 Hours) (FIG. 3)

Cells were cultured for 48 hours at the indicated concentration of drug, and then cell viability was measured by Promega CellTiter Glo assay and caspase activity was measured by Promega Caspase3/7 Glo assay. Minimal cell death and caspase 3/7 induction is observed at 1 μM of Compound B or Compound A, which are concentrations that can induce differentiation (FIGS. 3A and 3B). Anti-tumor activity is evident at 3 μM of treatment.

### Example 6

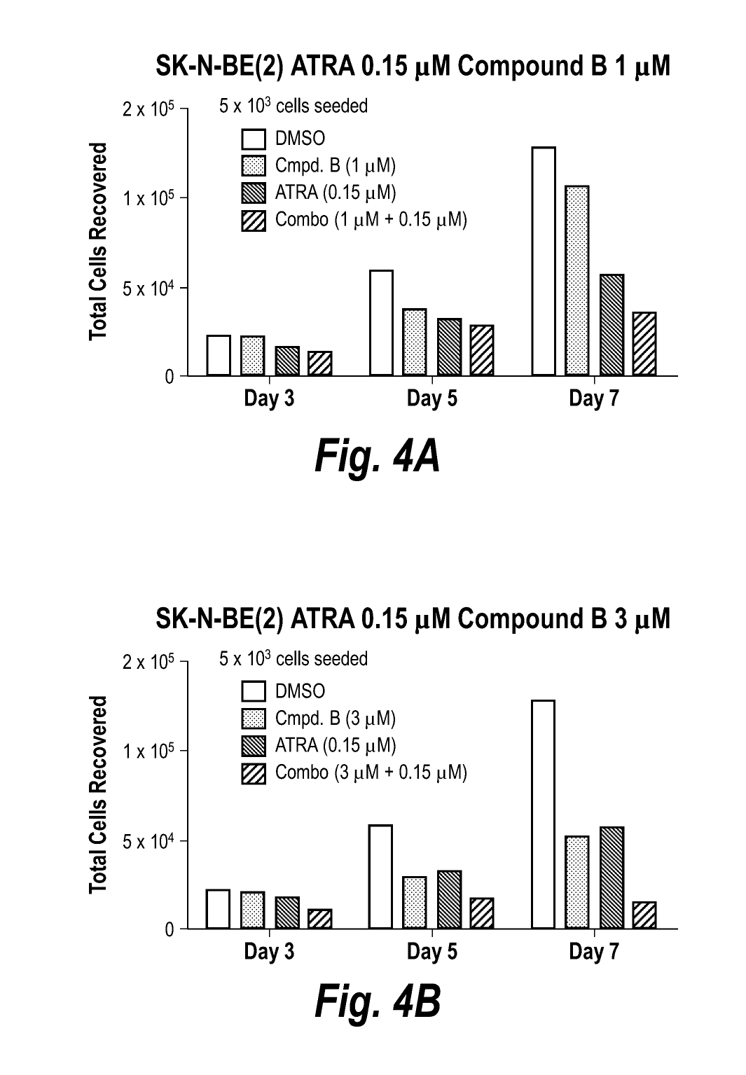

### Compound B Combines with ATRA to Enhance Suppression of Neuroblastoma Proliferation (FIG. 4)

Cells were cultured over a 7 day period and manually counted at days 3, 5, and 7. Both 1 μM and 3 μM of Compound B alone can suppress proliferation similar to ATRA, and the combination enhances suppression (FIGS. 4A and 4B). Combination effect of Compound B/ATRA on proliferation is evident at all of the time points assessed.

HDAC1/2i Enhances ATRA-mediated Suppression of Proliferation. Compound B caused a decrease in proliferation over time at concentrations that induce differentiation (FIG. 4A) Enhanced effects are observed with the Compound B/ATRA combination, particularly after extended time. The effects were enhanced by higher concentrations of HDAC1/2i (FIG. 4B).

### Example 7

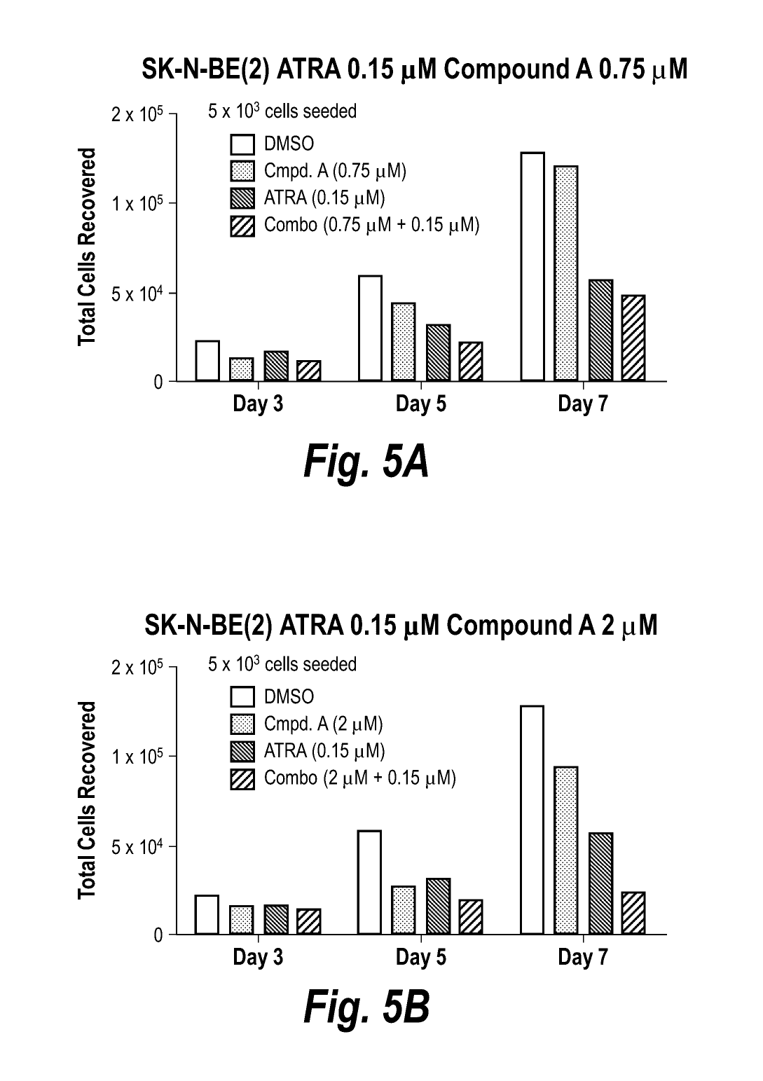

### Compound a Combines with ATRA to Enhance Suppression of Neuroblastoma Proliferation (FIG. 5)

Cells were cultured over a 7 day period and manually counted at days 3, 5, and 7. Both 0.75 μM and 2 μM of Compound A alone can suppress proliferation similar to ATRA, and the combination enhances suppression (FIGS. 5A and 5B). Combination effect of Compound A/ATRA on proliferation is evident at all of the time points assessed.

### Example 8

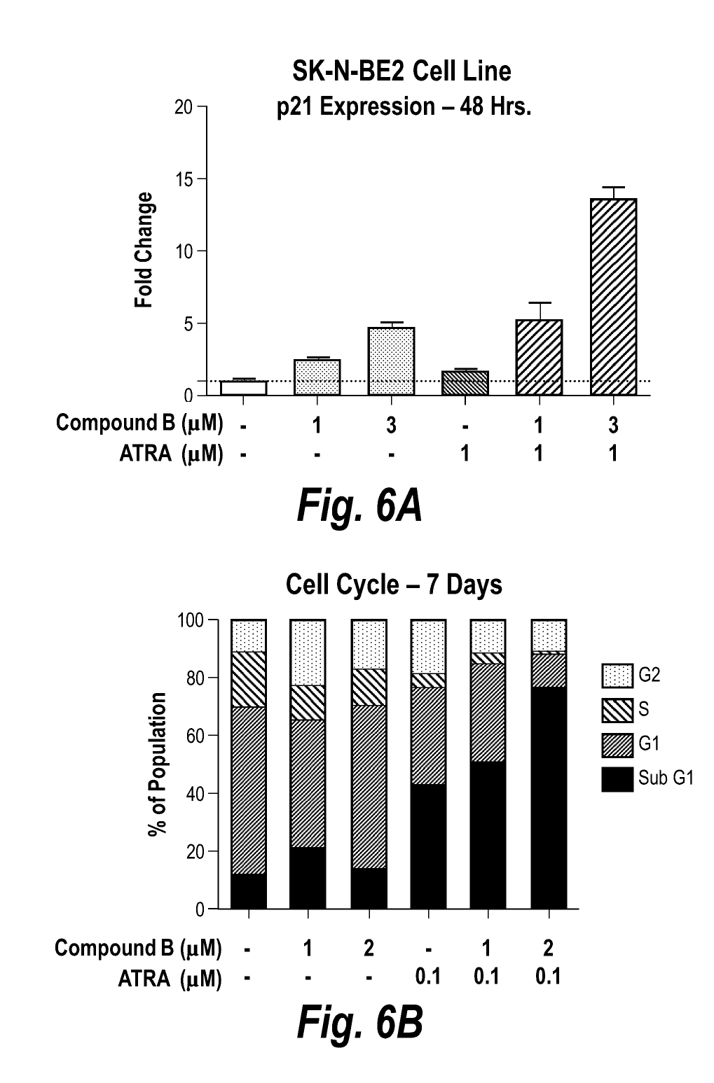

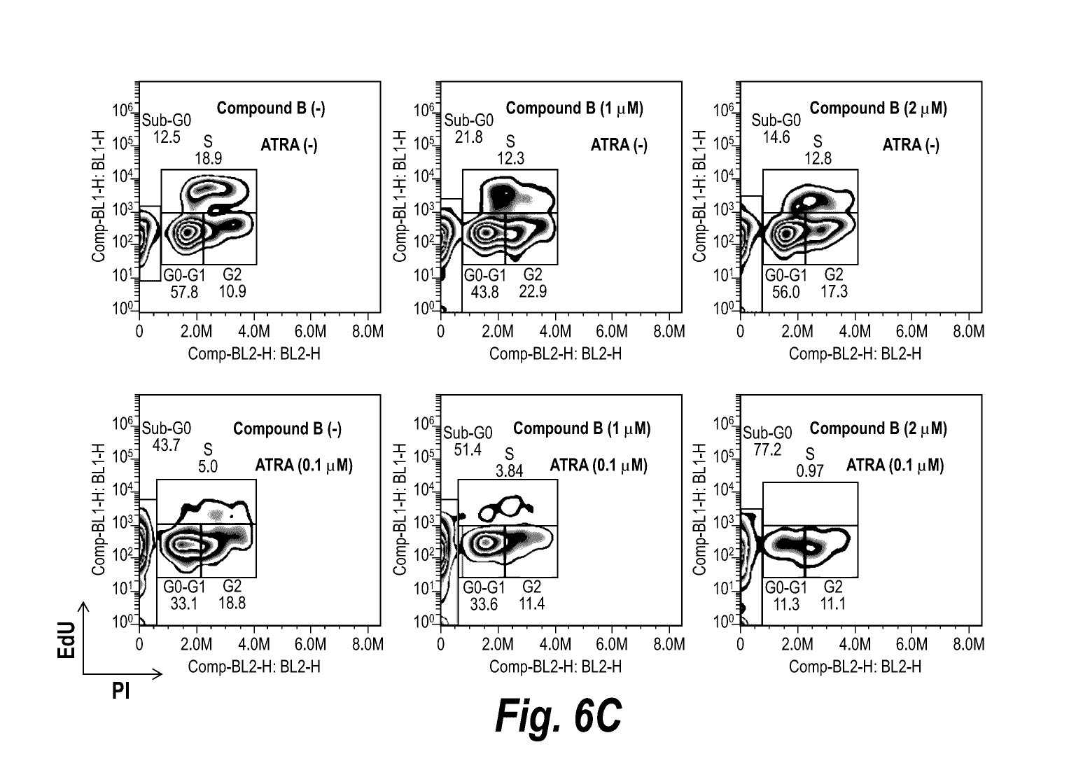

### Compound B Enhances ATRA-Mediated Suppression of Proliferation and Cell Cycle Arrest (FIG. 6)

Cells were cultured for 7 days with the indicated treatments and then stained with the Invitrogen Click-iT EdU kit and the Invitrogen FxCycle PR/Rnase kits to assess frequency of cells in each stage of the cell cycle. Single-agent ATRA reduces s-phase frequency and increases the sub-G1 population. The effect is enhanced by Compound B in a dose dependent manner.

Compound B induced increased expression of the master cell cycle regulator p21 as a single agent and in a dose-dependent manner (FIG. 6A). In combination with ATRA, the effect on p21 is enhanced at concentrations that induce gene expression changes consistent with differentiation. At 7 days of treatment, combination effects are observed on the cell cycle, with a decreasing frequency of s-phase cells and an increasing sub-G1 population (FIG. 6B). Neuroblastoma cells were treated with the indicated compounds and assessed for their cell cycle characteristics (FIG. 6C). EDU is increased in cells in s-phase while PI intensity indicates relative levels of DNA. Using these metrics, we illustrate the frequency of cells cells in the indicated states of the cell cycle. These data indicated that the combination of the indicated compounds with ATRA cause a reduction in the frequency of s-phase cells, suggesting decreased growth, while increasing the sub-G1 population, suggesting cell death.

### Example 9

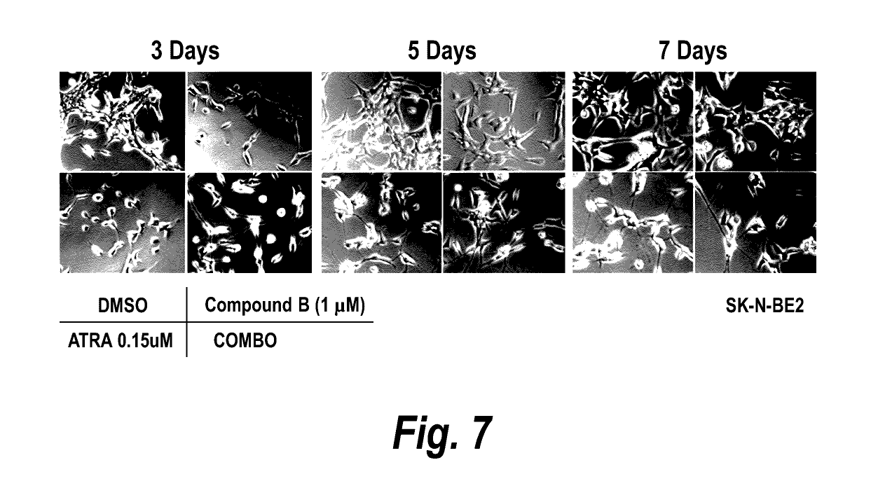

### Compound B Combines with ATRA to Enhance Morphology Changes Consistent with Differentiation (FIG. 7)

Cells were cultured for 3, 5, and 7 days with the indicated treatments. Images were captured on an inverted microscope. Dendrite outgrowth induced by ATRA is enhanced by the Compound B/ATRA combination, particularly at the 3 day time point. Retinoic acid caused the outgrowth of dendrites over time, with the strongest effects at 5 and 7 days of treatment. Compound B as a single agent does not alter morphology, but surprisingly, in combination with ATRA, Compound B enhances the ability of ATRA to induce morphology changes consistent with differentiation. The HDAC1/2i enhancement effect on retinoic acid is particularly noticeable at earlier time points.

### Example 10

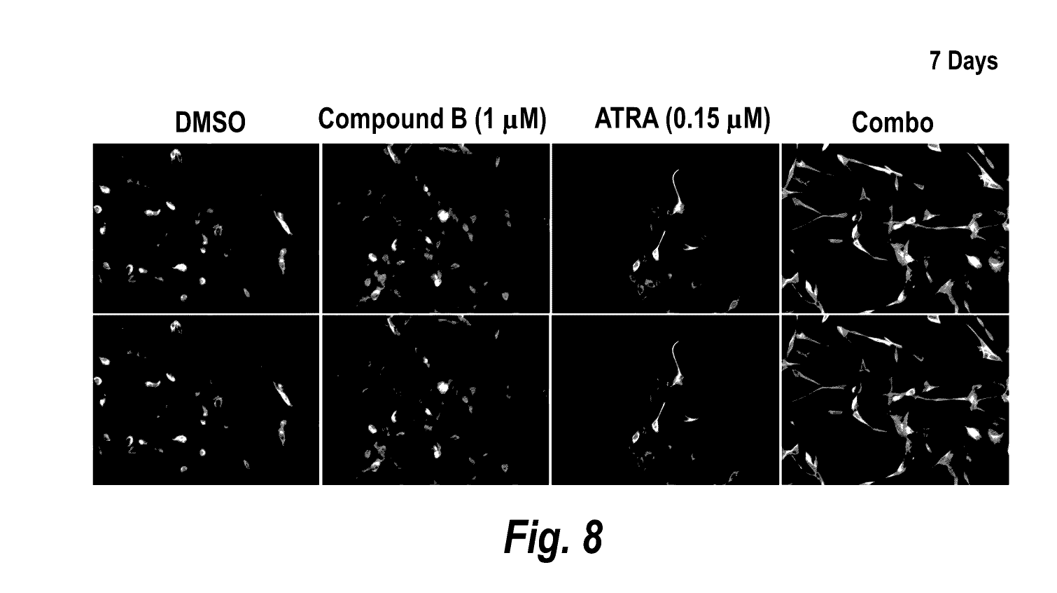

### Compound B Combines with ATRA to Enhance NF-M Staining (FIG. 8)

Cells were cultured for 7 days with the indicated treatments on glass slides. Cells were fixed and stained with antibodies for NF-M and nucleus. Images were captured on a fluorescent microscope. Compound B caused a general increase in NF-M staining. The combination of ATRA and Compound B cause both increased neurite formation and increased NF-M staining.

### Example 11

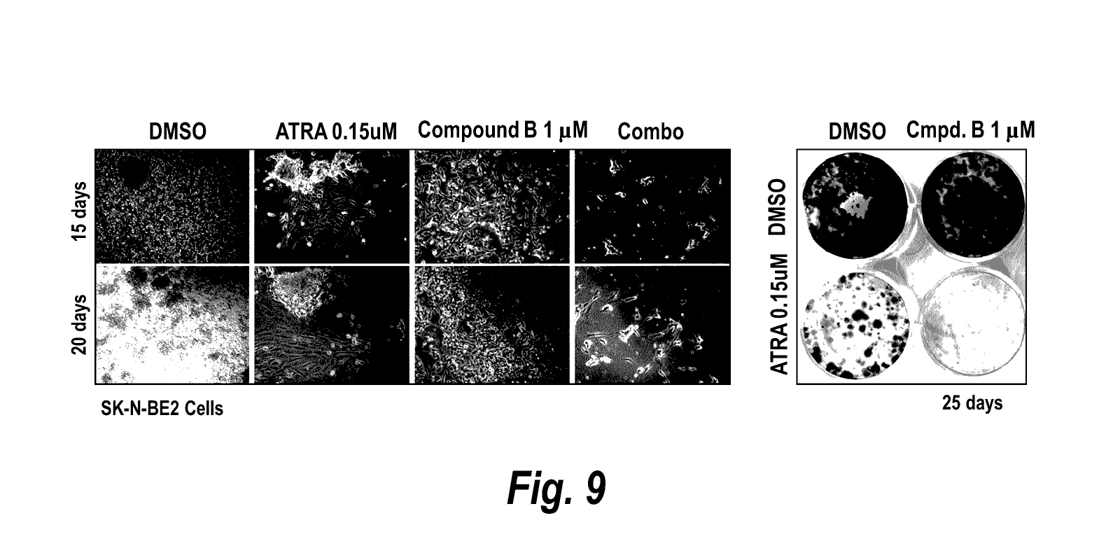

### Compound B Combines with ATRA to Reduce Neuroblastoma Outgrowth (FIG. 9)

Cells were cultured for 15, 20 or 25 days with the indicated treatments. Cells were imaged on an inverted microscope at days 15 and 20. On day 25, the cells were stained with Crystal Violet dye and imaged by cell phone camera Importantly, the combination of ATRA and Compound B suppressed the growth of ATRA-resistant colonies.

### Example 12

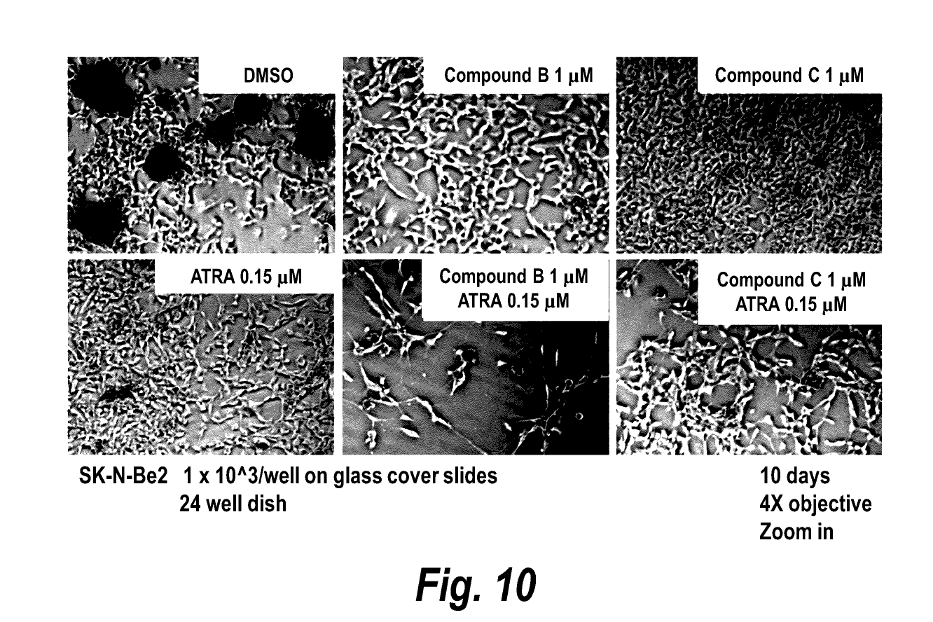

### Compound B (HDAC1/2i) Shows Stronger Combination Effect with ATRA than Compound C (HDAC3i) on Cell Proliferation and Dendrite Outgrowth (FIG. 10)

Cells were cultured for 15, 20 or 25 days with the indicated treatments. Cells were imaged on an inverted microscope at days 15 and 20. On day 25, the cells were stained with Crystal Violet dye and imaged.

### Example 13

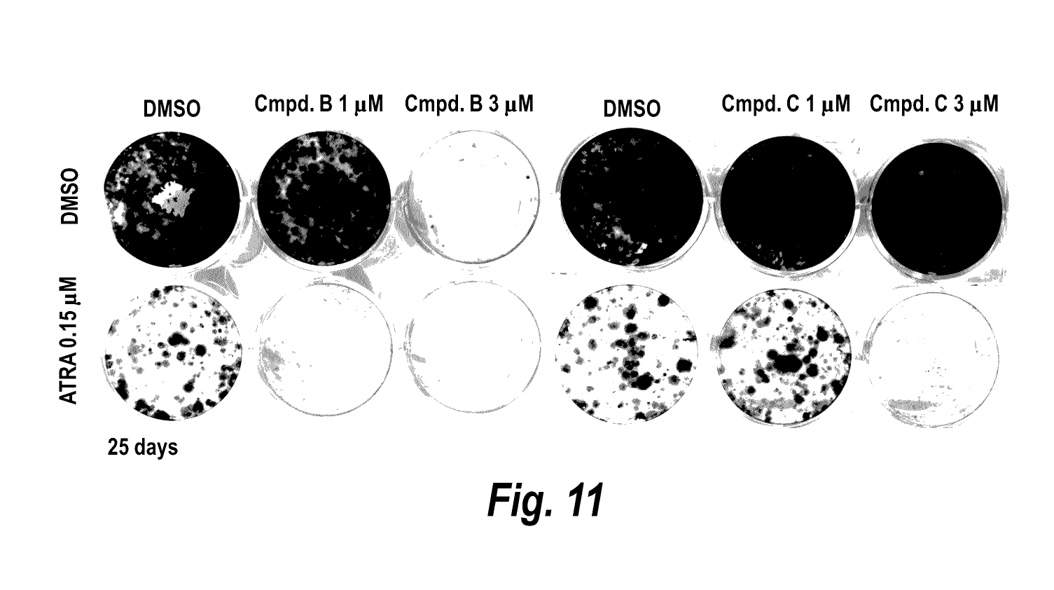

### Compound B/ATRA Combination Completely Blocks Cluster Formation of Neuroblastoma at Concentrations Lower than Compound C (FIG. 11)

Cells were cultured for 15, 20 or 25 days with the indicated treatments. Cells were imaged on an inverted microscope at days 15 and 20. On day 25, the cells were stained with Crystal Violet dye and imaged. It was observed that the combination activity is mediated by HDAC1/2i.

Neuroblastoma cells were treated with Compound B or the HDAC3-selective inhibitor Compound C in a long term growth assay. Compound B at 1 μM of exposure reduced neuroblastoma cell growth as a single agent and strongly suppressed retinoic acid resistant colonies in a combination setting (FIG. 11). Compound C, in contrast, required higher concentrations to mediate a similar effect (FIG. 11). These data suggest that at equimolar treatments, HDAC1/2i more potently enhances retinoic acid than HDAC3i.

### Example 14

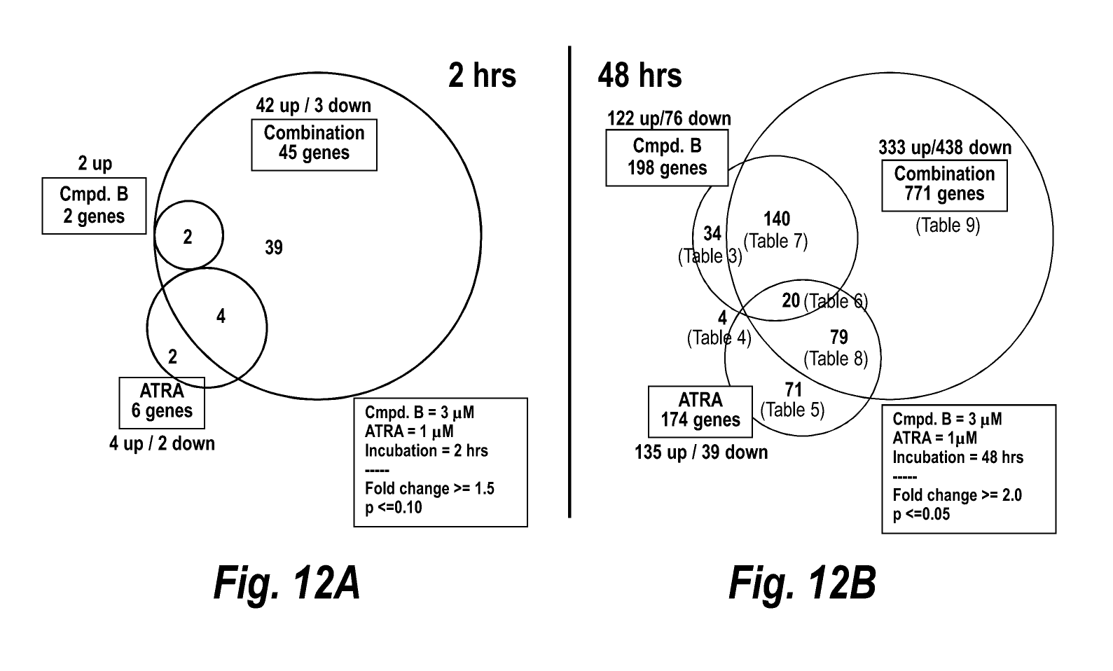

### Gene Expression Changes are Enhanced by the Combination of HDAC1/2i and ATRA Relative to Single Agent Treatment (FIG. 12)

Gene expression from cells treated with ATRA single agent and combination of ATRA and HDAC1/2i were assessed at 2 hrs and 48 hrs of treatment (FIG. 12A). The 48 hr results were assessed by gene set enrichment analysis against the Broad Institute C6 Msig database, which revealed several differentially regulated pathways involved in development, survival and differentiation (FIG. 12B). A function analysis of the data using the Ingenuity IPA platform was performed by Qiagen; overlap is observed in the top hits between treatment groups.

Genes that statistically increase over 2-fold relative to the DMSO control were mapped on a Venn diagram (FIG. 12B). Genes in each section of the Venn diagram of FIG. 12B, are listed in Tables 3-9.

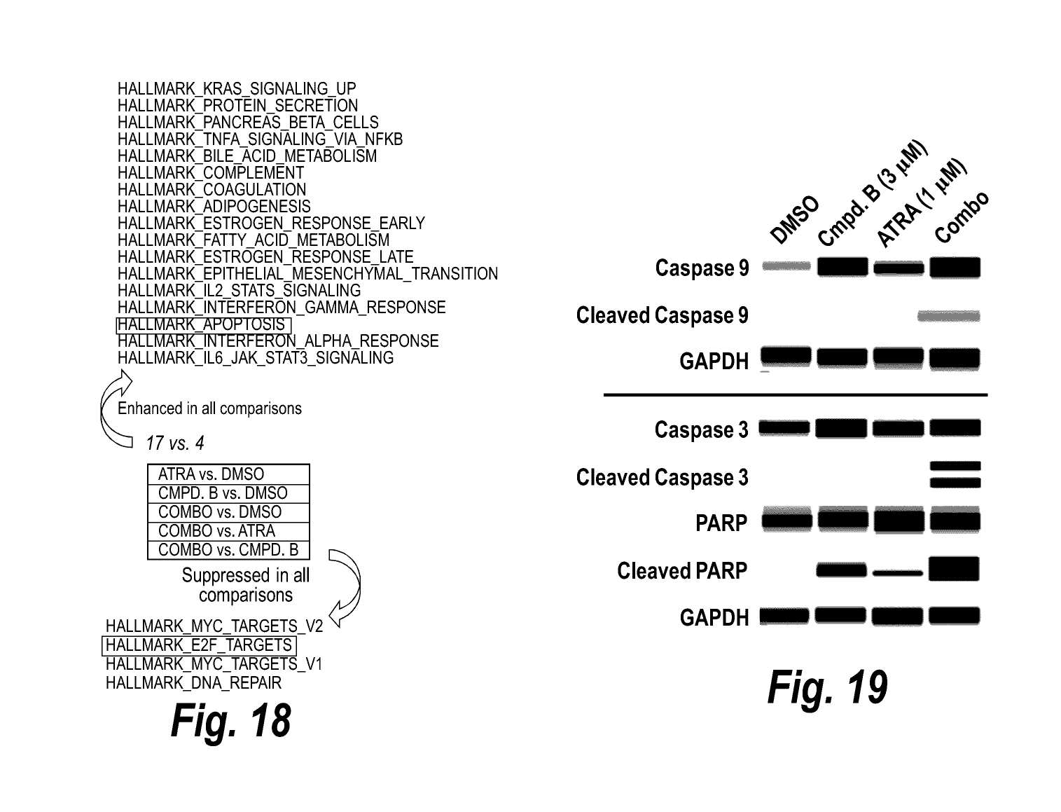

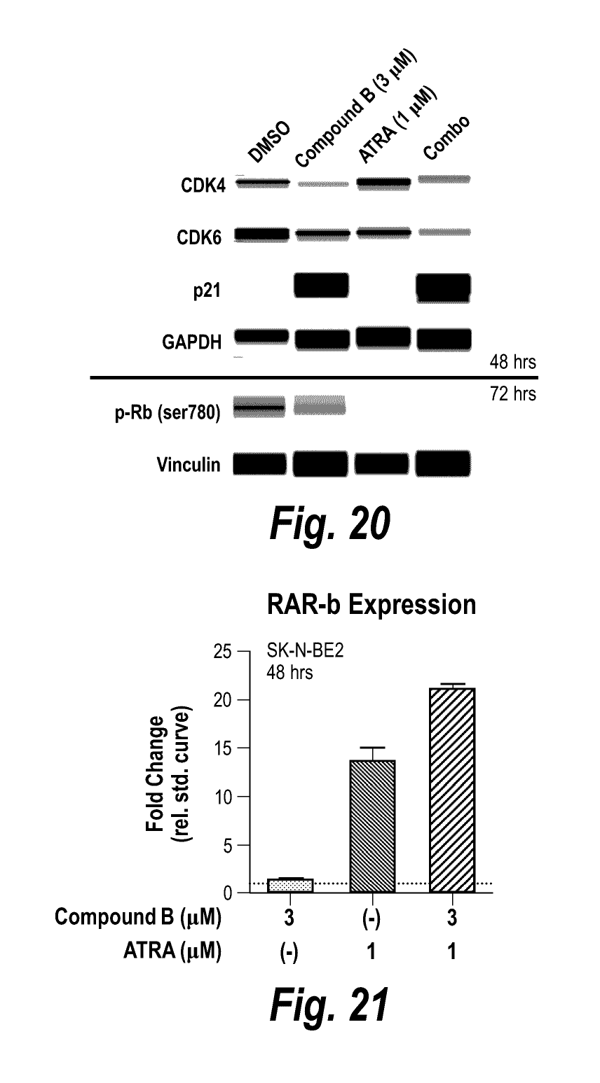

Gene set enrichment analysis using gene expression profiles from HDAC1/2i-treated cells and the Hallmark gene sets of the Molecular Signatures Database (MSigDB, Broad Institute) revealed a list of pathways that were consistently enriched by HDAC1/2i (FIG. 18). Pathways of KRAS signaling, protein secretion, pancreas beta cells, TNFA signaling via NFKB, bile acid metabolism, complement, coagulation, adipogenesis, estrogen response (early), fatty acid metabolism, estrogen response (late), epithelial mesenchymal transition, IL2-STATS signaling, interferon gamma response, apoptosis, interferon alpha response, and IL6-JAK-STAT3 signaling were enriched in genes up-regulated by HDAC1/2i either alone or in combination with RA, whereas pathways of MYC target (V2), E2F target (a proxy for cell cycle progression), MYC target (V1), and DNA repair were enriched in genes down-regulated by HDAC1/2i alone or in combination with RA. Caspase activation was examined and it was found that HDAC1/2i+RA enhanced cleavage of caspase 3 & 9 and PARP, consistent with apoptotic death (FIG. 19). When proteins involved in the E2F signaling pathway were examined, it was found that the pro-E2F signaling proteins CDK4 & 6 were decreased in the combination while the inhibitory protein p21 was increased (FIG. 20). Further, retinoblastoma (Rb) protein, a key regulator of the E2F transcription factor, was hypo-phosphorylated, suggesting E2F is in a repressed state.

### Microarray Experimental Design

Neuroblastoma cell line SK-N-BE(2) cells were treated with 3 μM Compound B, 1 μM ATRA, or a combination of both at 37° C. over 2 hours (FIG. 12A) or 48 hours (FIG. 12B) and compared to the solvent (DMSO) control.

TABLE 2
Pathways enriched in the 48 hr ATRA treated group relative to the combination setting

| NAME | Description | Set Size | NES | FDR q-val |
|---|---|---|---|---|
| PDGF_ERK_DN.V1_DN | ERK inactivation by inhibitors | 145 | 1.66 | 0.07 |
| WNT_UP.V1_UP | WNT1 overexpression | 176 | 1.48 | 0.11 |
| MYC_UP.V1_UP | MYC overexpression | 170 | 1.40 | 0.21 |
| HOXA9_DN.V1_DN | Genes decreased after HOXA9 knockdown | 184 | 1.39 | 0.18 |
| GCNP_SHH_UP_LATE.V1_UP | SHH stimulation in neuron precursors | 170 | 1.36 | 0.17 |
| GCNP_SHH_UP_EARLY.V1_UP | SHH stimulation in neuron precursors | 170 | 1.29 | 0.17 |
| AKT_UP_MTOR_DN.V1_DN | Genes decreased after AKT1 overexpression | 180 | 1.28 | 0.16 |
| CYCLIN_D1_UP.V1_UP | Overexpression of Cyclin D1 | 184 | 1.26 | 0.19 |

TABLE 3

| FC (abs) | Regulation | | |
|---|---|---|---|
| (Moderated | (Moderated | | |
| T-Test | T-Test | | |
| [Compound B] | [Compound B] | | |
| Vs [DMSO] | Vs [DMSO] | | |
| P <= 0.05 | P <= 0.05 | | |
| FC >= 2.0) | FC >= 2.0) | Symbol | Definition |
| 2.5698965 | up | TLE4 | Homo sapiens transducin-like |
| | | | enhancer of split 4 (E(sp1) homolog, |
| | | | Drosophila) (TLE4), mRNA. |
| 2.5064785 | up | STC1 | Homo sapiens stanniocalcin 1 |
| | | | (STC1), mRNA. |
| 2.3753035 | up | RALGPS1 | Homo sapiens Ral GEF with PH |
| | | | domain and SH3 binding motif 1 |
| | | | (RALGPS1), mRNA. |
| 2.2640777 | up | ARC | Homo sapiens activity-regulated |
| | | | cytoskeleton-associated protein |
| | | | (ARC), mRNA. |
| 2.2220922 | up | FAM83D | Homo sapiens family with sequence |
| | | | similarity 83, member D (FAM83D), |
| | | | mRNA. |
| 2.1907196 | up | RASD2 | Homo sapiens RASD family, |
| | | | member 2 (RASD2), mRNA. |
| 2.1847532 | up | RHOB | Homo sapiens ras homolog gene |
| | | | family, member B (RHOB), mRNA. |
| 2.1649334 | up | C5orf37 | Homo sapiens chromosome 5 open |
| | | | reading frame 37 (C5orf37), mRNA. |
| 2.143248 | up | NOTCH3 | Homo sapiens Notch homolog 3 |
| | | | (Drosophila) (NOTCH3), mRNA. |
| 2.1351867 | up | SRBD1 | Homo sapiens S1 RNA binding |
| | | | domain 1 (SRBD1), mRNA. |
| 2.1036422 | up | OLFML2A | Homo sapiens olfactomedin-like 2A |
| | | | (OLFML2A), mRNA. |
| 2.0986614 | up | CKS2 | Homo sapiens CDC28 protein kinase |
| | | | regulatory subunit 2 (CKS2), mRNA. |
| 2.0979898 | up | NEK2 | Homo sapiens NIMA (never in |
| | | | mitosis gene a)-related kinase 2 |
| | | | (NEK2), mRNA. |
| 2.0797677 | up | SULF2 | Homo sapiens sulfatase 2 (SULF2), |
| | | | transcript variant 1, mRNA. |
| 2.071442 | up | DPM1 | Homo sapiens dolichyl-phosphate |
| | | | mannosyltransferase polypeptide 1, |
| | | | catalytic subunit (DPM1), mRNA. |
| 2.065949 | up | | BX088936 Soares_testis_NHT |
| | | | Homo sapiens cDNA clone |
| | | | IMAGp998G123255; |
| | | | IMAGE: 1292195, mRNA sequence |
| 2.0578866 | up | FGFR3 | Homo sapiens fibroblast growth |
| | | | factor receptor 3 (achondroplasia, |
| | | | thanatophoric dwarfism) (FGFR3), |
| | | | transcript variant 2, mRNA. |
| 2.0553508 | up | CPNE4 | Homo sapiens copine IV (CPNE4), |
| | | | mRNA. |
| 2.0535789 | up | RGS5 | Homo sapiens regulator of G-protein |
| | | | signaling 5 (RGS5), mRNA. |
| 2.0502908 | up | PPM1E | Homo sapiens protein phosphatase |
| | | | 1E (PP2C domain containing) |
| | | | (PPM1E), mRNA. |
| 2.0453603 | up | FAM149B1 | Homo sapiens family with sequence |
| | | | similarity 149, member B1 |
| | | | (FAM149B1), mRNA. |
| 2.0264914 | up | ARL6IP1 | Homo sapiens ADP-ribosylation |
| | | | factor-like 6 interacting protein 1 |
| | | | (ARL6IP1), mRNA. |
| 2.0142202 | up | FGFR4 | Homo sapiens fibroblast growth |
| | | | factor receptor 4 (FGFR4), transcript |
| | | | variant 3, mRNA. |
| 2.0096514 | up | RPS6KA2 | Homo sapiens ribosomal protein S6 |
| | | | kinase, 90 kDa, polypeptide 2 |
| | | | (RPS6KA2), transcript variant 2, |
| | | | mRNA. |
| 2.2990463 | down | GAL | Homo sapiens galanin prepropeptide |
| | | | (GAL), mRNA. |
| 2.163846 | down | SHISA5 | Homo sapiens shisa homolog 5 |
| | | | (Xenopus laevis) (SHISA5), mRNA. |
| 2.104913 | down | TXNDC5 | Homo sapiens thioredoxin domain |
| | | | containing 5 (endoplasmic reticulum) |
| | | | (TXNDC5), transcript variant 1, |
| | | | mRNA. |
| 2.0841973 | down | SHD | Homo sapiens Src homology 2 |
| | | | domain containing transforming |
| | | | protein D (SHD), mRNA. |
| 2.0804224 | down | PRNP | Homo sapiens prion protein (PRNP), |
| | | | transcript variant 2, mRNA. |
| 2.0670848 | down | CPXM1 | Homo sapiens carboxypeptidase X |
| | | | (M14 family), member 1 (CPXM1), |
| | | | mRNA. |
| 2.0438597 | down | ROBO2 | Homo sapiens roundabout, axon |
| | | | guidance receptor, homolog 2 |
| | | | (Drosophila) (ROBO2), mRNA. |
| 2.0305886 | down | PCGF1 | Homo sapiens polycomb group ring |
| | | | finger 1 (PCGF1), mRNA. |
| 2.0155308 | down | LDLR | Homo sapiens low density |
| | | | lipoprotein receptor (familial |
| | | | hypercholesterolemia) (LDLR), |
| | | | mRNA. |
| 2.0127265 | down | CD151 | Homo sapiens CD151 molecule |
| | | | (Raph blood group) (CD151), |
| | | | transcript variant 2, mRNA. |

TABLE 4

| FC (abs) | Regulation | | |
|---|---|---|---|
| (Moderated | (Moderated | | |
| T-Test | T-Test | | |
| [Compound B] | [Compound B] | | |
| Vs [DMSO] | Vs [DMSO] | | |
| P <= 0.05 | P <= 0.05 | Gene | |
| FC >= 2.0) | FC >= 2.0) | Symbol | Definition |
| 2.286589 | up | BNIP3 | Homo sapiens |
| | | | BCL2/adenovirus E1B |
| | | | 19 kDa interacting protein 3 |
| | | | (BNIP3), nuclear gene |
| | | | encoding mitochondrial |
| | | | protein, mRNA. |
| 2.023285 | up | RGS4 | Homo sapiens |
| | | | regulator of G-protein |
| | | | signalling 4 (RGS4), |
| | | | mRNA. |
| 2.220195 | down | FAM163A | Homo sapiens |
| | | | family with sequence |
| | | | similarity 163, member A |
| | | | (FAM163A), mRNA. |
| 2.08247 | down | FAM163A | Homo sapiens |
| | | | family with sequence |
| | | | similarity 163, member A |
| | | | (FAM163A), mRNA. |

TABLE 5

| FC (abs) | Regulation | | |
|---|---|---|---|
| (Moderated | (Moderated | | |
| T-Test | T-Test | | |
| [ATRA] | [ATRA] | | |
| Vs [DMSO] | Vs [DMSO] | | |
| P <= 0.05 | P <= 0.05 | | |
| FC >= 2.0) | FC >= 2.0) | Symbol | Definition |
| 8.72177 | up | GDF10 | Homo sapiens growth differentiation |
| | | | factor 10 (GDF10), mRNA. |
| 4.283083 | up | DKK2 | Homo sapiens dickkopf homolog 2 |
| | | | (Xenopus laevis) (DKK2), mRNA. |
| 3.677953 | up | TNS3 | Homo sapiens tensin 3 (TNS3), |
| | | | mRNA. |
| 3.677291 | up | TLX2 | Homo sapiens T-cell leukemia |
| | | | homeobox 2 (TLX2), mRNA. |
| 3.319324 | up | NTRK2 | Homo sapiens neurotrophic tyrosine |
| | | | kinase, receptor, type 2 (NTRK2), |
| | | | transcript variant b, mRNA. |
| 3.18521 | up | ADAMTS9 | Homo sapiens ADAM |
| | | | metallopeptidase with |
| | | | thrombospondin type 1 motif, 9 |
| | | | (ADAMTS9), mRNA. |
| 2.99289 | up | TSHZ3 | Homo sapiens teashirt zinc finger |
| | | | homeobox 3 (TSHZ3), mRNA. |
| 2.986171 | up | RBP1 | Homo sapiens retinol binding protein |
| | | | 1, cellular (RBP1), mRNA. |
| 2.937687 | up | DYSF | Homo sapiens dysferlin, limb girdle |
| | | | muscular dystrophy 2B (autosomal |
| | | | recessive) (DYSF), mRNA. |
| 2.80848 | up | DICER1 | Homo sapiens Dicer1, Dcr-1 homolog |
| | | | (Drosophila) (DICER1), transcript |
| | | | variant 2, mRNA. |
| 2.800005 | up | C20orf103 | Homo sapiens chromosome 20 open |
| | | | reading frame 103 (C20orf103), |
| | | | mRNA. |
| 2.795654 | up | SLCO2A1 | Homo sapiens solute carrier organic |
| | | | anion transporter family, member 2A1 |
| | | | (SLCO2A1), mRNA. |
| 2.722439 | up | DKK2 | Homo sapiens dickkopf homolog 2 |
| | | | (Xenopus laevis) (DKK2), mRNA. |
| 2.714301 | up | SCHIP1 | Homo sapiens schwannomin |
| | | | interacting protein 1 (SCHIP1), |
| | | | mRNA. |
| 2.66298 | up | ARHGAP28 | Homo sapiens Rho GTPase activating |
| | | | protein 28 (ARHGAP28), transcript |
| | | | variant 1, mRNA. |
| 2.651171 | up | YPEL2 | Homo sapiens yippee-like 2 |
| | | | (Drosophila) (YPEL2), mRNA. |
| 2.644729 | up | SNORA59A | Homo sapiens small nucleolar RNA, |
| | | | H/ACA box 59A (SNORA59A), small |
| | | | nucleolar RNA. |
| 2.596732 | up | DICER1 | Homo sapiens Dicer1, Dcr-1 homolog |
| | | | (Drosophila) (DICER1), transcript |
| | | | variant 2, mRNA. |
| 2.565 | up | VPS13D | Homo sapiens vacuolar protein sorting |
| | | | 13 homolog D (S. cerevisiae) |
| | | | (VPS13D), transcript variant 1, |
| | | | mRNA. |
| 2.547531 | up | NR0B1 | Homo sapiens nuclear receptor |
| | | | subfamily 0, group B, member 1 |
| | | | (NR0B1), mRNA. |
| 2.490577 | up | C15orf52 | Homo sapiens chromosome 15 open |
| | | | reading frame 52 (C15orf52), mRNA. |
| 2.455256 | up | HOXD8 | Homo sapiens homeobox D8 |
| | | | (HOXD8), mRNA. |
| 2.452022 | up | TBX3 | Homo sapiens T-box 3 (TBX3), |
| | | | transcript variant 1, mRNA. |
| 2.399388 | up | ARHGAP28 | Homo sapiens Rho GTPase activating |
| | | | protein 28 (ARHGAP28), transcript |
| | | | variant 1, mRNA. |
| 2.388347 | up | CCND1 | Homo sapiens cyclin D1 (CCND1), |
| | | | mRNA. |
| 2.382721 | up | KIAA1641 | PREDICTED: Homo sapiens |
| | | | KIAA1641, transcript variant 7 |
| | | | (KIAA1641), mRNA. |
| 2.380837 | up | TEX101 | Homo sapiens testis expressed 101 |
| | | | (TEX101), mRNA. |
| 2.364498 | up | HERC2P2 | Homo sapiens hect domain and RLD 2 |
| | | | pseudogene 2 (HERC2P2) on |
| | | | chromosome 15. |
| 2.329918 | up | ZBED5 | Homo sapiens zinc finger, BED-type |
| | | | containing 5 (ZBED5), mRNA. |
| 2.324694 | up | MPPED2 | Homo sapiens metallophosphoesterase |
| | | | domain containing 2 (MPPED2), |
| | | | mRNA. |
| 2.311865 | up | LOC642530 | PREDICTED: Homo sapiens |
| | | | hypothetical protein LOC642530 |
| | | | (LOC642530), mRNA. |
| 2.293098 | up | HOXD3 | Homo sapiens homeobox D3 |
| | | | (HOXD3), mRNA. |
| 2.275352 | up | LHFPL2 | Homo sapiens lipoma HMGIC fusion |
| | | | partner-like 2 (LHFPL2), mRNA. |
| 2.255434 | up | | PREDICTED: Homo sapiens |
| | | | hypothetical LOC400043 |
| | | | (LOC400043), mRNA |
| 2.207207 | up | | Homo sapiens cDNA: FLJ21199 fis, |
| | | | clone COL00235 |
| 2.203424 | up | ZBED5 | Homo sapiens zinc finger, BED-type |
| | | | containing 5 (ZBED5), mRNA. |
| 2.197959 | up | SGIP1 | Homo sapiens SH3-domain GRB2- |
| | | | like (endophilin) interacting protein 1 |
| | | | (SGIP1), mRNA. |
| 2.196478 | up | ANTXR1 | Homo sapiens anthrax toxin receptor 1 |
| | | | (ANTXR1), transcript variant 1, |
| | | | mRNA. |
| 2.184386 | up | BHLHB2 | Homo sapiens basic helix-loop-helix |
| | | | domain containing, class B, 2 |
| | | | (BHLHB2), mRNA. |
| 2.181258 | up | LBX2 | Homo sapiens ladybird homeobox 2 |
| | | | (LBX2), mRNA. |
| 2.174702 | up | HNRPM | Homo sapiens heterogeneous nuclear |
| | | | ribonucleoprotein M (HNRPM), |
| | | | transcript variant 1, mRNA. |
| 2.171866 | up | PPP1R10 | Homo sapiens protein phosphatase 1, |
| | | | regulatory (inhibitor) subunit 10 |
| | | | (PPP1R10), mRNA. |
| 2.157885 | up | HNRNPM | Homo sapiens heterogeneous nuclear |
| | | | ribonucleoprotein M (HNRNPM), |
| | | | transcript variant 1, mRNA. |
| 2.133766 | up | LIMA1 | Homo sapiens LIM domain and actin |
| | | | binding 1 (LIMA1), mRNA. |
| 2.12906 | up | C4orf6 | Homo sapiens chromosome 4 open |
| | | | reading frame 6 (C4orf6), mRNA. |
| 2.116109 | up | HEBP2 | Homo sapiens heme binding protein 2 |
| | | | (HEBP2), mRNA. |
| 2.108607 | up | SEMA5A | Homo sapiens sema domain, seven |
| | | | thrombospondin repeats (type 1 and |
| | | | type 1-like), transmembrane domain |
| | | | (TM) and short cytoplasmic domain, |
| | | | (semaphorin) 5A (SEMA5A), mRNA. |
| 2.107324 | up | AJAP1 | Homo sapiens adherens junctions |
| | | | associated protein 1 (AJAP1), |
| | | | transcript variant 2, mRNA. |
| 2.106345 | up | DCX | Homo sapiens doublecortin (DCX), |
| | | | transcript variant 4, mRNA. |
| 2.094997 | up | POPDC2 | Homo sapiens popeye domain |
| | | | containing 2 (POPDC2), mRNA. |
| 2.092158 | up | TTC30B | Homo sapiens tetratricopeptide repeat |
| | | | domain 30B (TTC30B), mRNA. |
| 2.087778 | up | CYTL1 | Homo sapiens cytokine-like 1 |
| | | | (CYTL1), mRNA. |
| 2.087406 | up | DLGAP5 | Homo sapiens discs, large |
| | | | (Drosophila) homolog-associated |
| | | | protein 5 (DLGAP5), mRNA. |
| 2.057107 | up | AKAP12 | Homo sapiens A kinase (PRKA) |
| | | | anchor protein (gravin) 12 (AKAP12), |
| | | | transcript variant 1, mRNA. |
| 2.052444 | up | EIF4G2 | Homo sapiens eukaryotic translation |
| | | | initiation factor 4 gamma, 2 (EIF4G2), |
| | | | transcript variant 1, mRNA. |
| 2.045954 | up | FAM40A | Homo sapiens family with sequence |
| | | | similarity 40, member A (FAM40A), |
| | | | mRNA. |
| 2.042676 | up | HNRNPM | Homo sapiens heterogeneous nuclear |
| | | | ribonucleoprotein M (HNRNPM), |
| | | | transcript variant 2, mRNA. |
| 2.038353 | up | PPARG | Homo sapiens peroxisome |
| | | | proliferator-activated receptor gamma |
| | | | (PPARG), transcript variant 2, mRNA. |
| 2.032579 | up | SNAI2 | Homo sapiens snail homolog 2 |
| | | | (Drosophila) (SNAI2), mRNA. |
| 2.015555 | up | GMPPA | Homo sapiens GDP-mannose |
| | | | pyrophosphorylase A (GMPPA), |
| | | | transcript variant 2, mRNA. |
| 2.014441 | up | TM4SF4 | Homo sapiens transmembrane 4 L six |
| | | | family member 4 (TM4SF4), mRNA. |
| 2.378933 | down | KAL1 | Homo sapiens Kallmann syndrome 1 |
| | | | sequence (KAL1), mRNA. |
| 2.103935 | down | SC4MOL | Homo sapiens sterol-C4-methyl |
| | | | oxidase-like (SC4MOL), transcript |
| | | | variant 1, mRNA. |
| 2.073776 | down | VIM | Homo sapiens vimentin (VIM), |
| | | | mRNA. |
| 2.070505 | down | RPL9 | Homo sapiens ribosomal protein L9 |
| | | | (RPL9), transcript variant 2, mRNA. |
| 2.048986 | down | RPL37 | Homo sapiens ribosomal protein L37 |
| | | | (RPL37), mRNA. |
| 2.036535 | down | LOC399491 | PREDICTED: Homo sapiens |
| | | | misc_RNA (LOC399491), miscRNA. |
| 2.033366 | down | PRSS12 | Homo sapiens protease, serine, 12 |
| | | | (neurotrypsin, motopsin) (PRSS12), |
| | | | mRNA. |
| 2.029949 | down | ADAM15 | Homo sapiens ADAM |
| | | | metallopeptidase domain 15 |
| | | | (ADAM15), transcript variant 2, |
| | | | mRNA. |
| 2.029488 | down | LAMA5 | Homo sapiens laminin, alpha 5 |
| | | | (LAMA5), mRNA. |
| 2.016979 | down | PDXP | Homo sapiens pyridoxal (pyridoxine, |
| | | | vitamin B6) phosphatase (PDXP), |
| | | | mRNA. |

TABLE 6

| FC (abs) | Regulation | | |
|---|---|---|---|
| (Moderated | (Moderated | | |
| T-Test | T-Test | | |
| [Compound B] | [Compound B] | | |
| Vs [DMSO] | Vs [DMSO] | | |
| P <= 0.05 | P <= 0.05 | | |
| FC >= 2.0) | FC >= 2.0) | Symbol | Definition |
| 2.120727 | down | CD44 | Homo sapiens CD44 molecule (Indian |
| | | | blood group) (CD44), transcript |
| | | | variant 4, mRNA. |
| 2.416998 | down | SNORA8 | Homo sapiens small nucleolar RNA, |
| | | | H/ACA box 8 (SNORA8), small |
| | | | nucleolar RNA. |
| 2.51326 | down | CDH24 | Homo sapiens cadherin-like 24 |
| | | | (CDH24), transcript variant 1, mRNA. |
| 2.641853 | down | DLK1 | Homo sapiens delta-like 1 homolog |
| | | | (Drosophila) (DLK1), mRNA. |
| 2.737174 | down | PTCHD1 | Homo sapiens patched domain |
| | | | containing 1 (PTCHD1), mRNA. |
| 3.346026 | down | SLC6A15 | Homo sapiens solute carrier family 6 |
| | | | (neutral amino acid transporter), |
| | | | member 15 (SLC6A15), transcript |
| | | | variant 2, mRNA. |
| 4.589464 | down | STMN4 | Homo sapiens stathmin-like 4 |
| | | | (STMN4), mRNA. |
| 2.028951 | up | BMP4 | Homo sapiens bone morphogenetic |
| | | | protein 4 (BMP4), transcript variant 3, |
| | | | mRNA. |
| 2.07585 | up | RGS16 | Homo sapiens regulator of G-protein |
| | | | signalling 16 (RGS16), mRNA. |
| 2.293513 | up | IER3 | Homo sapiens immediate early |
| | | | response 3 (IER3), mRNA. |
| 2.303625 | up | RGL1 | Homo sapiens ral guanine nucleotide |
| | | | dissociation stimulator-like 1 (RGL1), |
| | | | mRNA. |
| 2.430483 | up | SGK | Homo sapiens serum/glucocorticoid |
| | | | regulated kinase (SGK), mRNA. |
| 2.749795 | up | | Homo sapiens mRNA full length |
| | | | insert cDNA clone EUROIMAGE |
| | | | 151432 |
| 3.030697 | up | CTSH | Homo sapiens cathepsin H (CTSH), |
| | | | transcript variant 1, mRNA. |
| 3.177024 | up | ETS1 | Homo sapiens v-ets erythroblastosis |
| | | | virus E26 oncogene homolog 1 (avian) |
| | | | (ETS1), mRNA. |
| 3.274463 | up | ETS1 | Homo sapiens v-ets erythroblastosis |
| | | | virus E26 oncogene homolog 1 (avian) |
| | | | (ETS1), mRNA. |
| 3.331427 | up | DUSP6 | Homo sapiens dual specificity |
| | | | phosphatase 6 (DUSP6), transcript |
| | | | variant 2, mRNA. |
| 3.41655 | up | SIPA1L2 | Homo sapiens signal-induced |
| | | | proliferation-associated 1 like 2 |
| | | | (SIPA1L2), mRNA. |
| 4.000809 | up | EGR1 | Homo sapiens early growth response 1 |
| | | | (EGR1), mRNA. |
| 7.405237 | up | FOS | Homo sapiens v-fos FBJ murine |
| | | | osteosarcoma viral oncogene homolog |
| | | | (FOS), mRNA. |

TABLE 7

| FC (abs) | Regulation | | |
|---|---|---|---|
| (Moderated | (Moderated | | |
| T-Test | T-Test | | |
| [Compound B] | [Compound B] | | |
| Vs [DMSO] | Vs [DMSO] | | |
| P <= 0.05 | P <= 0.05 | Gene | |
| FC >= 2.0) | FC >= 2.0) | Symbol | Definition |
| 3.7700295 | up | HSPA5 | Homo sapiens heat shock 70 kDa |
| | | | protein 5 (glucose-regulated protein, |
| | | | 78 kDa) (HSPA5), mRNA. |
| 3.463821 | up | NPC2 | Homo sapiens Niemann-Pick |
| | | | disease, type C2 (NPC2), mRNA. |
| 3.382726 | up | PQLC3 | Homo sapiens PQ loop repeat |
| | | | containing 3 (PQLC3), mRNA. |
| 3.2470052 | up | CFD | Homo sapiens complement factor D |
| | | | (adipsin) (CFD), mRNA. |
| 3.2319236 | up | DHRS2 | Homo sapiens |
| | | | dehydrogenase/reductase (SDR |
| | | | family) member 2 (DHRS2), |
| | | | transcript variant 1, mRNA. |
| 3.1507273 | up | POU4F1 | Homo sapiens POU class 4 |
| | | | homeobox 1 (POU4F1), mRNA. |
| 3.0529962 | up | MYLIP | Homo sapiens myosin regulatory |
| | | | light chain interacting protein |
| | | | (MYLIP), mRNA. |
| 3.006488 | up | AIF1L | Homo sapiens allograft inflammatory |
| | | | factor 1-like (AIF1L), transcript |
| | | | variant 1, mRNA. |
| 2.889204 | up | | AGENCOURT_10229596 |
| | | | NIH_MGC_141 Homo sapiens |
| | | | cDNA clone IMAGE: 6563923 5, |
| | | | mRNA sequence |
| 2.848912 | up | HMMR | Homo sapiens hyaluronan-mediated |
| | | | motility receptor (RHAMM) |
| | | | (HMMR), transcript variant 2, |
| | | | mRNA. |
| 2.7428732 | up | SCPEP1 | Homo sapiens serine |
| | | | carboxypeptidase 1 (SCPEP1), |
| | | | mRNA. |
| 2.741772 | up | MERTK | Homo sapiens c-mer proto-oncogene |
| | | | tyrosine kinase (MERTK), mRNA. |
| 2.7384179 | up | LOC338758 | PREDICTED: Homo sapiens |
| | | | hypothetical protein LOC338758 |
| | | | (LOC338758), mRNA. |
| 2.7313623 | up | CIB1 | Homo sapiens calcium and integrin |
| | | | binding 1 (calmyrin) (CIB1), mRNA. |
| 2.7183042 | up | COL5A1 | Homo sapiens collagen, type V, |
| | | | alpha 1 (COL5A1), mRNA. |
| 2.6359165 | up | CTSL2 | Homo sapiens cathepsin L2 |
| | | | (CTSL2), mRNA. |
| 2.6211162 | up | IFI6 | Homo sapiens interferon, alpha- |
| | | | inducible protein 6 (IFI6), transcript |
| | | | variant 3, mRNA. |
| 2.6199353 | up | CGN | Homo sapiens cingulin (CGN), |
| | | | mRNA. |
| 2.6197765 | up | CPVL | Homo sapiens carboxypeptidase, |
| | | | vitellogenic-like (CPVL), transcript |
| | | | variant 1, mRNA. |
| 2.6172824 | up | PPP2R2B | Homo sapiens protein phosphatase 2 |
| | | | (formerly 2A), regulatory subunit B, |
| | | | beta isoform (PPP2R2B), transcript |
| | | | variant 4, mRNA. |
| 2.613672 | up | CCDC99 | Homo sapiens coiled-coil domain |
| | | | containing 99 (CCDC99), mRNA. |
| 2.5982802 | up | CYP2J2 | Homo sapiens cytochrome P450, |
| | | | family 2, subfamily J, polypeptide 2 |
| | | | (CYP2J2), mRNA. |
| 2.5876853 | up | BAMBI | Homo sapiens BMP and activin |
| | | | membrane-bound inhibitor homolog |
| | | | (Xenopus laevis) (BAMBI), mRNA. |
| 2.5142672 | up | HSPA1A | Homo sapiens heat shock 70 kDa |
| | | | protein 1A (HSPA1A), mRNA. |
| 2.5097656 | up | RN7SK | Homo sapiens RNA, 7SK small |
| | | | nuclear (RN7SK), non-coding RNA. |
| 2.4892557 | up | ITPR1 | Homo sapiens inositol 1,4,5- |
| | | | triphosphate receptor, type 1 |
| | | | (ITPR1), transcript variant 2, mRNA. |
| 2.4447675 | up | SPA17 | Homo sapiens sperm autoantigenic |
| | | | protein 17 (SPA17), mRNA. |
| 2.4395416 | up | ESRRG | Homo sapiens estrogen-related |
| | | | receptor gamma (ESRRG), transcript |
| | | | variant 2, mRNA. |
| 2.4309964 | up | CLDN11 | Homo sapiens claudin 11 |
| | | | (oligodendrocyte transmembrane |
| | | | protein) (CLDN11), mRNA. |
| 2.4051182 | up | ST6GALNAC3 | Homo sapiens ST6 (alpha-N-acetyl- |
| | | | neuraminyl-2,3-beta-galactosyl-1,3)- |
| | | | N-acetylgalactosaminide alpha-2,6- |
| | | | sialyltransferase 3 (ST6GALNAC3), |
| | | | mRNA. |
| 2.3992615 | up | STAT1 | Homo sapiens signal transducer and |
| | | | activator of transcription 1, 91 kDa |
| | | | (STAT1), transcript variant alpha, |
| | | | mRNA. |
| 2.382162 | up | PPP1R3C | Homo sapiens protein phosphatase 1, |
| | | | regulatory (inhibitor) subunit 3C |
| | | | (PPP1R3C), mRNA. |
| 2.3813944 | up | CRY1 | Homo sapiens cryptochrome 1 |
| | | | (photolyase-like) (CRY1), mRNA. |
| 2.3754704 | up | RYBP | Homo sapiens RING1 and YY1 |
| | | | binding protein (RYBP), mRNA. |
| 2.365169 | up | FSTL5 | Homo sapiens follistatin-like 5 |
| | | | (FSTL5), mRNA. |
| 2.316434 | up | PRSS35 | Homo sapiens protease, serine, 35 |
| | | | (PRSS35), mRNA. |
| 2.3060548 | up | SERPINE2 | Homo sapiens serpin peptidase |
| | | | inhibitor, clade E (nexin, |
| | | | plasminogen activator inhibitor type |
| | | | 1), member 2 (SERPINE2), mRNA. |
| 2.3006706 | up | HMMR | Homo sapiens hyaluronan-mediated |
| | | | motility receptor (RHAMM) |
| | | | (HMMR), transcript variant 1, |
| | | | mRNA. |
| 2.2822168 | up | GLRX | Homo sapiens glutaredoxin |
| | | | (thioltransferase) (GLRX), mRNA. |
| 2.271626 | up | LMO4 | Homo sapiens LIM domain only 4 |
| | | | (LMO4), mRNA. |
| 2.2702909 | up | IL13RA2 | Homo sapiens interleukin 13 |
| | | | receptor, alpha 2 (IL13RA2), |
| | | | mRNA. |
| 2.269596 | up | IGSF3 | Homo sapiens immunoglobulin |
| | | | superfamily, member 3 (IGSF3), |
| | | | transcript variant 1, mRNA. |
| 2.257454 | up | NEK1 | Homo sapiens NIMA (never in |
| | | | mitosis gene a)-related kinase 1 |
| | | | (NEK1), mRNA. |
| 2.243685 | up | CAST | Homo sapiens calpastatin (CAST), |
| | | | transcript variant 8, mRNA. |
| 2.223723 | up | PAG1 | Homo sapiens phosphoprotein |
| | | | associated with glycosphingolipid |
| | | | microdomains 1 (PAG1), mRNA. |
| 2.2132776 | up | STK3 | Homo sapiens serine/threonine |
| | | | kinase 3 (STE20 homolog, yeast) |
| | | | (STK3), mRNA. |
| 2.211524 | up | NPTX2 | Homo sapiens neuronal pentraxin II |
| | | | (NPTX2), mRNA. |
| 2.2004776 | up | CAP1 | Homo sapiens CAP, adenylate |
| | | | cyclase-associated protein 1 (yeast) |
| | | | (CAP1), mRNA. |
| 2.1915953 | up | HSPA2 | Homo sapiens heat shock 70 kDa |
| | | | protein 2 (HSPA2), mRNA. |
| 2.1893888 | up | SDF2L1 | Homo sapiens stromal cell-derived |
| | | | factor 2-like 1 (SDF2L1), mRNA. |
| 2.1769004 | up | ACO1 | Homo sapiens aconitase 1, soluble |
| | | | (ACO1), mRNA. |
| 2.166546 | up | MAP4K2 | Homo sapiens mitogen-activated |
| | | | protein kinase kinase kinase kinase 2 |
| | | | (MAP4K2), mRNA. |
| 2.1538491 | up | CRYZ | Homo sapiens crystallin, zeta |
| | | | (quinone reductase) (CRYZ), |
| | | | mRNA. |
| 2.1500034 | up | DNCL1 | Homo sapiens dynein, cytoplasmic, |
| | | | light polypeptide 1 (DNCL1), |
| | | | mRNA. |
| 2.1493442 | up | CREG1 | Homo sapiens cellular repressor of |
| | | | E1A-stimulated genes 1 (CREG1), |
| | | | mRNA. |
| 2.1460152 | up | RHBDF2 | Homo sapiens rhomboid 5 homolog |
| | | | 2 (Drosophila) (RHBDF2), transcript |
| | | | variant 2, mRNA. |
| 2.145611 | up | | Homo sapiens cDNA FLJ43160 fis, |
| | | | clone FCBBF2000199 |
| 2.1441228 | up | PYGL | Homo sapiens phosphorylase, |
| | | | glycogen, liver (PYGL), mRNA. |
| 2.1436455 | up | LRRC1 | Homo sapiens leucine rich repeat |
| | | | containing 1 (LRRC1), mRNA. |
| 2.1423635 | up | LOC730432 | PREDICTED: Homo sapiens similar |
| | | | to serine/threonine/tyrosine |
| | | | interacting protein, transcript variant |
| | | | 1 (LOC730432), mRNA. |
| 2.132023 | up | SERPINI1 | Homo sapiens serpin peptidase |
| | | | inhibitor, clade I (neuroserpin), |
| | | | member 1 (SERPINI1), mRNA. |
| 2.1283884 | up | CBR4 | Homo sapiens carbonyl reductase 4 |
| | | | (CBR4), mRNA. |
| 2.1136534 | up | RAB23 | Homo sapiens RAB23, member RAS |
| | | | oncogene family (RAB23), transcript |
| | | | variant 1, mRNA. |
| 2.1128619 | up | VCL | Homo sapiens vinculin (VCL), |
| | | | transcript variant 1, mRNA. |
| 2.112098 | up | ETV5 | Homo sapiens ets variant gene 5 (ets- |
| | | | related molecule) (ETV5), mRNA. |
| 2.109667 | up | TIPARP | Homo sapiens TCDD-inducible |
| | | | poly(ADP-ribose) polymerase |
| | | | (TIPARP), mRNA. |
| 2.109442 | up | ALS2 | Homo sapiens amyotrophic lateral |
| | | | sclerosis 2 (juvenile) (ALS2), |
| | | | mRNA. |
| 2.0956025 | up | SDCBP | Homo sapiens syndecan binding |
| | | | protein (syntenin) (SDCBP), |
| | | | transcript variant 2, mRNA. |
| 2.0934463 | up | FERMT2 | Homo sapiens fermitin family |
| | | | homolog 2 (Drosophila) (FERMT2), |
| | | | mRNA. |
| 2.0914667 | up | TJP1 | Homo sapiens tight junction protein |
| | | | 1 (zona occludens 1) (TJP1), |
| | | | transcript variant 1, mRNA. |
| 2.088695 | up | POP5 | Homo sapiens processing of |
| | | | precursor 5, ribonuclease P/MRP |
| | | | subunit (S. cerevisiae) (POP5), |
| | | | transcript variant 3, mRNA. |
| 2.081764 | up | LCMT2 | Homo sapiens leucine carboxyl |
| | | | methyltransferase 2 (LCMT2), |
| | | | mRNA. |
| 2.05315 | up | CEP55 | Homo sapiens centrosomal protein |
| | | | 55 kDa (CEP55), mRNA. |
| 2.044371 | up | PLCB1 | Homo sapiens phospholipase C, beta |
| | | | 1 (phosphoinositide-specific) |
| | | | (PLCB1), transcript variant 1, |
| | | | mRNA. |
| 2.0366564 | up | KIAA1618 | Homo sapiens KIAA1618 |
| | | | (KIAA1618), mRNA. |
| 2.034572 | up | BCL2L12 | Homo sapiens BCL2-like 12 (proline |
| | | | rich) (BCL2L12), transcript variant |
| | | | 3, mRNA. |
| 2.0263453 | up | PDGFD | Homo sapiens platelet derived |
| | | | growth factor D (PDGFD), transcript |
| | | | variant 2, mRNA. |
| 2.0171542 | up | CDC14B | Homo sapiens CDC 14 cell division |
| | | | cycle 14 homolog B (S. cerevisiae) |
| | | | (CDC14B), transcript variant 2, |
| | | | mRNA. |
| 2.0131624 | up | CRELD2 | Homo sapiens cysteine-rich with |
| | | | EGF-like domains 2 (CRELD2), |
| | | | mRNA. |
| 2.0129623 | up | FLJ35767 | Homo sapiens FLJ35767 protein |
| | | | (FLJ35767), mRNA. |
| 2.0035446 | up | SCN9A | Homo sapiens sodium channel, |
| | | | voltage-gated, type IX, alpha subunit |
| | | | (SCN9A), mRNA. |
| 2.0030117 | up | LOC441089 | Homo sapiens CRSP8 pseudogene |
| | | | (LOC441089), non-coding RNA. |
| 2.0013416 | up | PLS1 | Homo sapiens plastin 1 (I isoform) |
| | | | (PLS1), mRNA. |
| 3.2073772 | down | MIAT | Homo sapiens myocardial infarction |
| | | | associated transcript (non-protein |
| | | | coding) (MIAT), non-coding RNA. |
| 3.134669 | down | C16orf53 | Homo sapiens chromosome 16 open |
| | | | reading frame 53 (C16orf53), |
| | | | mRNA. |
| 3.0669296 | down | PCOLCE | Homo sapiens procollagen C- |
| | | | endopeptidase enhancer (PCOLCE), |
| | | | mRNA. |
| 2.9874208 | down | TYMS | Homo sapiens thymidylate |
| | | | synthetase (TYMS), mRNA. |
| 2.9546156 | down | ASAM | Homo sapiens adipocyte-specific |
| | | | adhesion molecule (ASAM), mRNA. |
| 2.881155 | down | FLJ25404 | PREDICTED: Homo sapiens |
| | | | hypothetical protein FLJ25404, |
| | | | transcript variant 2 (FLJ25404), |
| | | | mRNA. |
| 2.7996004 | down | ICA1 | Homo sapiens islet cell autoantigen |
| | | | 1, 69 kDa (ICA1), transcript variant |
| | | | 2, mRNA. |
| 2.7547011 | down | SLC6A15 | Homo sapiens solute carrier family |
| | | | 6, member 15 (SLC6A15), transcript |
| | | | variant 1, mRNA. |
| 2.6473744 | down | DUSP26 | Homo sapiens dual specificity |
| | | | phosphatase 26 (putative) (DUSP26), |
| | | | mRNA. |
| 2.6301363 | down | SH2D3C | Homo sapiens SH2 domain |
| | | | containing 3C (SH2D3C), transcript |
| | | | variant 2, mRNA. |
| 2.6298933 | down | LRFN4 | Homo sapiens leucine rich repeat and |
| | | | fibronectin type III domain |
| | | | containing 4 (LRFN4), mRNA. |
| 2.5819318 | down | CENPV | Homo sapiens centromere protein V |
| | | | (CENPV), mRNA. |
| 2.569341 | down | DDX17 | Homo sapiens DEAD (Asp-Glu-Ala- |
| | | | Asp) box polypeptide 17 (DDX17), |
| | | | transcript variant 1, mRNA. |
| 2.5645072 | down | C16orf53 | Homo sapiens chromosome 16 open |
| | | | reading frame 53 (C16orf53), |
| | | | mRNA. |
| 2.5115252 | down | CLASP2 | Homo sapiens cytoplasmic linker |
| | | | associated protein 2 (CLASP2), |
| | | | mRNA. |
| 2.4085312 | down | ARMCX1 | Homo sapiens armadillo repeat |
| | | | containing, X-linked 1 (ARMCX1), |
| | | | mRNA. |
| 2.395616 | down | ICA1 | Homo sapiens islet cell autoantigen |
| | | | 1, 69 kDa (ICA1), transcript variant |
| | | | 2, mRNA. |
| 2.3802598 | down | LAMB1 | Homo sapiens laminin, beta 1 |
| | | | (LAMB1), mRNA. |
| 2.3713975 | down | CLK1 | Homo sapiens CDC-like kinase 1 |
| | | | (CLK1), mRNA. |
| 2.3289754 | down | TH | Homo sapiens tyrosine hydroxylase |
| | | | (TH), transcript variant 3, mRNA. |
| 2.3156798 | down | P4HTM | Homo sapiens prolyl 4-hydroxylase, |
| | | | transmembrane (endoplasmic |
| | | | reticulum) (P4HTM), transcript |
| | | | variant 3, mRNA. |
| 2.3148339 | down | D4S234E | Homo sapiens DNA segment on |
| | | | chromosome 4 (unique) 234 |
| | | | expressed sequence (D4S234E), |
| | | | transcript variant 2, mRNA. |
| 2.2813866 | down | MTA1 | Homo sapiens metastasis associated |
| | | | 1 (MTA1), mRNA. |
| 2.2809587 | down | TUB | Homo sapiens tubby homolog |
| | | | (mouse) (TUB), transcript variant 2, |
| | | | mRNA. |
| 2.2759655 | down | PHF17 | Homo sapiens PHD finger protein 17 |
| | | | (PHF17), transcript variant S, |
| | | | mRNA. |
| 2.2701757 | down | TAGLN3 | Homo sapiens transgelin 3 |
| | | | (TAGLN3), transcript variant 3, |
| | | | mRNA. |
| 2.2348192 | down | SYTL4 | Homo sapiens synaptotagmin-like 4 |
| | | | (granuphilin-a) (SYTL4), mRNA. |
| 2.230719 | down | ARHGDIG | Homo sapiens Rho GDP dissociation |
| | | | inhibitor (GDI) gamma |
| | | | (ARHGDIG), mRNA. |
| 2.218432 | down | ABR | Homo sapiens active BCR-related |
| | | | gene (ABR), transcript variant 2, |
| | | | mRNA. |
| 2.200697 | down | SNORA18 | Homo sapiens small nucleolar RNA, |
| | | | H/ACA box 18 (SNORA18), small |
| | | | nucleolar RNA. |
| 2.1834235 | down | H2AFY2 | Homo sapiens H2A histone family, |
| | | | member Y2 (H2AFY2), mRNA. |
| 2.1802266 | down | ST6GAL1 | Homo sapiens ST6 beta- |
| | | | galactosamide alpha-2,6- |
| | | | sialyltranferase 1 (ST6GAL1), |
| | | | transcript variant 2, mRNA. |
| 2.1800709 | down | DUSP8 | Homo sapiens dual specificity |
| | | | phosphatase 8 (DUSP8), mRNA. |
| 2.1764905 | down | TFAP2B | Homo sapiens transcription factor |
| | | | AP-2 beta (activating enhancer |
| | | | binding protein 2 beta) (TFAP2B), |
| | | | mRNA. |
| 2.1661515 | down | RCN1 | Homo sapiens reticulocalbin 1, EF- |
| | | | hand calcium binding domain |
| | | | (RCN1), mRNA. |
| 2.1537797 | down | ZNF536 | Homo sapiens zinc finger protein |
| | | | 536 (ZNF536), mRNA. |
| 2.147822 | down | F12 | Homo sapiens coagulation factor XII |
| | | | (Hageman factor) (F12), mRNA. |
| 2.1474955 | down | SCRG1 | Homo sapiens scrapie responsive |
| | | | protein 1 (SCRG1), mRNA. |
| 2.1445231 | down | LRRTM2 | Homo sapiens leucine rich repeat |
| | | | transmembrane neuronal 2 |
| | | | (LRRTM2), mRNA. |
| 2.141449 | down | GRIN1 | Homo sapiens glutamate receptor, |
| | | | ionotropic, N-methyl D-aspartate 1 |
| | | | (GRIN1), transcript variant NR1-2, |
| | | | mRNA. |
| 2.1393347 | down | SEZ6L2 | Homo sapiens seizure related 6 |
| | | | homolog (mouse)-like 2 (SEZ6L2), |
| | | | transcript variant 2, mRNA. |
| 2.1239994 | down | GRM8 | Homo sapiens glutamate receptor, |
| | | | metabotropic 8 (GRM8), mRNA. |
| 2.1140378 | down | CENTA1 | Homo sapiens centaurin, alpha 1 |
| | | | (CENTA1), mRNA. |
| 2.1079326 | down | HDGF | Homo sapiens hepatoma-derived |
| | | | growth factor (high-mobility group |
| | | | protein 1-like) (HDGF), mRNA. |
| 2.1008873 | down | JAM2 | Homo sapiens junctional adhesion |
| | | | molecule 2 (JAM2), mRNA. |
| 2.0997539 | down | DDR2 | Homo sapiens discoidin domain |
| | | | receptor tyrosine kinase 2 (DDR2), |
| | | | transcript variant 2, mRNA. |
| 2.0729117 | down | MYT1 | Homo sapiens myelin transcription |
| | | | factor 1 (MYT1), mRNA. |
| 2.0711784 | down | PCGF2 | Homo sapiens polycomb group ring |
| | | | finger 2 (PCGF2), mRNA. |
| 2.0665834 | down | | AGENCOURT_15463101 Human |
| | | | Anterior Horn Homo sapiens cDNA |
| | | | clone IMAGE: 30516556 5, mRNA |
| | | | sequence |
| 2.0662923 | down | CNTNAP1 | Homo sapiens contactin associated |
| | | | protein 1 (CNTNAP1), mRNA. |
| 2.0568583 | down | EML5 | Homo sapiens echinoderm |
| | | | microtubule associated protein like 5 |
| | | | (EML5), mRNA. |
| 2.0407145 | down | C1orf43 | Homo sapiens chromosome 1 open |
| | | | reading frame 43 (C1orf43), |
| | | | transcript variant 1, mRNA. |
| 2.0184758 | down | BRSK1 | Homo sapiens BR serine/threonine |
| | | | kinase 1 (BRSK1), mRNA. |
| 2.0163672 | down | N4BP2L1 | Homo sapiens NEDD4 binding |
| | | | protein 2-like 1 (N4BP2L1), |
| | | | transcript variant 2, mRNA. |
| 2.0149431 | down | TCEAL7 | Homo sapiens transcription |
| | | | elongation factor A (SII)-like 7 |
| | | | (TCEAL7), mRNA. |
| 2.0054402 | down | TAGLN3 | Homo sapiens transgelin 3 |
| | | | (TAGLN3), transcript variant 3, |
| | | | mRNA. |
| 2.0032253 | down | NME4 | Homo sapiens non-metastatic cells 4, |
| | | | protein expressed in (NME4), |
| | | | nuclear gene encoding mitochondrial |
| | | | protein, mRNA. |

TABLE 8

| FC (abs) | Regulation | | |
|---|---|---|---|
| (Moderated | (Moderated | | |
| T-Test | T-Test | | |
| [ATRA + | [ATRA + | | |
| Compound B] | Compound B] | | |
| Vs [DMSO] | Vs [DMSO] | | |
| P <= 0.05 | P <= 0.05 | Gene | |
| FC >= 2.0) | FC >= 2.0) | Symbol | Definition |
| 74.06221 | up | CYP26B1 | Homo sapiens cytochrome P450, |
| | | | family 26, subfamily B, polypeptide 1 |
| | | | (CYP26B1), mRNA. |
| 11.48934 | up | RET | Homo sapiens ret proto-oncogene |
| | | | (RET), transcript variant 4, mRNA. |
| 11.17362 | up | RET | Homo sapiens ret proto-oncogene |
| | | | (RET), transcript variant 2, mRNA. |
| 9.271276 | up | CRABP2 | Homo sapiens cellular retinoic acid |
| | | | binding protein 2 (CRABP2), mRNA. |
| 6.779234 | up | CYP26A1 | Homo sapiens cytochrome P450, |
| | | | family 26, subfamily A, polypeptide 1 |
| | | | (CYP26A1), transcript variant 2, |
| | | | mRNA. |
| 6.748227 | up | ATP7A | Homo sapiens ATPase, Cu++ |
| | | | transporting, alpha polypeptide |
| | | | (Menkes syndrome) (ATP7A), |
| | | | mRNA. |
| 6.434992 | up | TSPAN1 | Homo sapiens tetraspanin 1 |
| | | | (TSPAN1), mRNA. |
| 5.608586 | up | NFKBIZ | Homo sapiens nuclear factor of kappa |
| | | | light polypeptide gene enhancer in B- |
| | | | cells inhibitor, zeta (NFKBIZ), |
| | | | transcript variant 2, mRNA. |
| 5.604396 | up | DHRS3 | Homo sapiens |
| | | | dehydrogenase/reductase (SDR |
| | | | family) member 3 (DHRS3), mRNA. |
| 5.54892 | up | RARB | Homo sapiens retinoic acid receptor, |
| | | | beta (RARB), transcript variant 1, |
| | | | mRNA. |
| 5.007533 | up | PLAT | Homo sapiens plasminogen activator, |
| | | | tissue (PLAT), transcript variant 1, |
| | | | mRNA. |
| 4.24417 | up | VGF | Homo sapiens VGF nerve growth |
| | | | factor inducible (VGF), mRNA. |
| 4.239555 | up | PTGER2 | Homo sapiens prostaglandin E |
| | | | receptor 2 (subtype EP2), 53 kDa |
| | | | (PTGER2), mRNA. |
| 4.087792 | up | PCDH18 | Homo sapiens protocadherin 18 |
| | | | (PCDH18), mRNA. |
| 3.82108 | up | ENPP2 | Homo sapiens ectonucleotide |
| | | | pyrophosphatase/phosphodiesterase 2 |
| | | | (ENPP2), transcript variant 2, mRNA. |
| 3.81664 | up | NAV2 | Homo sapiens neuron navigator 2 |
| | | | (NAV2), transcript variant 2, mRNA. |
| 3.78091 | up | RARB | Homo sapiens retinoic acid receptor, |
| | | | beta (RARB), transcript variant 2, |
| | | | mRNA. |
| 3.767821 | up | PLS3 | Homo sapiens plastin 3 (T isoform) |
| | | | (PLS3), mRNA. |
| 3.743108 | up | CYP1B1 | Homo sapiens cytochrome P450, |
| | | | family 1, subfamily B, polypeptide 1 |
| | | | (CYP1B1), mRNA. |
| 3.659243 | up | LOC387763 | PREDICTED: Homo sapiens |
| | | | hypothetical LOC387763 |
| | | | (LOC387763), mRNA. |
| 3.580283 | up | PCDH18 | Homo sapiens protocadherin 18 |
| | | | (PCDH18), mRNA. |
| 3.53702 | up | PDZRN3 | PREDICTED: Homo sapiens PDZ |
| | | | domain containing RING finger 3 |
| | | | (PDZRN3), mRNA. |
| 3.466291 | up | ENPP2 | Homo sapiens ectonucleotide |
| | | | pyrophosphatase/phosphodiesterase 2 |
| | | | (ENPP2), transcript variant 2, mRNA. |
| 3.462251 | up | RET | Homo sapiens ret proto-oncogene |
| | | | (RET), transcript variant 2, mRNA. |
| 3.351228 | up | MMP11 | Homo sapiens matrix metallopeptidase |
| | | | 11 (stromelysin 3) (MMP11), mRNA. |
| 3.276531 | up | TRAF3IP2 | Homo sapiens TRAF3 interacting |
| | | | protein 2 (TRAF3IP2), transcript |
| | | | variant 2, mRNA. |
| 3.15566 | up | LOC375295 | PREDICTED: Homo sapiens |
| | | | hypothetical gene supported by |
| | | | BC013438 (LOC375295), mRNA. |
| 3.148894 | up | PRKCH | Homo sapiens protein kinase C, eta |
| | | | (PRKCH), mRNA. |
| 3.136123 | up | TMX4 | Homo sapiens thioredoxin-related |
| | | | transmembrane protein 4 (TMX4), |
| | | | mRNA. |
| 3.130793 | up | CYP26A1 | Homo sapiens cytochrome P450, |
| | | | family 26, subfamily A, polypeptide 1 |
| | | | (CYP26A1), transcript variant 2, |
| | | | mRNA. |
| 3.128071 | up | EFNB2 | Homo sapiens ephrin-B2 (EFNB2), |
| | | | mRNA. |
| 3.121308 | up | TMX4 | Homo sapiens thioredoxin-related |
| | | | transmembrane protein 4 (TMX4), |
| | | | mRNA. |
| 3.114901 | up | PDZRN3 | Homo sapiens PDZ domain containing |
| | | | ring finger 3 (PDZRN3), mRNA. |
| 3.075643 | up | FNDC5 | Homo sapiens fibronectin type III |
| | | | domain containing 5 (FNDC5), |
| | | | mRNA. |
| 3.050035 | up | NCOA3 | Homo sapiens nuclear receptor |
| | | | coactivator 3 (NCOA3), transcript |
| | | | variant 1, mRNA. |
| 2.929809 | up | THBS1 | Homo sapiens thrombospondin 1 |
| | | | (THBS1), mRNA. |
| 2.929415 | up | LOXL4 | Homo sapiens lysyl oxidase-like 4 |
| | | | (LOXL4), mRNA. |
| 2.869473 | up | CHRNA3 | Homo sapiens cholinergic receptor, |
| | | | nicotinic, alpha 3 (CHRNA3), mRNA. |
| 2.818317 | up | NAV2 | Homo sapiens neuron navigator 2 |
| | | | (NAV2), transcript variant 2, mRNA. |
| 2.737314 | up | IRF9 | Homo sapiens interferon regulatory |
| | | | factor 9 (IRF9), mRNA. |
| 2.682665 | up | REPS2 | Homo sapiens RALBP1 associated |
| | | | Eps domain containing 2 (REPS2), |
| | | | transcript variant 1, mRNA. |
| 2.665766 | up | FRMD6 | Homo sapiens FERM domain |
| | | | containing 6 (FRMD6), mRNA. |
| 2.638139 | up | NEDD4L | Homo sapiens neural precursor cell |
| | | | expressed, developmentally down- |
| | | | regulated 4-like (NEDD4L), mRNA. |
| 2.627141 | up | FOXC1 | Homo sapiens forkhead box C1 |
| | | | (FOXC1), mRNA. |
| 2.519936 | up | RARA | Homo sapiens retinoic acid receptor, |
| | | | alpha (RARA), transcript variant 1, |
| | | | mRNA. |
| 2.379357 | up | REPS2 | Homo sapiens RALBP1 associated |
| | | | Eps domain containing 2 (REPS2), |
| | | | transcript variant 1, mRNA. |
| 2.351435 | up | ABCA1 | Homo sapiens ATP-binding cassette, |
| | | | sub-family A (ABC1), member 1 |
| | | | (ABCA1), mRNA. |
| 2.325886 | up | GNG2 | Homo sapiens guanine nucleotide |
| | | | binding protein (G protein), gamma 2 |
| | | | (GNG2), mRNA. |
| 2.30321 | up | PDZRN3 | PREDICTED: Homo sapiens PDZ |
| | | | domain containing RING finger 3 |
| | | | (PDZRN3), mRNA. |
| 2.293642 | up | CHRNA3 | Homo sapiens cholinergic receptor, |
| | | | nicotinic, alpha 3 (CHRNA3), mRNA. |
| 2.217881 | up | SMOC1 | Homo sapiens SPARC related |
| | | | modular calcium binding 1 (SMOC1), |
| | | | transcript variant 1, mRNA. |
| 2.062672 | up | AKR1C3 | Homo sapiens aldo-keto reductase |
| | | | family 1, member C3 (3-alpha |
| | | | hydroxysteroid dehydrogenase, type |
| | | | II) (AKR1C3), mRNA. |
| 2.040579 | up | PRMT6 | Homo sapiens protein arginine |
| | | | methyltransferase 6 (PRMT6), |
| | | | mRNA. |
| 2.027333 | up | ALX3 | Homo sapiens aristaless-like |
| | | | homeobox 3 (ALX3), mRNA. |
| 2.025153 | up | NEDD9 | Homo sapiens neural precursor cell |
| | | | expressed, developmentally down- |
| | | | regulated 9 (NEDD9), transcript |
| | | | variant 1, mRNA. |
| 2.022927 | up | RND3 | Homo sapiens Rho family GTPase 3 |
| | | | (RND3), mRNA. |
| 2.008617 | up | C10orf33 | Homo sapiens chromosome 10 open |
| | | | reading frame 33 (C10orf33), mRNA. |
| 15.87051 | down | DLK1 | Homo sapiens delta-like 1 homolog |
| | | | (Drosophila) (DLK1), mRNA. |
| 5.487316 | down | SNHG7 | Homo sapiens small nucleolar RNA |
| | | | host gene 7 (non-protein coding) |
| | | | (SNHG7), transcript variant 1, non- |
| | | | coding RNA. |
| 4.849351 | down | MEG3 | Homo sapiens maternally expressed 3 |
| | | | (non-protein coding) (MEG3), |
| | | | transcript variant 1, non-coding RNA. |
| | | | XR_001346-XR_001372 |
| 4.700442 | down | ATP1A1 | Homo sapiens ATPase, Na+/K+ |
| | | | transporting, alpha 1 polypeptide |
| | | | (ATP1A1), transcript variant 1, |
| | | | mRNA. |
| 3.83497 | down | LOC100131866 | PREDICTED: Homo sapiens |
| | | | misc_RNA (LOC100131866), |
| | | | miscRNA. |
| 3.625778 | down | LOC728452 | PREDICTED: Homo sapiens similar |
| | | | to nuclear pore membrane protein 121 |
| | | | (LOC728452), mRNA. |
| 3.402146 | down | LOC441763 | PREDICTED: Homo sapiens |
| | | | hypothetical LOC441763 |
| | | | (LOC441763), mRNA. |
| 3.160639 | down | LOC651816 | PREDICTED: Homo sapiens similar |
| | | | to Ubiquitin-conjugating enzyme E2S |
| | | | (Ubiquitin-conjugating enzyme E2-24 |
| | | | kDa) (Ubiquitin-protein ligase) |
| | | | (Ubiquitin carrier protein) (E2-EPF5) |
| | | | (LOC651816), mRNA. |
| 3.153724 | down | CALML4 | Homo sapiens calmodulin-like 4 |
| | | | (CALML4), transcript variant 1, |
| | | | mRNA. |
| 2.967706 | down | CD320 | Homo sapiens CD320 molecule |
| | | | (CD320), mRNA. |
| 2.859124 | down | TRAP1 | Homo sapiens TNF receptor- |
| | | | associated protein 1 (TRAP1), mRNA. |
| 2.724363 | down | ST3GAL4 | Homo sapiens ST3 beta-galactoside |
| | | | alpha-2,3-sialyltransferase 4 |
| | | | (ST3GAL4), mRNA. |
| 2.705246 | down | LOC647251 | PREDICTED: Homo sapiens similar |
| | | | to maternally expressed 3 |
| | | | (LOC647251), mRNA. |
| 2.618742 | down | VIM | Homo sapiens vimentin (VIM), |
| | | | mRNA. |
| 2.558125 | down | DCN | Homo sapiens decorin (DCN), |
| | | | transcript variant C, mRNA. |
| 2.26428 | down | TRERF1 | Homo sapiens transcriptional |
| | | | regulating factor 1 (TRERF1), mRNA. |
| 2.220506 | down | SLC29A1 | Homo sapiens solute carrier family 29 |
| | | | (nucleoside transporters), member 1 |
| | | | (SLC29A1), nuclear gene encoding |
| | | | mitochondrial protein, transcript |
| | | | variant 4, mRNA. |
| 2.172436 | down | C2orf48 | Homo sapiens chromosome 2 open |
| | | | reading frame 48 (C2orf48), mRNA. |
| 2.163782 | down | INSM2 | Homo sapiens insulinoma-associated 2 |
| | | | (INSM2), mRNA. |
| 2.131659 | down | CACNA1H | Homo sapiens calcium channel, |
| | | | voltage-dependent, T type, alpha 1H |
| | | | subunit (CACNA1H), transcript |
| | | | variant 1, mRNA. |
| 2.097367 | down | ILVBL | Homo sapiens ilvB (bacterial |
| | | | acetolactate synthase)-like (ILVBL), |
| | | | mRNA. |
| 2.053179 | down | NELL1 | Homo sapiens NEL-like 1 (chicken) |
| | | | (NELL1), mRNA. |

TABLE 9

| FC (abs) | Regulation | | |
|---|---|---|---|
| (Moderated | (Moderated | | |
| T-Test | T-Test | | |
| [ATRA + | [ATRA + | | |
| Compound B] | Compound B] | | |
| Vs [DMSO] | Vs [DMSO] | | |
| P <= 0.05 | P <= 0.05 | | |
| FC >= 2.0) | FC >= 2.0) | Symbol | Definition |
| 5.310678 | up | CDKN1A | Homo sapiens cyclin-dependent |
| | | | kinase inhibitor 1A (p21, Cip1) |
| | | | (CDKN1A), transcript variant 1, |
| | | | mRNA. |
| 4.607131 | up | ACSL3 | Homo sapiens acyl-CoA synthetase |
| | | | long-chain family member 3 |
| | | | (ACSL3), transcript variant 2, mRNA. |
| 3.865349 | up | PLS3 | Homo sapiens plastin 3 (T isoform) |
| | | | (PLS3), mRNA. |
| 3.724138 | up | CRISPLD1 | Homo sapiens cysteine-rich secretory |
| | | | protein LCCL domain containing 1 |
| | | | (CRISPLD1), mRNA. |
| 3.32357 | up | CRISPLD1 | Homo sapiens cysteine-rich secretory |
| | | | protein LCCL domain containing 1 |
| | | | (CRISPLD1), mRNA. |
| 3.273512 | up | PCDH20 | Homo sapiens protocadherin 20 |
| | | | (PCDH20), mRNA. |
| 3.241042 | up | RPL26 | Homo sapiens ribosomal protein L26 |
| | | | (RPL26), mRNA. |
| 3.095257 | up | LOC729236 | PREDICTED: Homo sapiens |
| | | | misc_RNA (LOC729236), miscRNA. |
| 3.081601 | up | JARID2 | Homo sapiens jumonji, AT rich |
| | | | interactive domain 2 (JARID2), |
| | | | mRNA. |
| 2.992321 | up | RNU6-1 | Homo sapiens RNA, U6 small nuclear |
| | | | 1 (RNU6-1), small nuclear RNA. |
| 2.854101 | up | HOXD1 | Homo sapiens homeobox D1 |
| | | | (HOXD1), mRNA. |
| 2.770691 | up | ATP6AP2 | Homo sapiens ATPase, H+ |
| | | | transporting, lysosomal accessory |
| | | | protein 2 (ATP6AP2), mRNA. |
| 2.743215 | up | SPRY4 | Homo sapiens sprouty homolog 4 |
| | | | (Drosophila) (SPRY4), mRNA. |
| 2.741689 | up | REC8 | Homo sapiens REC8 homolog (yeast) |
| | | | (REC8), transcript variant 1, mRNA. |
| 2.721933 | up | FZD7 | Homo sapiens frizzled homolog 7 |
| | | | (Drosophila) (FZD7), mRNA. |
| 2.706069 | up | TMEM50B | Homo sapiens transmembrane protein |
| | | | 50B (TMEM50B), mRNA. |
| 2.705729 | up | RDH10 | Homo sapiens retinol dehydrogenase |
| | | | 10 (all-trans) (RDH10), mRNA. |
| 2.689977 | up | RN5S9 | Homo sapiens RNA, 5S ribosomal 9 |
| | | | (RN5S9), ribosomal RNA. |
| 2.68436 | up | NPTN | Homo sapiens neuroplastin (NPTN), |
| | | | transcript variant beta, mRNA. |
| 2.676171 | up | G3BP2 | Homo sapiens GTPase activating |
| | | | protein (SH3 domain) binding protein |
| | | | 2 (G3BP2), transcript variant 3, |
| | | | mRNA. |
| 2.675857 | up | ITGA1 | Homo sapiens integrin, alpha 1 |
| | | | (ITGA1), mRNA. |
| 2.665102 | up | NPTN | Homo sapiens neuroplastin (NPTN), |
| | | | transcript variant alpha, mRNA. |
| 2.656279 | up | UBLCP1 | Homo sapiens ubiquitin-like domain |
| | | | containing CTD phosphatase 1 |
| | | | (UBLCP1), mRNA. |
| 2.65175 | up | IL10RB | Homo sapiens interleukin 10 receptor, |
| | | | beta (IL10RB), mRNA. |
| 2.626543 | up | ARMET | Homo sapiens arginine-rich, mutated |
| | | | in early stage tumors (ARMET), |
| | | | mRNA. |
| 2.614088 | up | SH2B3 | Homo sapiens SH2B adaptor protein 3 |
| | | | (SH2B3), mRNA. |
| 2.60153 | up | ADD3 | Homo sapiens adducin 3 (gamma) |
| | | | (ADD3), transcript variant 3, mRNA. |
| 2.59546 | up | ACSL3 | Homo sapiens acyl-CoA synthetase |
| | | | long-chain family member 3 |
| | | | (ACSL3), transcript variant 1, mRNA. |
| 2.591278 | up | RNU6-15 | Homo sapiens RNA, U6 small nuclear |
| | | | 15 (RNU6-15), small nuclear RNA. |
| 2.589584 | up | LOC653158 | PREDICTED: Homo sapiens similar |
| | | | to hypothetical protein MGC40405, |
| | | | transcript variant 1 (LOC653158), |
| | | | mRNA. |
| 2.577032 | up | SGK1 | Homo sapiens serum/glucocorticoid |
| | | | regulated kinase 1 (SGK1), transcript |
| | | | variant 1, mRNA. |
| 2.574045 | up | ZFAND6 | Homo sapiens zinc finger, AN1-type |
| | | | domain 6 (ZFAND6), mRNA. |
| 2.567282 | up | BCHE | Homo sapiens butyrylcholinesterase |
| | | | (BCHE), mRNA. |
| 2.558458 | up | HSD17B12 | Homo sapiens hydroxysteroid (17- |
| | | | beta) dehydrogenase 12 (HSD17B12), |
| | | | mRNA. |
| 2.549309 | up | SNORA79 | Homo sapiens small nucleolar RNA, |
| | | | H/ACA box 79 (SNORA79), small |
| | | | nucleolar RNA. |
| 2.54709 | up | LIPA | Homo sapiens lipase A, lysosomal |
| | | | acid, cholesterol esterase (LIPA), |
| | | | transcript variant 2, mRNA. |
| 2.536986 | up | G3BP1 | Homo sapiens GTPase activating |
| | | | protein (SH3 domain) binding protein |
| | | | 1 (G3BP1), transcript variant 1, |
| | | | mRNA. |
| 2.509722 | up | LAMC1 | Homo sapiens laminin, gamma 1 |
| | | | (formerly LAMB2) (LAMC1), |
| | | | mRNA. |
| 2.505713 | up | CNN2 | Homo sapiens calponin 2 (CNN2), |
| | | | transcript variant 2, mRNA. |
| 2.488804 | up | ABCB1 | Homo sapiens ATP-binding cassette, |
| | | | sub-family B (MDR/TAP), member 1 |
| | | | (ABCB1), mRNA. |
| 2.481133 | up | GLCE | Homo sapiens glucuronic acid |
| | | | epimerase (GLCE), mRNA. |
| 2.477101 | up | FLOT1 | Homo sapiens flotillin 1 (FLOT1), |
| | | | mRNA. |
| 2.465013 | up | SPRED1 | Homo sapiens sprouty-related, EVH1 |
| | | | domain containing 1 (SPRED1), |
| | | | mRNA. |
| 2.463788 | up | VASN | Homo sapiens vasorin (VASN), |
| | | | mRNA. |
| 2.456818 | up | XPR1 | Homo sapiens xenotropic and |
| | | | polytropic retrovirus receptor (XPR1), |
| | | | mRNA. |
| 2.450285 | up | CYB5R4 | Homo sapiens cytochrome b5 |
| | | | reductase 4 (CYB5R4), mRNA. |
| 2.449843 | up | FAM69A | Homo sapiens family with sequence |
| | | | similarity 69, member A (FAM69A), |
| | | | mRNA. |
| 2.435929 | up | XPR1 | Homo sapiens xenotropic and |
| | | | polytropic retrovirus receptor (XPR1), |
| | | | mRNA. |
| 2.426759 | up | SC5DL | Homo sapiens sterol-C5-desaturase |
| | | | (ERG3 delta-5-desaturase homolog, |
| | | | S. cerevisiae)-like (SC5DL), transcript |
| | | | variant 1, mRNA. |
| 2.410317 | up | TMEM19 | Homo sapiens transmembrane protein |
| | | | 19 (TMEM19), mRNA. |
| 2.406851 | up | DNAJB11 | Homo sapiens DnaJ (Hsp40) homolog, |
| | | | subfamily B, member 11 (DNAJB11), |
| | | | mRNA. |
| 2.399452 | up | HSP90B1 | Homo sapiens heat shock protein |
| | | | 90 kDa beta (Grp94), member 1 |
| | | | (HSP90B1), mRNA. |
| 2.389103 | up | PAPSS1 | Homo sapiens 3′-phosphoadenosine |
| | | | 5′-phosphosulfate synthase 1 |
| | | | (PAPSS1), mRNA. |
| 2.387213 | up | | Homo sapiens primary neuroblastoma |
| | | | cDNA, clone: Nbla10111, full insert |
| | | | sequence |
| 2.376022 | up | FGFR1OP2 | Homo sapiens FGFR1 oncogene |
| | | | partner 2 (FGFR1OP2), mRNA. |
| 2.368677 | up | WDR1 | Homo sapiens WD repeat domain 1 |
| | | | (WDR1), transcript variant 2, mRNA. |
| 2.362372 | up | HSD17B12 | Homo sapiens hydroxysteroid (17- |
| | | | beta) dehydrogenase 12 (HSD17B12), |
| | | | mRNA. |
| 2.358175 | up | WDR44 | Homo sapiens WD repeat domain 44 |
| | | | (WDR44), mRNA. |
| 2.354716 | up | OSTF1 | Homo sapiens osteoclast stimulating |
| | | | factor 1 (OSTF1), mRNA. |
| 2.338338 | up | SGK1 | Homo sapiens serum/glucocorticoid |
| | | | regulated kinase 1 (SGK1), transcript |
| | | | variant 1, mRNA. |
| 2.338305 | up | S100A10 | Homo sapiens S100 calcium binding |
| | | | protein A10 (S100A10), mRNA. |
| 2.333673 | up | SIPA1 | Homo sapiens signal-induced |
| | | | proliferation-associated gene 1 |
| | | | (SIPA1), transcript variant 2, mRNA. |
| 2.317173 | up | SCGN | Homo sapiens secretagogin, EF-hand |
| | | | calcium binding protein (SCGN), |
| | | | mRNA. |
| 2.315298 | up | PLS1 | Homo sapiens plastin 1 (I isoform) |
| | | | (PLS1), mRNA. |
| 2.314937 | up | RALB | Homo sapiens v-ral simian leukemia |
| | | | viral oncogene homolog B (ras |
| | | | related; GTP binding protein) |
| | | | (RALB), mRNA. |
| 2.308636 | up | TMC6 | Homo sapiens transmembrane |
| | | | channel-like 6 (TMC6), mRNA. |
| 2.302612 | up | EXTL2 | Homo sapiens exostoses (multiple)- |
| | | | like 2 (EXTL2), transcript variant 1, |
| | | | mRNA. |
| 2.2959 | up | PNPLA8 | Homo sapiens patatin-like |
| | | | phospholipase domain containing 8 |
| | | | (PNPLA8), mRNA. |
| 2.274158 | up | YIPF1 | Homo sapiens Yip1 domain family, |
| | | | member 1 (YIPF1), mRNA. |
| 2.256347 | up | GPR177 | Homo sapiens G protein-coupled |
| | | | receptor 177 (GPR177), transcript |
| | | | variant 1, mRNA. |
| 2.251912 | up | TRAM2 | Homo sapiens translocation associated |
| | | | membrane protein 2 (TRAM2), |
| | | | mRNA. |
| 2.246115 | up | CXorf57 | Homo sapiens chromosome X open |
| | | | reading frame 57 (CXorf57), mRNA. |
| 2.239622 | up | MYCNOS | PREDICTED: Homo sapiens |
| | | | misc_RNA (MYCNOS), miscRNA. |
| 2.233039 | up | COQ10B | Homo sapiens coenzyme Q10 |
| | | | homolog B (S. cerevisiae) (COQ10B), |
| | | | mRNA. |
| 2.232365 | up | PIGM | Homo sapiens phosphatidylinositol |
| | | | glycan anchor biosynthesis, class M |
| | | | (PIGM), mRNA. |
| 2.231697 | up | ELMOD1 | Homo sapiens ELMO/CED-12 |
| | | | domain containing 1 (ELMOD1), |
| | | | mRNA. |
| 2.228658 | up | DNAJB6 | Homo sapiens DnaJ (Hsp40) homolog, |
| | | | subfamily B, member 6 (DNAJB6), |
| | | | transcript variant 1, mRNA. |
| 2.225392 | up | LOC653156 | PREDICTED: Homo sapiens similar |
| | | | to hCG1782414 (LOC653156), |
| | | | mRNA. |
| 2.224361 | up | REC8 | Homo sapiens REC8 homolog (yeast) |
| | | | (REC8), transcript variant 1, mRNA. |
| 2.223486 | up | TMBIM4 | Homo sapiens transmembrane BAX |
| | | | inhibitor motif containing 4 |
| | | | (TMBIM4), mRNA. |
| 2.22101 | up | TJP1 | Homo sapiens tight junction protein 1 |
| | | | (zona occludens 1) (TJP1), transcript |
| | | | variant 2, mRNA. |
| 2.212364 | up | USP8 | Homo sapiens ubiquitin specific |
| | | | peptidase 8 (USP8), mRNA. |
| 2.208929 | up | OSBPL3 | Homo sapiens oxysterol binding |
| | | | protein-like 3 (OSBPL3), transcript |
| | | | variant 4, mRNA. |
| 2.203524 | up | CPVL | Homo sapiens carboxypeptidase, |
| | | | vitellogenic-like (CPVL), transcript |
| | | | variant 2, mRNA. |
| 2.197295 | up | DUSP5 | Homo sapiens dual specificity |
| | | | phosphatase 5 (DUSP5), mRNA. |
| 2.194288 | up | CADM1 | Homo sapiens cell adhesion molecule |
| | | | 1 (CADM1), transcript variant 1, |
| | | | mRNA. |
| 2.193162 | up | SEC24D | Homo sapiens SEC24 related gene |
| | | | family, member D (S. cerevisiae) |
| | | | (SEC24D), mRNA. |
| 2.189749 | up | MY ADM | Homo sapiens myeloid-associated |
| | | | differentiation marker (MYADM), |
| | | | transcript variant 4, mRNA. |
| 2.185441 | up | LOC285359 | Homo sapiens phosducin-like 3 |
| | | | pseudogene (LOC285359) on |
| | | | chromosome 3. |
| 2.177089 | up | MYL12A | Homo sapiens myosin, light chain |
| | | | 12A, regulatory, non-sarcomeric |
| | | | (MYL12A), mRNA. |
| 2.175736 | up | C3orf59 | Homo sapiens chromosome 3 open |
| | | | reading frame 59 (C3orf59), mRNA. |
| 2.169516 | up | BCL6 | Homo sapiens B-cell CLL/lymphoma |
| | | | 6 (zinc finger protein 51) (BCL6), |
| | | | transcript variant 1, mRNA. |
| 2.168062 | up | EPB41L5 | Homo sapiens erythrocyte membrane |
| | | | protein band 4.1 like 5 (EPB41L5), |
| | | | mRNA. |
| 2.167211 | up | CXorf45 | Homo sapiens chromosome X open |
| | | | reading frame 45 (CXorf45), transcript |
| | | | variant 1, mRNA. |
| 2.161228 | up | ZSWIM6 | PREDICTED: Homo sapiens zinc |
| | | | finger, SWIM-type containing 6 |
| | | | (ZSWIM6), mRNA. |
| 2.158413 | up | DCBLD2 | Homo sapiens discoidin, CUB and |
| | | | LCCL domain containing 2 |
| | | | (DCBLD2), mRNA. |
| 2.157928 | up | LAMP2 | Homo sapiens lysosomal-associated |
| | | | membrane protein 2 (LAMP2), |
| | | | transcript variant LAMP2B, mRNA. |
| 2.157778 | up | HLA-B | Homo sapiens major |
| | | | histocompatibility complex, class I, B |
| | | | (HLA-B), mRNA. |
| 2.155381 | up | | PREDICTED: Homo sapiens |
| | | | hypothetical LOC389089 |
| | | | (LOC389089), mRNA |
| 2.154519 | up | LOC401076 | PREDICTED: Homo sapiens |
| | | | misc_RNA (LOC401076), miscRNA. |
| 2.153577 | up | TXNDC9 | Homo sapiens thioredoxin domain |
| | | | containing 9 (TXNDC9), mRNA. |
| 2.149624 | up | PCDH17 | Homo sapiens protocadherin 17 |
| | | | (PCDH17), mRNA. |
| 2.148683 | up | | Homo sapiens cDNA FLJ26539 fis, |
| | | | clone KDN09310 |
| 2.147121 | up | YIPF1 | Homo sapiens Yip1 domain family, |
| | | | member 1 (YIPF1), mRNA. |
| 2.135235 | up | LOC729646 | PREDICTED: Homo sapiens |
| | | | misc_RNA (LOC729646), miscRNA. |
| 2.133704 | up | PTGR1 | Homo sapiens prostaglandin reductase |
| | | | 1 (PTGR1), mRNA. |
| 2.133145 | up | IGF2R | Homo sapiens insulin-like growth |
| | | | factor 2 receptor (IGF2R), mRNA. |
| 2.133046 | up | EPB41L5 | Homo sapiens erythrocyte membrane |
| | | | protein band 4.1 like 5 (EPB41L5), |
| | | | mRNA. |
| 2.132543 | up | LOC100129685 | PREDICTED: Homo sapiens |
| | | | hypothetical protein LOC100129685 |
| | | | (LOC100129685), mRNA. |
| 2.130848 | up | PAQR8 | Homo sapiens progestin and adipoQ |
| | | | receptor family member VIII |
| | | | (PAQR8), mRNA. |
| 2.124304 | up | RPGR | Homo sapiens retinitis pigmentosa |
| | | | GTPase regulator (RPGR), transcript |
| | | | variant B, mRNA. |
| 2.122092 | up | FBLN2 | Homo sapiens fibulin 2 (FBLN2), |
| | | | transcript variant 2, mRNA. |
| 2.121826 | up | GCA | Homo sapiens grancalcin, EF-hand |
| | | | calcium binding protein (GCA), |
| | | | mRNA. |
| 2.120949 | up | GPR126 | Homo sapiens G protein-coupled |
| | | | receptor 126 (GPR126), transcript |
| | | | variant a2, mRNA. |
| 2.118829 | up | | Homo sapiens mRNA; cDNA |
| | | | DKFZp564C152 (from clone |
| | | | DKFZp564C152) |
| 2.118362 | up | PI15 | Homo sapiens peptidase inhibitor 15 |
| | | | (PI15), mRNA. |
| 2.116085 | up | GNS | Homo sapiens glucosamine (N- |
| | | | acetyl)-6-sulfatase (Sanfilippo disease |
| | | | IIID) (GNS), mRNA. |
| 2.110086 | up | ALG13 | Homo sapiens asparagine-linked |
| | | | glycosylation 13 homolog |
| | | | (S. cerevisiae) (ALG13), mRNA. |
| 2.109918 | up | TP53INP1 | Homo sapiens tumor protein p53 |
| | | | inducible nuclear protein 1 |
| | | | (TP53INP1), mRNA. |
| 2.109562 | up | NPPA | Homo sapiens natriuretic peptide |
| | | | precursor A (NPPA), mRNA. |
| 2.108881 | up | USP38 | Homo sapiens ubiquitin specific |
| | | | peptidase 38 (USP38), mRNA. |
| 2.108092 | up | PSMA4 | Homo sapiens proteasome (prosome, |
| | | | macropain) subunit, alpha type, 4 |
| | | | (PSMA4), mRNA. |
| 2.10809 | up | C5orf32 | Homo sapiens chromosome 5 open |
| | | | reading frame 32 (C5orf32), mRNA. |
| 2.095516 | up | PRKCA | Homo sapiens protein kinase C, alpha |
| | | | (PRKCA), mRNA. |
| 2.094473 | up | | Homo sapiens cDNA clone |
| | | | IMAGE: 5268658 |
| 2.090239 | up | SEC22B | Homo sapiens SEC22 vesicle |
| | | | trafficking protein homolog B |
| | | | (S. cerevisiae) (SEC22B), mRNA. |
| 2.084241 | up | DNAJC10 | Homo sapiens DnaJ (Hsp40) homolog, |
| | | | subfamily C, member 10 (DNAJC10), |
| | | | mRNA. |
| 2.083668 | up | UTP14C | Homo sapiens UTP14, U3 small |
| | | | nucleolar ribonucleoprotein, homolog |
| | | | C (yeast) (UTP14C), mRNA. |
| 2.082504 | up | TULP4 | Homo sapiens tubby like protein 4 |
| | | | (TULP4), transcript variant 2, mRNA. |
| 2.080901 | up | HIF1A | Homo sapiens hypoxia-inducible |
| | | | factor 1, alpha subunit (basic helix- |
| | | | loop-helix transcription factor) |
| | | | (HIF1A), transcript variant 2, mRNA. |
| 2.079186 | up | DYNC1I1 | Homo sapiens dynein, cytoplasmic 1, |
| | | | intermediate chain 1 (DYNC1I1), |
| | | | mRNA. |
| 2.077066 | up | ANKRD57 | Homo sapiens ankyrin repeat domain |
| | | | 57 (ANKRD57), mRNA. |
| 2.073996 | up | PON2 | Homo sapiens paraoxonase 2 (PON2), |
| | | | transcript variant 1, mRNA. |
| 2.071366 | up | BMPR2 | Homo sapiens bone morphogenetic |
| | | | protein receptor, type II |
| | | | (serine/threonine kinase) (BMPR2), |
| | | | mRNA. |
| 2.069789 | up | SLC4A8 | Homo sapiens solute carrier family 4, |
| | | | sodium bicarbonate cotransporter, |
| | | | member 8 (SLC4A8), transcript |
| | | | variant 2, mRNA. |
| 2.067038 | up | ATP2B1 | Homo sapiens ATPase, Ca++ |
| | | | transporting, plasma membrane 1 |
| | | | (ATP2B1), transcript variant 1, |
| | | | mRNA. |
| 2.066323 | up | DAD1 | Homo sapiens defender against cell |
| | | | death 1 (DAD1), mRNA. |
| 2.063078 | up | RAB3IP | Homo sapiens RAB3A interacting |
| | | | protein (rabin3) (RAB3IP), transcript |
| | | | variant beta 1, mRNA. |
| 2.062607 | up | RPPH1 | Homo sapiens ribonuclease P RNA |
| | | | component H1 (RPPH1), RNase P |
| | | | RNA. |
| 2.059798 | up | PRG2 | Homo sapiens proteoglycan 2, bone |
| | | | marrow (natural killer cell activator, |
| | | | eosinophil granule major basic |
| | | | protein) (PRG2), mRNA. |
| 2.056469 | up | PRKAR1A | Homo sapiens protein kinase, cAMP- |
| | | | dependent, regulatory, type I, alpha |
| | | | (tissue specific extinguisher 1) |
| | | | (PRKAR1A), transcript variant 1, |
| | | | mRNA. |
| 2.056032 | up | ZMYM1 | Homo sapiens zinc finger, MYM-type |
| | | | 1 (ZMYM1), mRNA. |
| 2.050947 | up | CLINT 1 | Homo sapiens clathrin interactor 1 |
| | | | (CLINT1), mRNA. |
| 2.050481 | up | TMCO1 | Homo sapiens transmembrane and |
| | | | coiled-coil domains 1 (TMCO1), |
| | | | mRNA. |
| 2.048413 | up | PDGFD | Homo sapiens platelet derived growth |
| | | | factor D (PDGFD), transcript variant |
| | | | 1, mRNA. |
| 2.047608 | up | USP9X | Homo sapiens ubiquitin specific |
| | | | peptidase 9, X-linked (USP9X), |
| | | | transcript variant 4, mRNA. |
| 2.046355 | up | AADACL4 | Homo sapiens arylacetamide |
| | | | deacetylase-like 4 (AADACL4), |
| | | | mRNA. |
| 2.046026 | up | BCL2L12 | Homo sapiens BCL2-like 12 (proline |
| | | | rich) (BCL2L12), transcript variant 3, |
| | | | mRNA. |
| 2.045556 | up | ALPL | Homo sapiens alkaline phosphatase, |
| | | | liver/bone/kidney (ALPL), transcript |
| | | | variant 1, mRNA. |
| 2.040306 | up | LOC653079 | PREDICTED: Homo sapiens |
| | | | misc_RNA (LOC653079), miscRNA. |
| 2.037734 | up | CCDC128 | Homo sapiens coiled-coil domain |
| | | | containing 128 (CCDC128), mRNA. |
| 2.034251 | up | HDAC1 | Homo sapiens histone deacetylase 1 |
| | | | (HDAC1), mRNA. |
| 2.032832 | up | HLA-E | Homo sapiens major |
| | | | histocompatibility complex, class I, E |
| | | | (HLA-E), mRNA. |
| 2.032317 | up | INTS6 | Homo sapiens integrator complex |
| | | | subunit 6 (INTS6), transcript variant |
| | | | 2, mRNA. |
| 2.032247 | up | TMEM166 | Homo sapiens transmembrane protein |
| | | | 166 (TMEM166), mRNA. |
| 2.031952 | up | NDFIP2 | Homo sapiens Nedd4 family |
| | | | interacting protein 2 (NDFIP2), |
| | | | mRNA. |
| 2.031459 | up | EDEM3 | Homo sapiens ER degradation |
| | | | enhancer, mannosidase alpha-like 3 |
| | | | (EDEM3), mRNA. |
| 2.030964 | up | FER1L4 | Homo sapiens fer-1-like 4 (C. elegans) |
| | | | (FER1L4) on chromosome 20. |
| 2.029642 | up | CHUK | Homo sapiens conserved helix-loop- |
| | | | helix ubiquitous kinase (CHUK), |
| | | | mRNA. |
| 2.028674 | up | C10orf75 | PREDICTED: Homo sapiens |
| | | | misc_RNA (C10orf75), miscRNA. |
| 2.025385 | up | LOC389342 | PREDICTED: Homo sapiens similar |
| | | | to QM protein, transcript variant 11 |
| | | | (LOC389342), mRNA. |
| 2.025148 | up | RNASEL | Homo sapiens ribonuclease L (2′,5- |
| | | | oligoisoadenylate synthetase- |
| | | | dependent) (RNASEL), mRNA. |
| 2.024918 | up | LOC100131205 | PREDICTED: Homo sapiens |
| | | | hypothetical protein LOC100131205 |
| | | | (LOC100131205), mRNA. |
| 2.021271 | up | TMEM205 | Homo sapiens transmembrane protein |
| | | | 205 (TMEM205), mRNA. |
| 2.019676 | up | RRBP1 | Homo sapiens ribosome binding |
| | | | protein 1 homolog 180 kDa (dog) |
| | | | (RRBP1), transcript variant 1, mRNA. |
| 2.017824 | up | ALCAM | Homo sapiens activated leukocyte cell |
| | | | adhesion molecule (ALCAM), |
| | | | mRNA. |
| 2.014654 | up | ATG4C | Homo sapiens ATG4 autophagy |
| | | | related 4 homolog C (S. cerevisiae) |
| | | | (ATG4C), transcript variant 7, mRNA. |
| 2.014039 | up | MEGF9 | Homo sapiens multiple EGF-like- |
| | | | domains 9 (MEGF9), mRNA. |
| 2.013871 | up | C1orf97 | Homo sapiens chromosome 1 open |
| | | | reading frame 97 (C1orf97), mRNA. |
| 2.013175 | up | STRADB | Homo sapiens STE20-related kinase |
| | | | adaptor beta (STRADB), mRNA. |
| 2.01128 | up | SREBF1 | Homo sapiens sterol regulatory |
| | | | element binding transcription factor 1 |
| | | | (SREBF1), transcript variant 1, |
| | | | mRNA. |
| 2.010292 | up | SUOX | Homo sapiens sulfite oxidase |
| | | | (SUOX), nuclear gene encoding |
| | | | mitochondrial protein, transcript |
| | | | variant 1, mRNA. |
| 2.007771 | up | RAB8B | Homo sapiens RAB8B, member RAS |
| | | | oncogene family (RAB8B), mRNA. |
| 2.006673 | up | SPRY1 | Homo sapiens sprouty homolog 1, |
| | | | antagonist of FGF signaling |
| | | | (Drosophila) (SPRY1), transcript |
| | | | variant 1, mRNA. |
| 2.006646 | up | ARL6IP1 | Homo sapiens ADP-ribosylation |
| | | | factor-like 6 interacting protein 1 |
| | | | (ARL6IP1), mRNA. |
| 2.005585 | up | C12orf34 | Homo sapiens chromosome 12 open |
| | | | reading frame 34 (C12orf34), mRNA. |
| 2.003665 | up | RPAP3 | Homo sapiens RNA polymerase II |
| | | | associated protein 3 (RPAP3), mRNA. |
| 2.00341 | up | LOC728782 | PREDICTED: Homo sapiens similar |
| | | | to ribosomal protein L21 |
| | | | (LOC728782), mRNA. |
| 2.002626 | up | PLEKHA6 | Homo sapiens pleckstrin homology |
| | | | domain containing, family A member |
| | | | 6 (PLEKHA6), mRNA. |
| 2.000571 | up | KLF10 | Homo sapiens Kruppel-like factor 10 |
| | | | (KLF10), transcript variant 2, mRNA. |
| 8.136414 | down | LOC648210 | PREDICTED: Homo sapiens similar |
| | | | to Heterogeneous nuclear |
| | | | ribonucleoprotein A1 (Helix- |
| | | | destabilizing protein) (Single-strand |
| | | | RNA-binding protein) (hnRNP core |
| | | | protein A1) (HDP) (LOC648210), |
| | | | mRNA. |
| 6.259928 | down | TUBA1A | Homo sapiens tubulin, alpha 1a |
| | | | (TUBA1A), mRNA. |
| 6.153608 | down | ACTG1 | Homo sapiens actin, gamma 1 |
| | | | (ACTG1), mRNA. |
| 5.798153 | down | LOC100008588 | Homo sapiens 18S ribosomal RNA |
| | | | (LOC100008588), non-coding RNA. |
| 5.754853 | down | LOC100133565 | PREDICTED: Homo sapiens similar |
| | | | to hCG23738 (LOC100133565), |
| | | | mRNA. |
| 5.722868 | down | TUBB | Homo sapiens tubulin, beta (TUBB), |
| | | | mRNA. |
| 5.397586 | down | LOC92755 | PREDICTED: Homo sapiens |
| | | | misc_RNA (LOC92755), miscRNA. |
| 5.187223 | down | LOC100133372 | PREDICTED: Homo sapiens |
| | | | misc_RNA (LOC100133372), |
| | | | miscRNA. |
| 4.373462 | down | TUBA1C | Homo sapiens tubulin, alpha 1c |
| | | | (TUBA1C), mRNA. |
| 4.353879 | down | ACTB | Homo sapiens actin, beta (ACTB), |
| | | | mRNA. |
| 4.222068 | down | RTN1 | Homo sapiens reticulon 1 (RTN1), |
| | | | transcript variant 3, mRNA. |
| 4.119943 | down | LOC642817 | PREDICTED: Homo sapiens |
| | | | hypothetical LOC642817 |
| | | | (LOC642817), mRNA. |
| 4.083703 | down | FLJ39632 | PREDICTED: Homo sapiens |
| | | | misc_RNA (FLJ39632), miscRNA. |
| 4.034211 | down | LOC91561 | PREDICTED: Homo sapiens similar |
| | | | to ribosomal protein S2, transcript |
| | | | variant 3 (LOC91561), mRNA. |
| 3.980733 | down | LOC645691 | PREDICTED: Homo sapiens similar |
| | | | to heterogeneous nuclear |
| | | | ribonucleoprotein A1 (LOC645691), |
| | | | mRNA. |
| 3.887888 | down | LOC100131609 | PREDICTED: Homo sapiens |
| | | | misc_RNA (LOC100131609), |
| | | | miscRNA. |
| 3.809681 | down | PHOX2B | Homo sapiens paired-like homeobox |
| | | | 2b (PHOX2B), mRNA. |
| 3.807991 | down | LOC388654 | PREDICTED: Homo sapiens similar |
| | | | to laminin receptor 1 (ribosomal |
| | | | protein SA) (LOC388654), mRNA. |
| 3.683417 | down | RPLP0 | Homo sapiens ribosomal protein, |
| | | | large, P0 (RPLP0), transcript variant |
| | | | 1, mRNA. |
| 3.642376 | down | PHOX2B | Homo sapiens paired-like homeobox |
| | | | 2b (PHOX2B), mRNA. |
| 3.548049 | down | IRF2BP2 | Homo sapiens interferon regulatory |
| | | | factor 2 binding protein 2 (IRF2BP2), |
| | | | transcript variant 1, mRNA. |
| 3.533466 | down | TMEM132A | Homo sapiens transmembrane protein |
| | | | 132A (TMEM132A), transcript |
| | | | variant 2, mRNA. |
| 3.521768 | down | CCT7 | Homo sapiens chaperonin containing |
| | | | TCP1, subunit 7 (eta) (CCT7), |
| | | | transcript variant 1, mRNA. |
| 3.502324 | down | SIX3 | Homo sapiens sine oculis homeobox |
| | | | homolog 3 (Drosophila) (SIX3), |
| | | | mRNA. |
| 3.466076 | down | LOC645436 | PREDICTED: Homo sapiens similar |
| | | | to Heterogeneous nuclear |
| | | | ribonucleoprotein A1 (Helix- |
| | | | destabilizing protein) (Single-strand |
| | | | binding protein) (hnRNP core protein |
| | | | A1) (HDP-1) (Topoisomerase- |
| | | | inhibitor suppressed) (LOC645436), |
| | | | mRNA. |
| 3.374453 | down | LOC648210 | PREDICTED: Homo sapiens similar |
| | | | to Heterogeneous nuclear |
| | | | ribonucleoprotein A1 (Helix- |
| | | | destabilizing protein) (Single-strand |
| | | | RNA-binding protein) (hnRNP core |
| | | | protein A1) (HDP) (LOC648210), |
| | | | mRNA. |
| 3.339326 | down | HMGA1 | Homo sapiens high mobility group |
| | | | AT-hook 1 (HMGA1), transcript |
| | | | variant 1, mRNA. |
| 3.33026 | down | LOC148430 | PREDICTED: Homo sapiens |
| | | | misc_RNA (LOC148430), miscRNA. |
| 3.297835 | down | RPS2 | Homo sapiens ribosomal protein S2 |
| | | | (RPS2), mRNA. |
| 3.2824 | down | LOC645385 | PREDICTED: Homo sapiens similar |
| | | | to heterogeneous nuclear |
| | | | ribonucleoprotein A1 (LOC645385), |
| | | | mRNA. |
| 3.280514 | down | ALDOA | Homo sapiens aldolase A, fructose- |
| | | | bisphosphate (ALDOA), transcript |
| | | | variant 2, mRNA. |
| 3.279588 | down | LOC728698 | PREDICTED: Homo sapiens |
| | | | misc_RNA (LOC728698), miscRNA. |
| 3.266189 | down | EEF1G | Homo sapiens eukaryotic translation |
| | | | elongation factor 1 gamma (EEF1G), |
| | | | mRNA. XM_935976 XM_935977 |
| | | | XM_935978 XM_935979 |
| 3.255224 | down | LOC728643 | Homo sapiens heterogeneous nuclear |
| | | | ribonucleoprotein A1 pseudogene |
| | | | (LOC728643), non-coding RNA. |
| 3.249131 | down | RPLP0 | Homo sapiens ribosomal protein, |
| | | | large, P0 (RPLP0), transcript variant |
| | | | 2, mRNA. |
| 3.246931 | down | SORBS2 | Homo sapiens sorbin and SH3 domain |
| | | | containing 2 (SORBS2), transcript |
| | | | variant 1, mRNA. |
| 3.222426 | down | MYCN | Homo sapiens v-myc |
| | | | myelocytomatosis viral related |
| | | | oncogene, neuroblastoma derived |
| | | | (avian) (MYCN), mRNA. |
| 3.216845 | down | GUSBL1 | Homo sapiens glucuronidase, beta-like |
| | | | 1 (GUSBL1), non-coding RNA. |
| 3.189626 | down | SORBS2 | Homo sapiens sorbin and SH3 domain |
| | | | containing 2 (SORBS2), transcript |
| | | | variant 1, mRNA. |
| 3.171891 | down | RPS9 | Homo sapiens ribosomal protein S9 |
| | | | (RPS9), mRNA. |
| 3.160895 | down | LOC729926 | PREDICTED: Homo sapiens |
| | | | misc_RNA (LOC729926), miscRNA. |
| 3.14376 | down | C1orf43 | Homo sapiens chromosome 1 open |
| | | | reading frame 43 (C1orf43), transcript |
| | | | variant 1, mRNA. |
| 3.11659 | down | LOC100008589 | Homo sapiens 28S ribosomal RNA |
| | | | (LOC100008589), non-coding RNA. |
| 3.103844 | down | GTF2IP1 | Homo sapiens general transcription |
| | | | factor II, i, pseudogene 1 (GTF2IP1) |
| | | | on chromosome 7. |
| 3.101386 | down | ATP1A1 | Homo sapiens ATPase, Na+/K+ |
| | | | transporting, alpha 1 polypeptide |
| | | | (ATP1A1), transcript variant 1, |
| | | | mRNA. |
| 3.069661 | down | LOC646294 | PREDICTED: Homo sapiens |
| | | | misc_RNA (LOC646294), miscRNA. |
| 3.067323 | down | LOC391075 | PREDICTED: Homo sapiens |
| | | | misc_RNA (LOC391075), miscRNA. |
| 3.065105 | down | LOC402112 | PREDICTED: Homo sapiens |
| | | | misc_RNA (LOC402112), miscRNA. |
| 3.040335 | down | ALDOA | Homo sapiens aldolase A, fructose- |
| | | | bisphosphate (ALDOA), transcript |
| | | | variant 3, mRNA. |
| 3.033488 | down | LOC728565 | PREDICTED: Homo sapiens similar |
| | | | to Beta-glucuronidase precursor |
| | | | (LOC728565), mRNA. |
| 3.02633 | down | LOC646785 | PREDICTED: Homo sapiens |
| | | | misc_RNA (LOC646785), miscRNA. |
| 3.002853 | down | RPS9 | Homo sapiens ribosomal protein S9 |
| | | | (RPS9), mRNA. |
| 2.996558 | down | TPI1 | Homo sapiens triosephosphate |
| | | | isomerase 1 (TPI1), mRNA. |
| 2.988225 | down | TCP1 | Homo sapiens t-complex 1 (TCP1), |
| | | | transcript variant 1, mRNA. |
| 2.981205 | down | LOC644063 | PREDICTED: Homo sapiens similar |
| | | | to heterogeneous nuclear |
| | | | ribonucleoprotein K (LOC644063), |
| | | | mRNA. |
| 2.977413 | down | APP | Homo sapiens amyloid beta (A4) |
| | | | precursor protein (APP), transcript |
| | | | variant 3, mRNA. |
| 2.972969 | down | LOC440589 | PREDICTED: Homo sapiens similar |
| | | | to ribosomal protein S2, transcript |
| | | | variant 3 (LOC440589), mRNA. |
| 2.94257 | down | LOC284821 | PREDICTED: Homo sapiens similar |
| | | | to ribosomal protein L13a |
| | | | (LOC284821), mRNA. |
| 2.913472 | down | LOC100129553 | PREDICTED: Homo sapiens |
| | | | misc_RNA (LOC100129553), |
| | | | miscRNA. |
| 2.911284 | down | PGAM1 | Homo sapiens phosphoglycerate |
| | | | mutase 1 (brain) (PGAM1), mRNA. |
| 2.884164 | down | LOC643357 | PREDICTED: Homo sapiens similar |
| | | | to SMT3 suppressor of mif two 3 |
| | | | homolog 2 (LOC643357), mRNA. |
| 2.862033 | down | PRMT1 | Homo sapiens protein arginine |
| | | | methyltransferase 1 (PRMT1), |
| | | | transcript variant 2, mRNA. |
| 2.849308 | down | PLD6 | Homo sapiens phospholipase D |
| | | | family, member 6 (PLD6), mRNA. |
| 2.848781 | down | LOC647000 | PREDICTED: Homo sapiens similar |
| | | | to tubulin, beta 5 (LOC647000), |
| | | | mRNA. |
| 2.848405 | down | PRDX2 | Homo sapiens peroxiredoxin 2 |
| | | | (PRDX2), nuclear gene encoding |
| | | | mitochondrial protein, transcript |
| | | | variant 3, mRNA. |
| 2.834647 | down | HAND2 | Homo sapiens heart and neural crest |
| | | | derivatives expressed 2 (HAND2), |
| | | | mRNA. |
| 2.828951 | down | LOC100131609 | PREDICTED: Homo sapiens |
| | | | misc_RNA (LOC100131609), |
| | | | miscRNA. |
| 2.828043 | down | GTF2IP1 | Homo sapiens general transcription |
| | | | factor II, i, pseudogene 1 (GTF2IP1) |
| | | | on chromosome 7. |
| 2.821679 | down | MATR3 | Homo sapiens matrin 3 (MATR3), |
| | | | transcript variant 1, mRNA. |
| 2.819402 | down | ATF4 | Homo sapiens activating transcription |
| | | | factor 4 (tax-responsive enhancer |
| | | | element B67) (ATF4), transcript |
| | | | variant 2, mRNA. |
| 2.81394 | down | LOC100132528 | PREDICTED: Homo sapiens |
| | | | misc_RNA (LOC100132528), |
| | | | miscRNA. |
| 2.810629 | down | LOC347544 | PREDICTED: Homo sapiens similar |
| | | | to ribosomal protein L18a |
| | | | (LOC347544), mRNA. |
| 2.803635 | down | LOC440589 | PREDICTED: Homo sapiens similar |
| | | | to ribosomal protein S2, transcript |
| | | | variant 3 (LOC440589), mRNA. |
| 2.795735 | down | PLCXD3 | Homo sapiens phosphatidylinositol- |
| | | | specific phospholipase C, X domain |
| | | | containing 3 (PLCXD3), mRNA. |
| 2.794639 | down | LOC728658 | PREDICTED: Homo sapiens similar |
| | | | to 23 kD highly basic protein, |
| | | | transcript variant 1 (LOC728658), |
| | | | mRNA. |
| 2.784671 | down | LOC651149 | PREDICTED: Homo sapiens similar |
| | | | to 60S ribosomal protein L3 (L4) |
| | | | (LOC651149), mRNA. |
| 2.761892 | down | PRDX2 | Homo sapiens peroxiredoxin 2 |
| | | | (PRDX2), nuclear gene encoding |
| | | | mitochondrial protein, transcript |
| | | | variant 3, mRNA. |
| 2.755405 | down | SNHG7 | Homo sapiens small nucleolar RNA |
| | | | host gene 7 (non-protein coding) |
| | | | (SNHG7), transcript variant 2, non- |
| | | | coding RNA. |
| 2.755216 | down | LOC729779 | PREDICTED: Homo sapiens |
| | | | misc_RNA (LOC729779), miscRNA. |
| 2.739008 | down | NCL | Homo sapiens nucleolin (NCL), |
| | | | mRNA. |
| 2.734261 | down | LOC285053 | PREDICTED: Homo sapiens similar |
| | | | to ribosomal protein L18a, transcript |
| | | | variant 1 (LOC285053), mRNA. |
| 2.723704 | down | MTHFD2 | Homo sapiens |
| | | | methylenetetrahydrofolate |
| | | | dehydrogenase (NADP+ dependent) 2, |
| | | | methenyltetrahydrofolate |
| | | | cyclohydrolase (MTHFD2), nuclear |
| | | | gene encoding mitochondrial protein, |
| | | | transcript variant 2, mRNA. |
| 2.723382 | down | SMA4 | Homo sapiens SMA4 (SMA4), |
| | | | mRNA. |
| 2.709106 | down | LOC441775 | PREDICTED: Homo sapiens similar |
| | | | to 60S ribosomal protein L18 |
| | | | (LOC441775), mRNA. |
| 2.7038 | down | CAPRIN1 | Homo sapiens cell cycle associated |
| | | | protein 1 (CAPRIN1), transcript |
| | | | variant 1, mRNA. |
| 2.69812 | down | LOC648695 | PREDICTED: Homo sapiens similar |
| | | | to retinoblastoma binding protein 4, |
| | | | transcript variant 5 (LOC648695), |
| | | | mRNA. |
| 2.695318 | down | LOC648249 | PREDICTED: Homo sapiens similar |
| | | | to 40S ribosomal protein SA (p40) |
| | | | (34/67 kDa laminin receptor) (Colon |
| | | | carcinoma laminin-binding protein) |
| | | | (NEM/1CHD4) (Multidrug resistance- |
| | | | associated protein MGr1-Ag), |
| | | | transcript variant 3 (LOC648249), |
| | | | mRNA. |
| 2.689333 | down | HIST3H2A | Homo sapiens histone cluster 3, H2a |
| | | | (HIST3H2A), mRNA. |
| 2.68172 | down | LOC644774 | PREDICTED: Homo sapiens similar |
| | | | to Phosphoglycerate kinase 1 |
| | | | (LOC644774), mRNA. |
| 2.675767 | down | ZIC2 | Homo sapiens Zic family member 2 |
| | | | (odd-paired homolog, Drosophila) |
| | | | (ZIC2), mRNA. |
| 2.6746 | down | NPIP | Homo sapiens nuclear pore complex |
| | | | interacting protein (NPIP), mRNA. |
| 2.671463 | down | SSR2 | Homo sapiens signal sequence |
| | | | receptor, beta (translocon-associated |
| | | | protein beta) (SSR2), mRNA. |
| 2.663006 | down | LGALS3BP | Homo sapiens lectin, galactoside- |
| | | | binding, soluble, 3 binding protein |
| | | | (LGALS3BP), mRNA. |
| 2.642011 | down | TSPO | Homo sapiens translocator protein |
| | | | (18 kDa) (TSPO), transcript variant |
| | | | PBR-S, mRNA. |
| 2.637904 | down | LOC3 87867 | PREDICTED: Homo sapiens similar |
| | | | to 40S ribosomal protein SA (p40) |
| | | | (34/67 kDa laminin receptor) (Colon |
| | | | carcinoma laminin-binding protein) |
| | | | (NEM/1CHD4) (Multidrug resistance- |
| | | | associated protein MGr1-Ag) |
| | | | (LOC387867), mRNA. |
| 2.63311 | down | NDUFA4L2 | Homo sapiens NADH dehydrogenase |
| | | | (ubiquinone) 1 alpha subcomplex, 4- |
| | | | like 2 (NDUFA4L2), mRNA. |
| 2.631414 | down | GREM1 | Homo sapiens gremlin 1, cysteine |
| | | | knot superfamily, homolog |
| | | | (Xenopus laevis) (GREM1), mRNA. |
| 2.62583 | down | LOC728732 | PREDICTED: Homo sapiens |
| | | | misc_RNA (LOC728732), miscRNA. |
| 2.623359 | down | SPAG9 | Homo sapiens sperm associated |
| | | | antigen 9 (SPAG9), mRNA. |
| 2.606464 | down | TH | Homo sapiens tyrosine hydroxylase |
| | | | (TH), transcript variant 2, mRNA. |
| 2.604067 | down | MPST | Homo sapiens mercaptopyruvate |
| | | | sulfurtransferase (MPST), nuclear |
| | | | gene encoding mitochondrial protein, |
| | | | transcript variant 2, mRNA. |
| 2.579552 | down | NPDC1 | Homo sapiens neural proliferation, |
| | | | differentiation and control, 1 |
| | | | (NPDC1), mRNA. |
| 2.572909 | down | ACP1 | Homo sapiens acid phosphatase 1, |
| | | | soluble (ACP1), transcript variant 4, |
| | | | mRNA. |
| 2.565894 | down | ATP2C1 | Homo sapiens ATPase, Ca++ |
| | | | transporting, type 2C, member 1 |
| | | | (ATP2C1), transcript variant 4, |
| | | | mRNA. |
| 2.562114 | down | CASC3 | Homo sapiens cancer susceptibility |
| | | | candidate 3 (CASC3), mRNA. |
| 2.555915 | down | LOC441506 | PREDICTED: Homo sapiens |
| | | | misc_RNA (LOC441506), miscRNA. |
| 2.553831 | down | LOC646531 | PREDICTED: Homo sapiens similar |
| | | | to nuclease sensitive element binding |
| | | | protein 1 (LOC646531), mRNA. |
| 2.541955 | down | PQBP1 | Homo sapiens polyglutamine binding |
| | | | protein 1 (PQBP1), transcript variant |
| | | | 3, mRNA. |
| 2.536753 | down | LOC100008589 | Homo sapiens 28S ribosomal RNA |
| | | | (LOC100008589), non-coding RNA. |
| 2.533471 | down | LOC100128771 | PREDICTED: Homo sapiens |
| | | | misc_RNA (LOC100128771), |
| | | | miscRNA. |
| 2.524265 | down | B3GNT6 | Homo sapiens UDP-GlcNAc:betaGal |
| | | | beta-1,3-N- |
| | | | acetylglucosaminyltransferase 6 |
| | | | (B3GNT6), mRNA. |
| 2.515392 | down | RNF5P1 | PREDICTED: Homo sapiens ring |
| | | | finger protein 5 pseudogene 1 |
| | | | (RNF5P1), misc RNA. |
| 2.512245 | down | LOC153561 | PREDICTED: Homo sapiens |
| | | | hypothetical protein LOC153561 |
| | | | (LOC153561), mRNA. |
| 2.511474 | down | NUMA1 | Homo sapiens nuclear mitotic |
| | | | apparatus protein 1 (NUMA1), |
| | | | mRNA. |
| 2.506295 | down | NXPH1 | Homo sapiens neurexophilin 1 |
| | | | (NXPH1), mRNA. |
| 2.499662 | down | RELN | Homo sapiens reelin (RELN), |
| | | | transcript variant 1, mRNA. |
| 2.495256 | down | SNORA67 | Homo sapiens small nucleolar RNA, |
| | | | H/ACA box 67 (SNORA67), small |
| | | | nucleolar RNA. |
| 2.49162 | down | TTC8 | Homo sapiens tetratricopeptide repeat |
| | | | domain 8 (TTC8), transcript variant 1, |
| | | | mRNA. |
| 2.489073 | down | NFKBIA | Homo sapiens nuclear factor of kappa |
| | | | light polypeptide gene enhancer in B- |
| | | | cells inhibitor, alpha (NFKBIA), |
| | | | mRNA. |
| 2.478279 | down | SPTBN1 | Homo sapiens spectrin, beta, non- |
| | | | erythrocytic 1 (SPTBN1), transcript |
| | | | variant 1, mRNA. |
| 2.476282 | down | | Homo sapiens cDNA FLJ45619 fis, |
| | | | clone BRTHA3027318 |
| 2.466702 | down | LOC100132394 | PREDICTED: Homo sapiens |
| | | | hypothetical protein LOC100132394 |
| | | | (LOC100132394), mRNA. |
| 2.465765 | down | GAB2 | Homo sapiens GRB2-associated |
| | | | binding protein 2 (GAB2), transcript |
| | | | variant 1, mRNA. |
| 2.463514 | down | LOC652900 | PREDICTED: Homo sapiens similar |
| | | | to SEZ6L2 protein (LOC652900), |
| | | | mRNA. |
| 2.459292 | down | GLCCI1 | Homo sapiens glucocorticoid induced |
| | | | transcript 1 (GLCCI1), mRNA. |
| 2.455777 | down | CKAP5 | Homo sapiens cytoskeleton associated |
| | | | protein 5 (CKAP5), transcript variant |
| | | | 1, mRNA. |
| 2.450269 | down | LOC388707 | PREDICTED: Homo sapiens |
| | | | misc_RNA (LOC3 88707), miscRNA. |
| 2.446437 | down | SNRPN | Homo sapiens small nuclear |
| | | | ribonucleoprotein polypeptide N |
| | | | (SNRPN), transcript variant 3, mRNA. |
| 2.446257 | down | SMA5 | Homo sapiens SMA5 (SMA5), |
| | | | mRNA. |
| 2.439987 | down | CNBP | Homo sapiens CCHC-type zinc finger, |
| | | | nucleic acid binding protein (CNBP), |
| | | | mRNA. |
| 2.437716 | down | MYT1L | Homo sapiens myelin transcription |
| | | | factor 1-like (MYT1L), mRNA. |
| 2.435687 | down | LOC100128266 | PREDICTED: Homo sapiens |
| | | | misc_RNA (LOC100128266), |
| | | | miscRNA. |
| 2.434372 | down | CD276 | Homo sapiens CD276 molecule |
| | | | (CD276), transcript variant 1, mRNA. |
| | | | XM_945872 XM_945874 |
| 2.429116 | down | PHB2 | Homo sapiens prohibitin 2 (PHB2), |
| | | | transcript variant 2, mRNA. |
| 2.421669 | down | HDGF2 | Homo sapiens hepatoma-derived |
| | | | growth factor-related protein 2 |
| | | | (HDGF2), transcript variant 1, mRNA. |
| 2.410991 | down | FLJ22184 | Homo sapiens hypothetical protein |
| | | | FLJ22184 (FLJ22184), mRNA. |
| 2.407718 | down | SCARB1 | Homo sapiens scavenger receptor |
| | | | class B, member 1 (SCARB1), |
| | | | mRNA. |
| 2.405512 | down | RBMX | Homo sapiens RNA binding motif |
| | | | protein, X-linked (RBMX), mRNA. |
| 2.404951 | down | MBTPS1 | Homo sapiens membrane-bound |
| | | | transcription factor peptidase, site 1 |
| | | | (MBTPS1), mRNA. |
| 2.395497 | down | TMOD1 | Homo sapiens tropomodulin 1 |
| | | | (TMOD1), mRNA. |
| 2.394954 | down | LOC441013 | PREDICTED: Homo sapiens |
| | | | misc_RNA (LOC441013), miscRNA. |
| 2.394569 | down | LOC643531 | PREDICTED: Homo sapiens |
| | | | misc_RNA (LOC643531), miscRNA. |
| 2.385242 | down | MIR1978 | Homo sapiens microRNA 1978 |
| | | | (MIR1978), microRNA. |
| 2.384901 | down | ATN1 | Homo sapiens atrophin 1 (ATN1), |
| | | | transcript variant 1, mRNA. |
| 2.383114 | down | FBLN1 | Homo sapiens fibulin 1 (FBLN1), |
| | | | transcript variant A, mRNA. |
| 2.373436 | down | GUSBL1 | Homo sapiens glucuronidase, beta-like |
| | | | 1 (GUSBL1), non-coding RNA. |
| 2.36863 | down | BIN1 | Homo sapiens bridging integrator 1 |
| | | | (BIN1), transcript variant 1, mRNA. |
| 2.366588 | down | CAMKV | Homo sapiens CaM kinase-like |
| | | | vesicle-associated (CAMKV), mRNA. |
| 2.366183 | down | LOC728658 | PREDICTED: Homo sapiens similar |
| | | | to 23 kD highly basic protein, |
| | | | transcript variant 1 (LOC728658), |
| | | | mRNA. |
| 2.363276 | down | LOC440349 | PREDICTED: Homo sapiens similar |
| | | | to nuclear pore complex interacting |
| | | | protein, transcript variant 1 |
| | | | (LOC440349), mRNA. |
| 2.360442 | down | HDAC9 | Homo sapiens histone deacetylase 9 |
| | | | (HDAC9), transcript variant 3, |
| | | | mRNA. |
| 2.356692 | down | SMA4 | Homo sapiens SMA4 (SMA4), |
| | | | mRNA. |
| 2.346119 | down | UNC5A | Homo sapiens unc-5 homolog A |
| | | | (C. elegans) (UNC5A), mRNA. |
| 2.345477 | down | LOC390354 | PREDICTED: Homo sapiens similar |
| | | | to ribosomal protein L18a; 60S |
| | | | ribosomal protein L18a, transcript |
| | | | variant 36 (LOC390354), misc RNA. |
| 2.336991 | down | UNG | Homo sapiens uracil-DNA |
| | | | glycosylase (UNG), nuclear gene |
| | | | encoding mitochondrial protein, |
| | | | transcript variant 1, mRNA. |
| 2.332026 | down | PRMT1 | Homo sapiens protein arginine |
| | | | methyltransferase 1 (PRMT1), |
| | | | transcript variant 3, mRNA. |
| 2.330827 | down | FTL | Homo sapiens ferritin, light |
| | | | polypeptide (FTL), mRNA. |
| 2.329446 | down | 3-Sep | Homo sapiens septin 3 (SEPT3), |
| | | | transcript variant B, mRNA. |
| 2.325651 | down | ATCAY | Homo sapiens ataxia, cerebellar, |
| | | | Cayman type (caytaxin) (ATCAY), |
| | | | mRNA. |
| 2.322724 | down | PYCR1 | Homo sapiens pyrroline-5-carboxylate |
| | | | reductase 1 (PYCR1), transcript |
| | | | variant 2, mRNA. |
| 2.322228 | down | RANBP1 | Homo sapiens RAN binding protein 1 |
| | | | (RANBP1), mRNA. |
| 2.320466 | down | GNG4 | Homo sapiens guanine nucleotide |
| | | | binding protein (G protein), gamma 4 |
| | | | (GNG4), transcript variant 2, mRNA. |
| 2.317229 | down | TAGLN2 | Homo sapiens transgelin 2 |
| | | | (TAGLN2), mRNA. |
| 2.313841 | down | LOC440157 | Homo sapiens hypothetical gene |
| | | | supported by AK096951; BC066547 |
| | | | (LOC440157), mRNA. |
| 2.30987 | down | CUEDC2 | Homo sapiens CUE domain |
| | | | containing 2 (CUEDC2), mRNA. |
| 2.307863 | down | NFIX | Homo sapiens nuclear factor I/X |
| | | | (CCAAT-binding transcription factor) |
| | | | (NFIX), mRNA. |
| 2.307834 | down | TH1L | Homo sapiens TH1-like (Drosophila) |
| | | | (TH1L), transcript variant 2, mRNA. |
| 2.306791 | down | | AGENCOURT_14354957 |
| | | | NIH_MGC_191 Homo sapiens cDNA |
| | | | clone IMAGE: 30413554 5, mRNA |
| | | | sequence |
| 2.305834 | down | SUMO2 | Homo sapiens SMT3 suppressor of |
| | | | mif two 3 homolog 2 (S. cerevisiae) |
| | | | (SUMO2), transcript variant 2, |
| | | | mRNA. |
| 2.305034 | down | SORL1 | Homo sapiens sortilin-related |
| | | | receptor, L(DLR class) A repeats- |
| | | | containing (SORL1), mRNA. |
| 2.304166 | down | DEAF1 | PREDICTED: Homo sapiens |
| | | | deformed epidermal autoregulatory |
| | | | factor 1 (Drosophila) (DEAF1), |
| | | | mRNA. |
| 2.298846 | down | LOC92755 | PREDICTED: Homo sapiens |
| | | | misc_RNA (LOC92755), miscRNA. |
| 2.294239 | down | CKAP4 | Homo sapiens cytoskeleton-associated |
| | | | protein 4 (CKAP4), mRNA. |
| 2.292942 | down | C12orf24 | Homo sapiens chromosome 12 open |
| | | | reading frame 24 (C12orf24), mRNA. |
| 2.28912 | down | TUBB4Q | Homo sapiens tubulin, beta |
| | | | polypeptide 4, member Q (TUBB4Q), |
| | | | mRNA. |
| 2.286832 | down | LOC728139 | PREDICTED: Homo sapiens |
| | | | misc_RNA (LOC728139), miscRNA. |
| 2.286763 | down | PRRT2 | Homo sapiens pro line-rich |
| | | | transmembrane protein 2 (PRRT2), |
| | | | mRNA. |
| 2.28539 | down | LOC100130561 | PREDICTED: Homo sapiens similar |
| | | | to high-mobility group (nonhistone |
| | | | chromosomal) protein 1-like 10, |
| | | | transcript variant 2 (LOC100130561), |
| | | | mRNA. |
| 2.284874 | down | | Homo sapiens mRNA; cDNA |
| | | | DKFZp686J0156 (from clone |
| | | | DKFZp686J0156) |
| 2.281905 | down | TACC2 | Homo sapiens transforming, acidic |
| | | | coiled-coil containing protein 2 |
| | | | (TACC2), transcript variant 2, mRNA. |
| 2.27488 | down | MAP1B | Homo sapiens microtubule-associated |
| | | | protein 1B (MAP1B), mRNA. |
| 2.273095 | down | PKMYT1 | Homo sapiens protein kinase, |
| | | | membrane associated |
| | | | tyrosine/threonine 1 (PKMYT1), |
| | | | transcript variant 2, mRNA. |
| 2.272674 | down | UCK2 | Homo sapiens uridine-cytidine kinase |
| | | | 2 (UCK2), mRNA. |
| 2.272521 | down | LOC652489 | PREDICTED: Homo sapiens similar |
| | | | to SMT3 suppressor of mif two 3 |
| | | | homolog 2 (LOC652489), mRNA. |
| 2.271163 | down | IRF2BP2 | Homo sapiens interferon regulatory |
| | | | factor 2 binding protein 2 (IRF2BP2), |
| | | | transcript variant 1, mRNA. |
| 2.270466 | down | EEF1D | Homo sapiens eukaryotic translation |
| | | | elongation factor 1 delta (guanine |
| | | | nucleotide exchange protein) |
| | | | (EEF1D), transcript variant 1, mRNA. |
| 2.269964 | down | RALY | Homo sapiens RNA binding protein, |
| | | | autoantigenic (hnRNP-associated with |
| | | | lethal yellow homolog (mouse)) |
| | | | (RALY), transcript variant 2, mRNA. |
| 2.269945 | down | PFKP | Homo sapiens phosphofructokinase, |
| | | | platelet (PFKP), mRNA. |
| 2.265796 | down | CCDC136 | Homo sapiens coiled-coil domain |
| | | | containing 136 (CCDC136), mRNA. |
| 2.262251 | down | RNF165 | Homo sapiens ring finger protein 165 |
| | | | (RNF165), mRNA. |
| 2.259489 | down | NOMO1 | Homo sapiens NODAL modulator 1 |
| | | | (NOMO1), mRNA. |
| 2.259268 | down | TCF3 | Homo sapiens transcription factor 3 |
| | | | (E2A immunoglobulin enhancer |
| | | | binding factors E12/E47) (TCF3), |
| | | | mRNA. |
| 2.257809 | down | LOC401537 | PREDICTED: Homo sapiens |
| | | | misc_RNA (LOC401537), miscRNA. |
| 2.257748 | down | TNPO1 | Homo sapiens transportin 1 (TNPO1), |
| | | | transcript variant 2, mRNA. |
| 2.254448 | down | ST8SIA2 | Homo sapiens ST8 alpha-N-acetyl- |
| | | | neuraminide alpha-2,8- |
| | | | sialyltransferase 2 (ST8SIA2), mRNA. |
| 2.254105 | down | STMN2 | Homo sapiens stathmin-like 2 |
| | | | (STMN2), mRNA. |
| 2.250269 | down | APIP | Homo sapiens APAF1 interacting |
| | | | protein (APIP), mRNA. |
| 2.243515 | down | ATP1A1 | Homo sapiens ATPase, Na+/K+ |
| | | | transporting, alpha 1 polypeptide |
| | | | (ATP1A1), transcript variant 2, |
| | | | mRNA. |
| 2.241412 | down | LOC649150 | PREDICTED: Homo sapiens similar |
| | | | to eukaryotic translation elongation |
| | | | factor 1 alpha 2 (LOC649150), |
| | | | mRNA. |
| 2.240263 | down | PKD1 | Homo sapiens polycystic kidney |
| | | | disease 1 (autosomal dominant) |
| | | | (PKD1), transcript variant 1, mRNA. |
| 2.236215 | down | LOC643300 | PREDICTED: Homo sapiens similar |
| | | | to 60 kDa heat shock protein, |
| | | | mitochondrial precursor (Hsp60) (60 |
| | | | kDa chaperonin) (CPN60) (Heat |
| | | | shock protein 60) (HSP-60) |
| | | | (Mitochondrial matrix protein P1) |
| | | | (P60 lymphocyte protein) (HuCHA60) |
| | | | (LOC643300), mRNA. |
| 2.234636 | down | PLOD3 | Homo sapiens procollagen-lysine, 2- |
| | | | oxoglutarate 5-dioxygenase 3 |
| | | | (PLOD3), mRNA. |
| 2.233062 | down | SDHA | Homo sapiens succinate |
| | | | dehydrogenase complex, subunit A, |
| | | | flavoprotein (Fp) (SDHA), nuclear |
| | | | gene encoding mitochondrial protein, |
| | | | mRNA. |
| 2.230375 | down | GPX7 | Homo sapiens glutathione peroxidase |
| | | | 7 (GPX7), mRNA. |
| 2.226073 | down | THOC4 | PREDICTED: Homo sapiens THO |
| | | | complex 4 (THOC4), mRNA. |
| 2.224401 | down | PRRX2 | Homo sapiens paired related |
| | | | homeobox 2 (PRRX2), mRNA. |
| 2.224325 | down | SGPP2 | PREDICTED: Homo sapiens |
| | | | sphingosine-1-phosphate phosphotase |
| | | | 2 (SGPP2), mRNA. |
| 2.223073 | down | APEX1 | Homo sapiens APEX nuclease |
| | | | (multifunctional DNA repair enzyme) |
| | | | 1 (APEX1), transcript variant 1, |
| | | | mRNA. |
| 2.222308 | down | PHF2 | Homo sapiens PHD finger protein 2 |
| | | | (PHF2), mRNA. |
| 2.221914 | down | CABC1 | Homo sapiens chaperone, ABC1 |
| | | | activity of bc1 complex homolog |
| | | | (S. pombe) (CABC1), nuclear gene |
| | | | encoding mitochondrial protein, |
| | | | mRNA. |
| 2.220262 | down | LOC100134241 | PREDICTED: Homo sapiens |
| | | | hypothetical protein LOC100134241 |
| | | | (LOC100134241), mRNA. |
| 2.218515 | down | LOC732007 | PREDICTED: Homo sapiens similar |
| | | | to Phosphoglycerate mutase 1 |
| | | | (Phosphoglycerate mutase isozyme B) |
| | | | (PGAM-B) (BPG-dependent PGAM |
| | | | 1) (LOC732007), mRNA. |
| 2.211864 | down | CCT6A | Homo sapiens chaperonin containing |
| | | | TCP1, subunit 6A (zeta 1) (CCT6A), |
| | | | transcript variant 1, mRNA. |
| 2.208987 | down | FTL | Homo sapiens ferritin, light |
| | | | polypeptide (FTL), mRNA. |
| 2.208881 | down | THOC3 | Homo sapiens THO complex 3 |
| | | | (THOC3), mRNA. |
| 2.207744 | down | PRR7 | Homo sapiens proline rich 7 (synaptic) |
| | | | (PRR7), mRNA. |
| 2.207275 | down | MCM2 | Homo sapiens minichromosome |
| | | | maintenance complex component 2 |
| | | | (MCM2), mRNA. |
| 2.203152 | down | C9orf86 | Homo sapiens chromosome 9 open |
| | | | reading frame 86 (C9orf86), mRNA. |
| 2.201281 | down | CSNK1E | Homo sapiens casein kinase 1, epsilon |
| | | | (CSNK1E), transcript variant 1, |
| | | | mRNA. |
| 2.199816 | down | MGAT3 | Homo sapiens mannosyl (beta-1,4-)- |
| | | | glycoprotein beta-1,4-N- |
| | | | acetylglucosaminyltransferase |
| | | | (MGAT3), transcript variant 1, |
| | | | mRNA. |
| 2.19858 | down | FEZ1 | Homo sapiens fasciculation and |
| | | | elongation protein zeta 1 (zygin I) |
| | | | (FEZ1), transcript variant 2, mRNA. |
| 2.198326 | down | PODXL2 | Homo sapiens podocalyxin-like 2 |
| | | | (PODXL2), mRNA. |
| 2.194836 | down | ENO2 | Homo sapiens enolase 2 (gamma, |
| | | | neuronal) (ENO2), mRNA. |
| 2.193517 | down | LMO3 | Homo sapiens LIM domain only 3 |
| | | | (rhombotin-like 2) (LMO3), transcript |
| | | | variant 1, mRNA. |
| 2.191548 | down | WDR5 | Homo sapiens WD repeat domain 5 |
| | | | (WDR5), transcript variant 1, mRNA. |
| 2.190587 | down | LOC399804 | PREDICTED: Homo sapiens |
| | | | misc_RNA (LOC399804), miscRNA. |
| 2.190034 | down | PKM2 | Homo sapiens pyruvate kinase, muscle |
| | | | (PKM2), transcript variant 2, mRNA. |
| 2.18845 | down | PLEKHG3 | Homo sapiens pleckstrin homology |
| | | | domain containing, family G (with |
| | | | RhoGef domain) member 3 |
| | | | (PLEKHG3), mRNA. |
| 2.187572 | down | PLD6 | Homo sapiens phospholipase D |
| | | | family, member 6 (PLD6), mRNA. |
| 2.187389 | down | B4GALNT4 | Homo sapiens beta-1,4-N-acetyl- |
| | | | galactosaminyl transferase 4 |
| | | | (B4GALNT4), mRNA. |
| 2.185219 | down | GUSBL1 | Homo sapiens glucuronidase, beta-like |
| | | | 1 (GUSBL1), mRNA. |
| 2.18114 | down | PCBP4 | Homo sapiens poly(rC) binding |
| | | | protein 4 (PCBP4), transcript variant |
| | | | 1, mRNA. |
| 2.177992 | down | C12orf57 | Homo sapiens chromosome 12 open |
| | | | reading frame 57 (C12orf57), mRNA. |
| 2.17742 | down | LOC651198 | PREDICTED: Homo sapiens similar |
| | | | to hCG2036706 (LOC651198), |
| | | | mRNA. |
| 2.173998 | down | GAPDH | Homo sapiens glyceraldehyde-3- |
| | | | phosphate dehydrogenase (GAPDH), |
| | | | mRNA. |
| 2.173798 | down | LOC402251 | PREDICTED: Homo sapiens similar |
| | | | to eukaryotic translation elongation |
| | | | factor 1 alpha 2 (LOC402251), |
| | | | mRNA. |
| 2.169406 | down | PALM | Homo sapiens paralemmin (PALM), |
| | | | transcript variant 1, mRNA. |
| 2.168301 | down | PCK2 | Homo sapiens phosphoenolpyruvate |
| | | | carboxykinase 2 (mitochondrial) |
| | | | (PCK2), nuclear gene encoding |
| | | | mitochondrial protein, transcript |
| | | | variant 1, mRNA. |
| 2.167464 | down | ACO2 | Homo sapiens aconitase 2, |
| | | | mitochondrial (ACO2), nuclear gene |
| | | | encoding mitochondrial protein, |
| | | | mRNA. |
| 2.166496 | down | TIAL1 | Homo sapiens TIA1 cytotoxic |
| | | | granule-associated RNA binding |
| | | | protein-like 1 (TIAL1), transcript |
| | | | variant 2, mRNA. |
| 2.165684 | down | PTPRD | Homo sapiens protein tyrosine |
| | | | phosphatase, receptor type, D |
| | | | (PTPRD), transcript variant 2, mRNA. |
| 2.165212 | down | MARCKSL1 | Homo sapiens MARCKS-like 1 |
| | | | (MARCKSL1), mRNA. |
| 2.163887 | down | 3-Sep | Homo sapiens septin 3 (SEPT3), |
| | | | transcript variant B, mRNA. |
| 2.161171 | down | PISD | Homo sapiens phosphatidylserine |
| | | | decarboxylase (PISD), mRNA. |
| 2.160368 | down | PTK7 | Homo sapiens PTK7 protein tyrosine |
| | | | kinase 7 (PTK7), transcript variant |
| | | | PTK7-4, mRNA. |
| 2.157614 | down | FAF1 | Homo sapiens Fas (TNFRSF6) |
| | | | associated factor 1 (FAF1), mRNA. |
| 2.15675 | down | SLC35F3 | PREDICTED: Homo sapiens solute |
| | | | carrier family 35, member F3 |
| | | | (SLC35F3), mRNA. |
| 2.156618 | down | H2AFX | Homo sapiens H2A histone family, |
| | | | member X (H2AFX), mRNA. |
| 2.156176 | down | GNL3 | Homo sapiens guanine nucleotide |
| | | | binding protein-like 3 (nucleolar) |
| | | | (GNL3), transcript variant 3, mRNA. |
| 2.153096 | down | FAM57B | Homo sapiens family with sequence |
| | | | similarity 57, member B (FAM57B), |
| | | | mRNA. |
| 2.151984 | down | CDK5R1 | Homo sapiens cyclin-dependent |
| | | | kinase 5, regulatory subunit 1 (p35) |
| | | | (CDK5R1), mRNA. |
| 2.150896 | down | TNIP1 | Homo sapiens TNFAIP3 interacting |
| | | | protein 1 (TNIP1), mRNA. |
| 2.150337 | down | EEF1D | Homo sapiens eukaryotic translation |
| | | | elongation factor 1 delta (guanine |
| | | | nucleotide exchange protein) |
| | | | (EEF1D), transcript variant 1, mRNA. |
| 2.149941 | down | TRPC4AP | Homo sapiens transient receptor |
| | | | potential cation channel, subfamily C, |
| | | | member 4 associated protein |
| | | | (TRPC4AP), transcript variant 1, |
| | | | mRNA. |
| 2.147648 | down | RAD51AP1 | Homo sapiens RAD51 associated |
| | | | protein 1 (RAD51AP1), mRNA. |
| 2.144922 | down | PSCD1 | Homo sapiens pleckstrin homology, |
| | | | Sec7 and coiled-coil domains |
| | | | 1(cytohesin 1) (PSCD1), transcript |
| | | | variant 2, mRNA. |
| 2.141083 | down | RELN | Homo sapiens reelin (RELN), |
| | | | transcript variant 2, mRNA. |
| 2.139602 | down | SIGMAR1 | Homo sapiens sigma non-opioid |
| | | | intracellular receptor 1 (SIGMAR1), |
| | | | transcript variant 1, mRNA. |
| 2.135113 | down | STXBP1 | Homo sapiens syntaxin binding |
| | | | protein 1 (STXBP1), transcript variant |
| | | | 2, mRNA. |
| 2.134394 | down | LOC643873 | PREDICTED: Homo sapiens |
| | | | misc_RNA (LOC643873), miscRNA. |
| 2.134312 | down | SKP2 | Homo sapiens S-phase kinase- |
| | | | associated protein 2 (p45) (SKP2), |
| | | | transcript variant 1, mRNA. |
| 2.131977 | down | HNRPK | Homo sapiens heterogeneous nuclear |
| | | | ribonucleoprotein K (HNRPK), |
| | | | transcript variant 3, mRNA. |
| 2.131822 | down | FEZ1 | Homo sapiens fasciculation and |
| | | | elongation protein zeta 1 (zygin I) |
| | | | (FEZ1), transcript variant 1, mRNA. |
| 2.129834 | down | HNRNPL | Homo sapiens heterogeneous nuclear |
| | | | ribonucleoprotein L (HNRNPL), |
| | | | transcript variant 2, mRNA. |
| 2.129226 | down | ADM | Homo sapiens adrenomedullin |
| | | | (ADM), mRNA. |
| 2.126514 | down | DBNDD2 | Homo sapiens dysbindin (dystrobrevin |
| | | | binding protein 1) domain containing |
| | | | 2 (DBNDD2), transcript variant 3, |
| | | | mRNA. |
| 2.125708 | down | LOC643668 | PREDICTED: Homo sapiens similar |
| | | | to peptidase (prosome, macropain) |
| | | | 26S subunit, ATPase 1 (LOC643668), |
| | | | mRNA. |
| 2.125221 | down | NGFRAP1 | Homo sapiens nerve growth factor |
| | | | receptor (TNFRSF16) associated |
| | | | protein 1 (NGFRAP1), transcript |
| | | | variant 3, mRNA. |
| 2.124531 | down | FOXK1 | Homo sapiens forkhead box K1 |
| | | | (FOXK1), mRNA. |
| 2.124004 | down | CENTG3 | Homo sapiens centaurin, gamma 3 |
| | | | (CENTG3), mRNA. |
| 2.123406 | down | NME3 | Homo sapiens non-metastatic cells 3, |
| | | | protein expressed in (NME3), mRNA. |
| 2.12233 | down | EIF4A1 | Homo sapiens eukaryotic translation |
| | | | initiation factor 4A, isoform 1 |
| | | | (EIF4A1), mRNA. |
| 2.121922 | down | LOC100131735 | PREDICTED: Homo sapiens |
| | | | misc_RNA (LOC100131735), |
| | | | miscRNA. |
| 2.120917 | down | SAC3D1 | Homo sapiens SAC3 domain |
| | | | containing 1 (SAC3D1), mRNA. |
| 2.120508 | down | LOC100134364 | PREDICTED: Homo sapiens |
| | | | hypothetical protein LOC100134364 |
| | | | (LOC100134364), mRNA. |
| 2.119961 | down | TMSB10 | Homo sapiens thymosin beta 10 |
| | | | (TMSB10), mRNA. |
| 2.119585 | down | IDH2 | Homo sapiens isocitrate |
| | | | dehydrogenase 2 (NADP+), |
| | | | mitochondrial (IDH2), nuclear gene |
| | | | encoding mitochondrial protein, |
| | | | mRNA. |
| 2.117743 | down | DPM3 | Homo sapiens dolichyl-phosphate |
| | | | mannosyltransferase polypeptide 3 |
| | | | (DPM3), transcript variant 2, mRNA. |
| 2.117534 | down | PRKCZ | Homo sapiens protein kinase C, zeta |
| | | | (PRKCZ), transcript variant 1, mRNA. |
| 2.117004 | down | EIF4H | Homo sapiens eukaryotic translation |
| | | | initiation factor 4H (EIF4H), transcript |
| | | | variant 2, mRNA. |
| 2.115663 | down | GAS6 | Homo sapiens growth arrest-specific 6 |
| | | | (GAS6), mRNA. |
| 2.115582 | down | NHP2 | Homo sapiens NHP2 |
| | | | ribonucleoprotein homolog (yeast) |
| | | | (NHP2), transcript variant 1, mRNA. |
| 2.110555 | down | CNTFR | Homo sapiens ciliary neurotrophic |
| | | | factor receptor (CNTFR), transcript |
| | | | variant 2, mRNA. |
| 2.104631 | down | LOC440927 | PREDICTED: Homo sapiens similar |
| | | | to 60S acidic ribosomal protein P1, |
| | | | transcript variant 4 (LOC440927), |
| | | | mRNA. |
| 2.10323 | down | LOC286444 | PREDICTED: Homo sapiens |
| | | | misc_RNA (LOC286444), miscRNA. |
| 2.103015 | down | LOC100133840 | PREDICTED: Homo sapiens similar |
| | | | to hCG1994151 (LOC100133840), |
| | | | mRNA. |
| 2.102011 | down | TSC22D3 | Homo sapiens TSC22 domain family, |
| | | | member 3 (TSC22D3), transcript |
| | | | variant 1, mRNA. |
| 2.101288 | down | | Homo sapiens mRNA; cDNA |
| | | | DKFZp686E0389 (from clone |
| | | | DKFZp686E0389) |
| 2.096581 | down | KIAA0195 | Homo sapiens KIAA0195 |
| | | | (KIAA0195), mRNA. |
| 2.095432 | down | LOC728873 | PREDICTED: Homo sapiens |
| | | | misc_RNA (LOC728873), miscRNA. |
| 2.094849 | down | BIN1 | Homo sapiens bridging integrator 1 |
| | | | (BIN1), transcript variant 4, mRNA. |
| 2.088547 | down | RSL1D1 | Homo sapiens ribosomal L1 domain |
| | | | containing 1 (RSL1D1), mRNA. |
| 2.08493 | down | N4BP2L1 | Homo sapiens NEDD4 binding |
| | | | protein 2-like 1 (N4BP2L1), transcript |
| | | | variant 2, mRNA. |
| 2.079067 | down | NIPSNAP1 | Homo sapiens nipsnap homolog 1 |
| | | | (C. elegans) (NIPSNAP1), mRNA. |
| 2.078163 | down | GPSM1 | PREDICTED: Homo sapiens G- |
| | | | protein signalling modulator 1 (AGS3- |
| | | | like, C. elegans) (GPSM1), mRNA. |
| 2.077028 | down | COLEC11 | Homo sapiens collectin sub-family |
| | | | member 11 (COLEC11), transcript |
| | | | variant 2, mRNA. |
| 2.074221 | down | TNC | Homo sapiens tenascin C |
| | | | (hexabrachion) (TNC), mRNA. |
| 2.073396 | down | LOC100129585 | PREDICTED: Homo sapiens similar |
| | | | to hCG2011544 (LOC100129585), |
| | | | mRNA. |
| 2.071684 | down | NDUFV1 | Homo sapiens NADH dehydrogenase |
| | | | (ubiquinone) flavoprotein 1, 51 kDa |
| | | | (NDUFV1), mRNA. |
| 2.068496 | down | TPT1 | Homo sapiens tumor protein, |
| | | | translationally-controlled 1 (TPT1), |
| | | | mRNA. |
| 2.067051 | down | ZNF423 | Homo sapiens zinc finger protein 423 |
| | | | (ZNF423), mRNA. |
| 2.066952 | down | UCKL1 | Homo sapiens uridine-cytidine kinase |
| | | | 1-like 1 (UCKL1), mRNA. |
| 2.066395 | down | MDK | Homo sapiens midkine (neurite |
| | | | growth-promoting factor 2) (MDK), |
| | | | transcript variant 1, mRNA. |
| 2.065846 | down | TIGA1 | Homo sapiens TIGA1 (TIGA1), |
| | | | mRNA. |
| 2.064929 | down | LOC727761 | PREDICTED: Homo sapiens similar |
| | | | to Deoxythymidylate kinase |
| | | | (thymidylate kinase), transcript variant |
| | | | 4 (LOC727761), mRNA. |
| 2.064799 | down | FAM125B | Homo sapiens family with sequence |
| | | | similarity 125, member B |
| | | | (FAM125B), transcript variant 1, |
| | | | mRNA. |
| 2.064142 | down | LOC157627 | Homo sapiens hypothetical |
| | | | LOC157627 (LOC157627), non- |
| | | | coding RNA. |
| 2.063894 | down | SDC1 | Homo sapiens syndecan 1 (SDC1), |
| | | | transcript variant 1, mRNA. |
| 2.063298 | down | SLC10A4 | Homo sapiens solute carrier family 10 |
| | | | (sodium/bile acid cotransporter |
| | | | family), member 4 (SLC10A4), |
| | | | mRNA. |
| 2.062089 | down | SCAMP5 | Homo sapiens secretory carrier |
| | | | membrane protein 5 (SCAMP5), |
| | | | mRNA. |
| 2.061974 | down | DAPK1 | Homo sapiens death-associated |
| | | | protein kinase 1 (DAPK1), mRNA. |
| 2.060575 | down | LOC389141 | PREDICTED: Homo sapiens |
| | | | misc_RNA (LOC389141), miscRNA. |
| 2.055651 | down | HRK | Homo sapiens harakiri, BCL2 |
| | | | interacting protein (contains only BH3 |
| | | | domain) (HRK), mRNA. |
| 2.053187 | down | LOC100132060 | PREDICTED: Homo sapiens |
| | | | hypothetical protein LOC100132060 |
| | | | (LOC100132060), mRNA. |
| 2.053184 | down | PNMA3 | Homo sapiens paraneoplastic antigen |
| | | | MA3 (PNMA3), mRNA. |
| 2.051588 | down | DYRK2 | Homo sapiens dual-specificity |
| | | | tyro sine-(Y)-phosphorylation |
| | | | regulated kinase 2 (DYRK2), |
| | | | transcript variant 1, mRNA. |
| 2.051375 | down | MRPS24 | Homo sapiens mitochondrial |
| | | | ribosomal protein S24 (MRPS24), |
| | | | nuclear gene encoding mitochondrial |
| | | | protein, mRNA. |
| 2.051081 | down | LOC648927 | PREDICTED: Homo sapiens |
| | | | misc_RNA (LOC648927), miscRNA. |
| 2.050558 | down | FRZB | Homo sapiens frizzled-related protein |
| | | | (FRZB), mRNA. |
| 2.049056 | down | KLF11 | PREDICTED: Homo sapiens Kruppel- |
| | | | like factor 11 (KLF11), mRNA. |
| 2.048945 | down | LOC644237 | PREDICTED: Homo sapiens |
| | | | misc_RNA (LOC644237), miscRNA. |
| 2.047743 | down | LOC648024 | PREDICTED: Homo sapiens similar |
| | | | to eukaryotic translation initiation |
| | | | factor 4A, isoform 1 (LOC648024), |
| | | | mRNA. |
| 2.04652 | down | TNRC4 | Homo sapiens trinucleotide repeat |
| | | | containing 4 (TNRC4), mRNA. |
| 2.045708 | down | HNRNPK | Homo sapiens heterogeneous nuclear |
| | | | ribonucleoprotein K (HNRNPK), |
| | | | transcript variant 2, mRNA. |
| 2.045596 | down | CALD1 | Homo sapiens caldesmon 1 (CALD1), |
| | | | transcript variant 3, mRNA. |
| 2.041429 | down | PWWP2B | Homo sapiens PWWP domain |
| | | | containing 2B (PWWP2B), transcript |
| | | | variant 1, mRNA. |
| 2.041209 | down | WDR45L | Homo sapiens WDR45-like |
| | | | (WDR45L), mRNA. |
| 2.040227 | down | LOC440595 | PREDICTED: Homo sapiens |
| | | | misc_RNA (LOC440595), miscRNA. |
| 2.039603 | down | HDAC9 | Homo sapiens histone deacetylase 9 |
| | | | (HDAC9), transcript variant 5, |
| | | | mRNA. |
| 2.038931 | down | TRIM28 | Homo sapiens tripartite motif- |
| | | | containing 28 (TRIM28), mRNA. |
| 2.036902 | down | ADAR | Homo sapiens adenosine deaminase, |
| | | | RNA-specific (ADAR), transcript |
| | | | variant 2, mRNA. |
| 2.033741 | down | TMEM101 | Homo sapiens transmembrane protein |
| | | | 101 (TMEM101), mRNA. |
| 2.032995 | down | PEG10 | PREDICTED: Homo sapiens |
| | | | paternally expressed 10 (PEG10), |
| | | | mRNA. |
| 2.032149 | down | HNRNPA3 | Homo sapiens heterogeneous nuclear |
| | | | ribonucleoprotein A3 (HNRNPA3), |
| | | | mRNA. |
| 2.031689 | down | LOC100134648 | PREDICTED: Homo sapiens similar |
| | | | to hCG2024106, transcript variant 2 |
| | | | (LOC100134648), mRNA. |
| 2.029636 | down | LOC728411 | PREDICTED: Homo sapiens similar |
| | | | to Beta-glucuronidase precursor |
| | | | (LOC728411), mRNA. |
| 2.029015 | down | GAPDH | Homo sapiens glyceraldehyde-3- |
| | | | phosphate dehydrogenase (GAPDH), |
| | | | mRNA. |
| 2.028673 | down | GRIA4 | Homo sapiens glutamate receptor, |
| | | | ionotrophic, AMPA 4 (GRIA4), |
| | | | transcript variant 3, mRNA. |
| 2.022971 | down | CACNA1H | Homo sapiens calcium channel, |
| | | | voltage-dependent, T type, alpha 1H |
| | | | subunit (CACNA1H), transcript |
| | | | variant 2, mRNA. |
| 2.020432 | down | SNHG3-RCC1 | Homo sapiens SNHG3-RCC1 |
| | | | readthrough transcript (SNHG3- |
| | | | RCC1), transcript variant 1, mRNA. |
| 2.018636 | down | EEF1A1 | Homo sapiens eukaryotic translation |
| | | | elongation factor 1 alpha 1 (EEF1A1), |
| | | | mRNA. |
| 2.018083 | down | SLC4A2 | Homo sapiens solute carrier family 4, |
| | | | anion exchanger, member 2 |
| | | | (erythrocyte membrane protein band |
| | | | 3-like 1) (SLC4A2), mRNA. |
| 2.015969 | down | TUBB3 | Homo sapiens tubulin, beta 3 |
| | | | (TUBB3), mRNA. |
| 2.01536 | down | PIM1 | Homo sapiens pim-1 oncogene |
| | | | (PIM1), mRNA. |
| 2.014172 | down | ZNRD1 | Homo sapiens zinc ribbon domain |
| | | | containing 1 (ZNRD1), transcript |
| | | | variant a, mRNA. |
| 2.013845 | down | ZNF536 | Homo sapiens zinc finger protein 536 |
| | | | (ZNF536), mRNA. |
| 2.011526 | down | RPL13A | Homo sapiens ribosomal protein L13a |
| | | | (RPL13A), mRNA. |
| 2.010711 | down | DBNDD1 | Homo sapiens dysbindin (dystrobrevin |
| | | | binding protein 1) domain containing |
| | | | 1 (DBNDD1), transcript variant 1, |
| | | | mRNA. |
| 2.009193 | down | TXNDC5 | Homo sapiens thioredoxin domain |
| | | | containing 5 (TXNDC5), transcript |
| | | | variant 2, mRNA. |
| 2.007024 | down | PDZD4 | Homo sapiens PDZ domain containing |
| | | | 4 (PDZD4), mRNA. |
| 2.006589 | down | SLC27A3 | Homo sapiens solute carrier family 27 |
| | | | (fatty acid transporter), member 3 |
| | | | (SLC27A3), mRNA. |
| 2.001083 | down | RPL12 | Homo sapiens ribosomal protein L12 |
| | | | (RPL12), mRNA. |

### Example 15

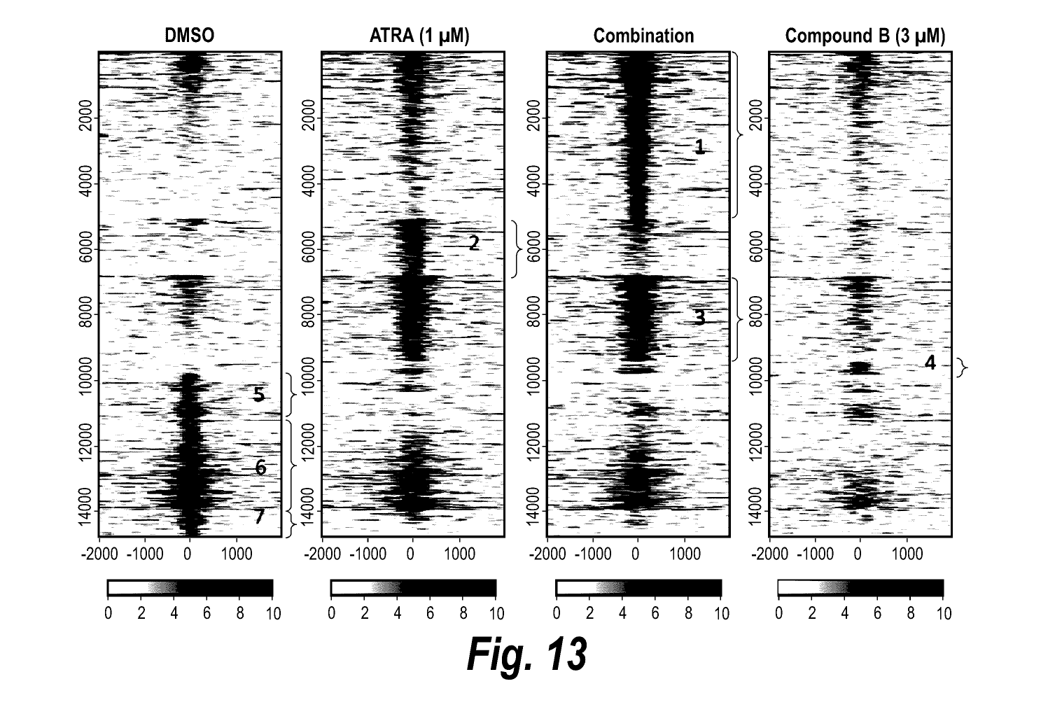

### ATRA Modulates Binding Positions of Retinoic Acid Receptor (RAR) to the Chromatin and is Enhanced by Compound B (FIG. 13)

Retinoic acid receptor active binding sites defined in any individual treatment group by ChIP-seq at 48 hrs after treatment were stacked (y-axis) and aligned to the center of the binding peak (x-axis) (FIG. 13). ATRA treatment caused increased RAR binding (regions 1-4), which was further enhanced by HDAC1/2i across a large proportion of sites (region 1). Treatment also caused RAR binding to decrease (regions 5-7), with potent effects observed in the Compound B single agent group (region 6). Many of the genes found near region 1 binding sites are involved in regulation of retinoid signaling and are transcription factors that drive differentiation (Table 10). Pathway analysis of transcription factors near region 1 retinoic acid receptor binding sites suggest relevant pathways in neuroblastoma differentiation that might be activated (Table 11).

Regions 1-7 of FIG. 13 are defined relative to DMSO: 1, [Combo] Increased; 2, [ATRA] Increased; 3, [ATRA] [Combo] Increased; 4, [Combo] [ATRA] [Compound B] Increased; 5, [Combo] [ATRA] Decreased; 6, [Compound B] Decreased; and 7, [Compound B] [Combo] [ATRA] Decreased.

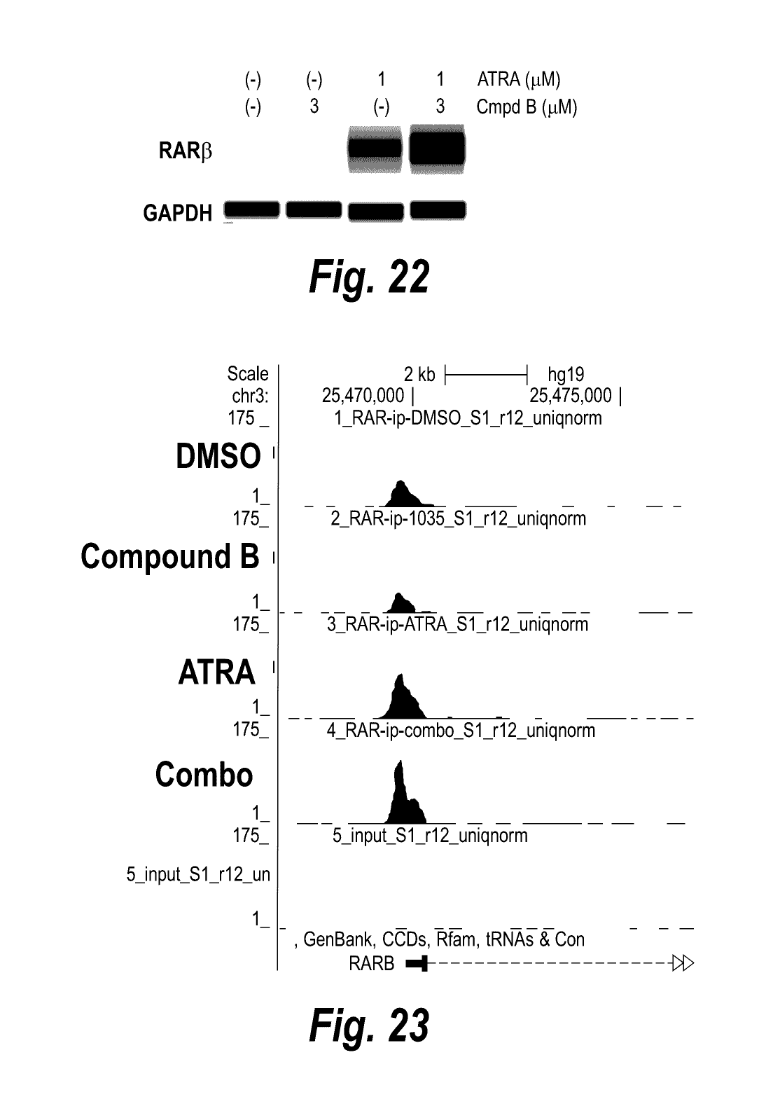

Treatment with HDAC1/2i+RA enhanced RA-induced expression of the RARβ gene (FIG. 21) and increased RARβ protein levels (FIG. 22). Further, RAR binding to the RARβ gene promoter was enhanced in the combination setting relative to either Compound B or RA alone (FIG. 23).

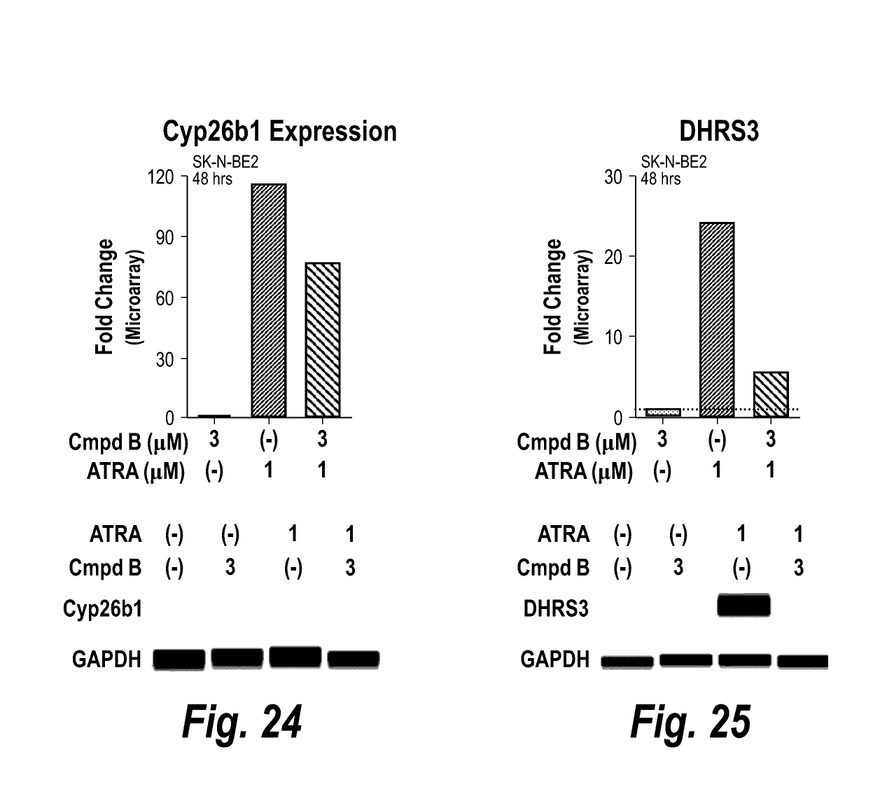

HDAC1/2i inhibits RA-inducible regulators of RAR signaling. Retinoic acid treatment induces a negative feedback loop that regulates RAR signaling. Cyp26b1 and DHRS3, proteins that are induced by RA and negatively regulate RAR signaling, were decreased in the combination setting as measured by gene expression and protein levels (FIG. 24 and FIG. 25).

TABLE 10
Selected Region 1 (FIG. 13) Genes Involved in RA Signaling

| Gene Name | Description |
|---|---|
| HOXA, | A group of related genes that control the body plan of an |
| HOXB, | embryo along the anterior-posterior (head-tail) axis. |
| HOXC, | Coordinated activation is required for differentiation. |
| HOXD | |
| NCOA2, | NCoA is a transcriptional coregulatory protein that contains |
| NCOA3 | several nuclear receptor interacting domains and an intrinsic |
| | histone acetyltransferase activity. |
| | NCOA2 is recruited to DNA promotion sites by ligand- |
| | activated nuclear receptors. NCOA2 in turn acetylates |
| | histones, which makes downstream DNA more accessible |
| | to transcription. |
| RARA, | Retinoic acid receptor isoforms that are activated by |
| RARB | retinoic acid |
| CYP26A1, | cytochrome P450 superfamily of enzymes, which catalyze |
| B1, C1 | many reactions involved in drug metabolism and synthesis |
| | of cholesterol, steroids and other lipids including |
| | retinoids. |
| HDAC3 | Histone deacetylase 3, a component of the Ncor repressive |
| | complex known to interact with RAR, repressing RA |
| | signaling |

TABLE 11
Pathways that Overlap with Region 1 (FIG. 13) Transcription Factors

| Gene Set Name | | # Genes In | | | FDR |
|---|---|---|---|---|---|
| [# Genes (K)] | Description | Overlap (k) | k/K | p-value | q-value |
| HALLMARK_TNFA_ SIGNALING_VIA_NFKB [200] | Genes regulated by NF-kB in response to TNF [GeneID = 7124]. | 13 | 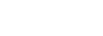 | 1.19 e⁻⁹ | 5.93 e⁻⁸ |
| HALLMARK_G2M_ CHECKPOINT [200] | Genes involved in the G2/M checkpoint, as in progression through the cell division cycle. | 10 | 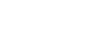 | 1.12 e⁻⁶ | 1.87 e⁻⁵ |
| HALLMARK_TGF_BETA_ SIGNALING [54] | Genes up-regulated in response to TGFB1 [GeneID = 7040]. | 6 | 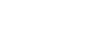 | 1.67 e⁻⁶ | 2.09 e⁻⁵ |
| HALLMARK_HEDGEHOG_ SIGNALING [36] | Genes up-regulated by activation of hedgehog signaling. | 5 | 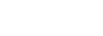 | 4.1 e⁻⁶ | 4.1 e⁻⁵ |
| HALLMARK_P53_PATHWAY [200] | Genes involved in p53 pathways and networks. | 7 | 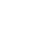 | 4.23 e⁻⁴ | 1.82 e⁻³ |

Additional RAR binding occurs after ATRA is applied. Unexpectedly, combination of ATRA and Compound B increases RAR binding sites relative to ATRA alone. Further, Compound B can reduce RAR binding as a single agent.

### ChIP-Seq Experimental Design

Neuroblastoma cell line SK-N-BE(2) cells were treated with 3 μM Compound B, 1 μM ATRA, or a combination of both at 37° C. over 48 hours and compared to the solvent (DMSO) control. Antibodies to pan-retinoic acid receptor were used to pull down DNA associated with the receptor binding and sequenced. Lists of binding regions were mapped to the chromosome and “associated” with a given gene if the binding region was located within 10,000 bp up- or down-stream of the gene in question. Binding regions were represented as a heatmap where the sites were stacked on the y-axis across treatments, the “0” mark on the x-axis is the center of the binding peak and the intensity of signal indicated by color. The bracketed regions are areas of statistical significance relative to the DMSO control as indicated on the plot.

### Example 16

### Integration of RAR ChIP-Seq and Microarray Data Reveal Potential Drivers of HDACi Enhancement of Retinoid Activity

Table 12 lists the genes returned when the following conditions are met: 1) combo peak height >4 fold relative to the DMSO control group, and 2) combo expression >4 fold relative to the DMSO control group. RAR ChIP-seq and microarray data (48 hr) was queried to identify a list of genes near RAR binding sites that 1) showed enhanced RAR-chromatin interactions and 2) increased gene expression in the combination setting. Functional sorting suggests three key processes are activated: 1) RA metabolism, 2) RA signaling, and 3) kinase signaling.

TABLE 12

| | Published | | | RAR Binding | | | Gene Expression Change | | |
|---|---|---|---|---|---|---|---|---|---|
| | RAR | Known Functions | | (Peak Height) | | | Cmpd. B | ATRA | Combo |
| CYP26A1 | Yes | RA Metabolism | RA | 2.33 | 7.00 | 8.67 | 0.99 | 7.84 | 8.18 |
| CYP26B1 | Yes | RA Metabolism | Processing | 1.50 | 11.50 | 10.00 | 1.87 | 116.67 | 77.45 |
| DHRS3 | Yes | RA Metabolism | | 1.00 | 7.50 | 5.50 | 1.03 | 24.14 | 5.60 |
| CRABP2 | Yes | RA Transport to the Nucleus | RA | 0.69 | 3.15 | 4.23 | 1.18 | 12.15 | 9.49 |
| RARB | Yes | RAR beta Isoform | Signaling | 1.10 | 2.20 | 4.90 | 1.14 | 3.91 | 5.64 |
| PTGER2 | — | RA/ERK1/2 Signaling | | 0.75 | 2.13 | 4.50 | 1.41 | 2.48 | 4.73 |
| ETS1 | Yes | ERK Signaling | Potential | 3.35 | 3.99 | 6.26 | 3.00 | 3.50 | 8.50 |
| IER3 | Yes | ERK Signaling | Non-Genomic | 2.67 | 6.83 | 8.67 | 2.33 | 3.75 | 8.81 |
| RET | Yes | AKT Signaling | RAR | 1.71 | 8.86 | 10.57 | 1.34 | 10.78 | 11.62 |
| NFKBIZ | Yes | AKT/MAPK Regulation | Signaling | 3.33 | 4.00 | 14.67 | 1.95 | 2.85 | 6.33 |
| DUSP6 | Yes | ERK Regulation | | 3.00 | 4.00 | 6.00 | 3.33 | 8.59 | 9.96 |
| CDKN1A | Yes | p21 master cell cycle regulator | Potential | 0.50 | 4.75 | 5.00 | 1.93 | 1.25 | 5.86 |
| PCDH18 | Yes | Cellular Adhesion | Phenotype | 4.00 | 3.50 | 7.00 | 2.53 | 3.98 | 4.37 |
| CTSH | Yes | Lysosomal Function | Drivers | 0.83 | 5.42 | 4.33 | 3.05 | 2.40 | 5.80 |
| ATP7A | Yes | Copper Metal Homeostasis | | 2.20 | 4.60 | 12.40 | 1.98 | 4.30 | 6.80 |
| HSPA5 | — | Protein Synthesis | | 2.00 | 2.00 | 4.86 | 3.78 | 1.48 | 4.55 |
| ACSL3 | Yes | Metabolic Processes | | 2.00 | 1.00 | 9.00 | 2.23 | 1.68 | 4.63 |

### Example 17

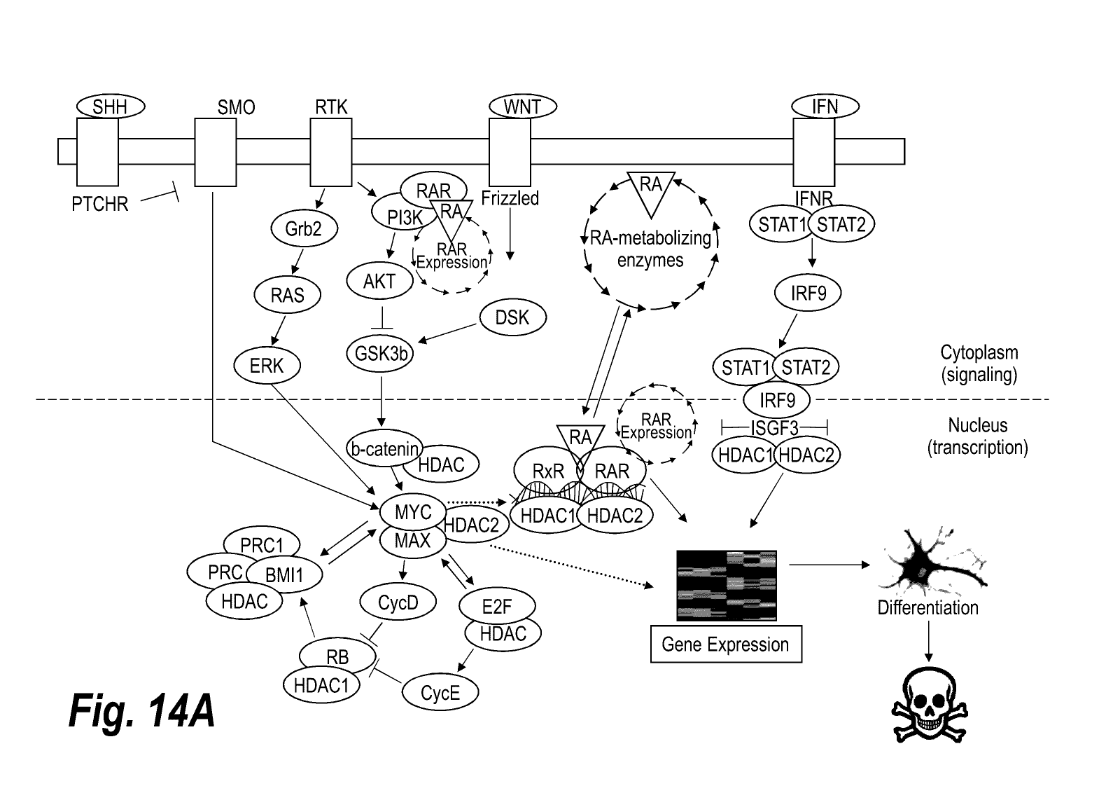

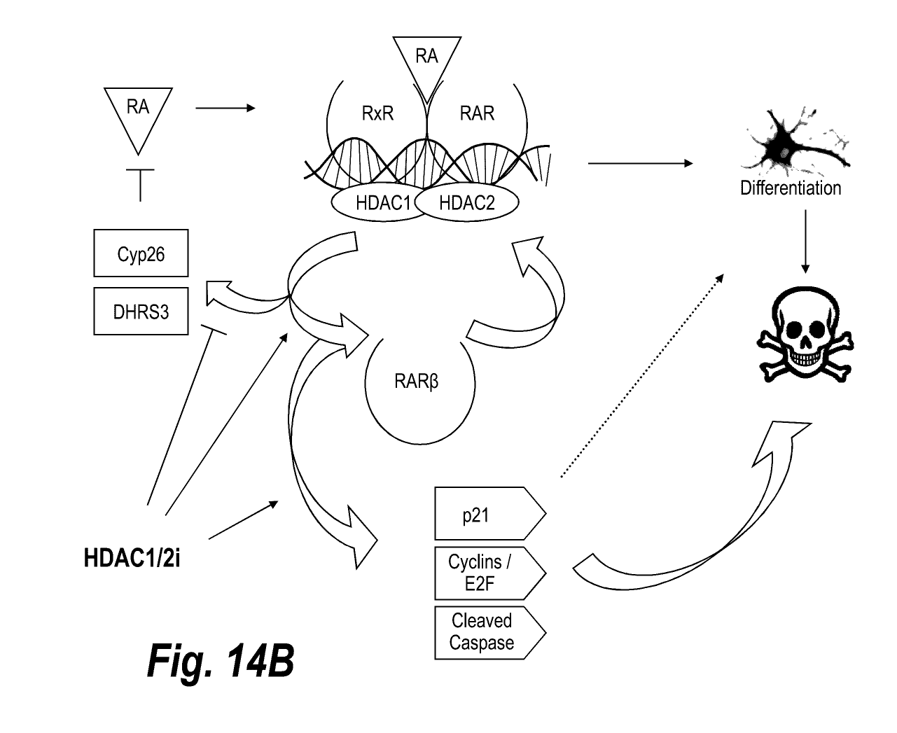

### Model for HDACi Enhancement of RA-Mediated Differentiation (FIG. 14)

A model for HDAC1/2i contribution to retinoid-induced differentiation has emerged from analysis of RAR ChIP-seq and microarray studies (FIG. 14A). The proposed model captures key signaling routes, including the Wnt, RTK and SHH pathways.

Classical metrics of differentiation induced by ATRA are enhanced by HDAC1/2i, which include reduced proliferation, cell cycle effects and dendrite outgrowth. HDAC1/2i has direct anti-tumor effects that are retinoid independent. Gene expression changes consistent with differentiation are induced by HDAC1/2i and are enhanced in combination with retinoic acid. HDAC1/2i modulates RAR interactions with the chromatin near key genes involved in differentiation and cell growth, metabolism and survival.

FIG. 14B represents a proposed model for how HDAC1/2i impacts retinoic acid induced differentiation of neuroblastoma. RA signaling is reinforced through a positive feedback cycle due to an increase in the retinoic acid receptor. Simultaneously, enzymes that limit the intracellular pool of RA are also increased, negatively regulating RA signaling. The homeostatic balance is perturbed by HDAC1/2i+RA combination, resulting in enhanced RAR signaling, decreased levels of enzymes that reduce cellular RA, and slower proliferation by modulating cyclins and inducing p21 while also inducing caspase cleavage. The net result is enhanced differentiation, decreased proliferation, and neuroblastoma cell death

### Example 18

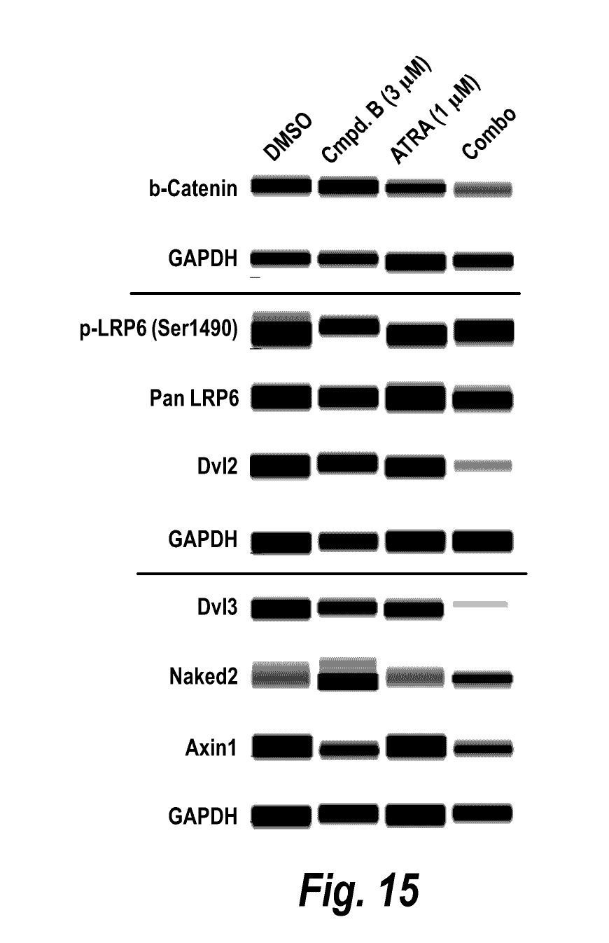

### HDAC1/2 Inhibitors in Combination with ATRA Disrupt the Wnt Signaling Pathway (FIG. 15)

The levels of Wnt-related signaling molecules were assessed following treatment (SK-N-BE2 cells; 3 days in culture) with DMSO (control), Compound B alone, ATRA alone, and Compound B in combination with ATRA. b-catenin, the key signaling molecule in the Wnt pathway, is decreased by the Compound B and ATRA combination. Activated p-LRP is decreased after Compound B single agent treatment. Disheveled (Dvl) 2 & 3 is reduced by Compound B and ATRA combination treatment. Naked2 is increased by Compound B as a single agent and in combination with ATRA. Axin1 is decreased by Compound B as a single agent and in combination with ATRA. Taken together, Compound B in combination with ATRA reduces Wnt signaling.

### Example 19

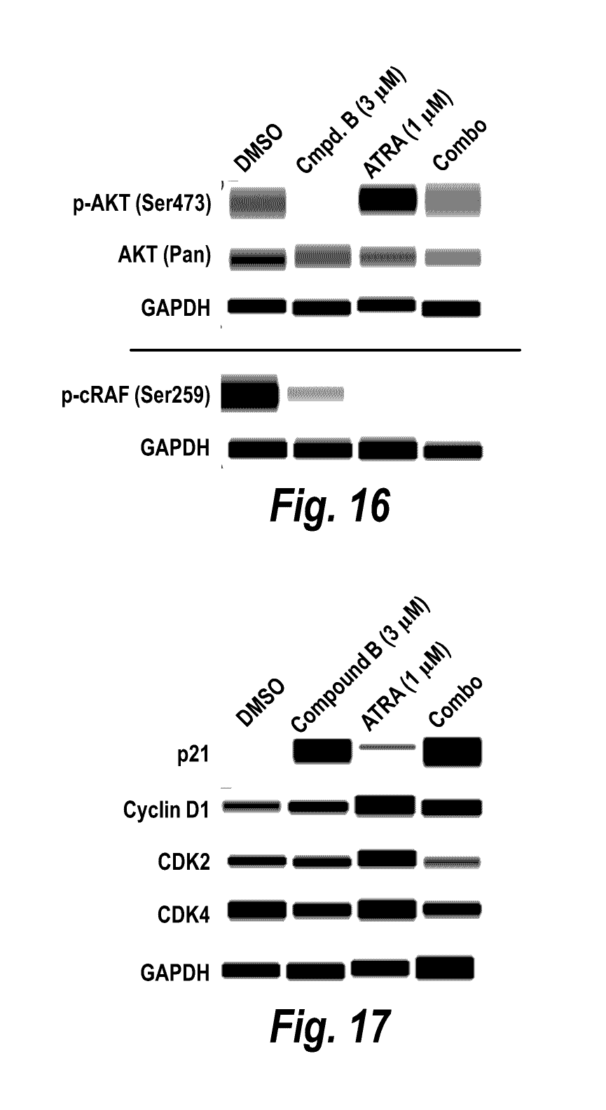

### Retinoic Acid-Activated AKT is Reduced by HDAC1/2i (FIG. 16)

The levels of AKT and c-RAF were assessed following treatment (SK-N-BE2 cells; 3 days in culture) with DMSO (control), Compound B alone, ATRA alone, and Compound B in combination with ATRA. Activated AKT is increased by ATRA and reduced in the combination setting with Compound B. Compound B as a single agent, and in combination with ATRA, decreases cRAF phosphorylation at residue Ser259.

### Example 20

### Modulation of Cell Cycle Progression Through G1 (FIG. 17)

The levels of proteins related to cell-cycle progression were assessed following treatment (SK-N-BE2 cells; 3 days in culture) with DMSO (control), Compound B alone, ATRA alone, and Compound B in combination with ATRA. p21 is increased by ATRA and enhanced by Compound B. Cyclin D1 is decreased in the combination setting relative to ATRA as a single agent. CDK2 and CDK4 are decreased in the combination setting with Compound B and ATRA.

As described herein, the activity of an orally bioavailable HDAC1/2 inhibitor (HDAC1/2i) on neuroblastoma (NB) cell differentiation, proliferation and apoptosis was examined RA combined with HDAC1/2i enhances gene expression patterns associated with differentiation, slows cellular proliferation and more rapidly induces dendrite formation than RA can achieve alone. The mechanisms leading to the differentiated phenotype were examined by microarray and retinoic acid receptor (RAR) ChIP-seq. HDAC1/2i and RA together caused increased localization of the RAR to its own RARα and RARβ promoter regions, and an increase in RAR mRNA and protein relative to the RA treatment condition alone. Additionally, expression of Cyp26a1/b1, enzymes responsible for clearing intercellular RA, were reduced in the combination setting. Gene set enrichment analysis of the microarray data comparing the combination setting against RA as a single agent suggested that the addition of HDAC1/2i was enhancing apoptotic pathways and decreasing E2F driven cell cycle signaling.

In further experiments, enhanced apoptosis was confirmed in the combination setting by measuring caspase 3 and PARP cleavage, which is consistent with reduced proliferation, increased sub-G1 cell frequency in cell cycle assays and ablation of emergent RA-resistant NB colonies. Further, the E2F-activators, CDK4 and CDK6, were reduced at the protein level in the combination setting while the CDK inhibitor, p21, was dramatically increased. Hypo-phosphorylation of retinoblastoma protein, directly linked to E2F complex inactivation, was also observed and consistent with reduced proliferation and the decreased frequency of S-phase cells observed in EDU incorporation assays. Taken together, these findings support a role for selective HDAC1/2i in combination with RA for the treatment of patients with high risk NB.

### Example 21

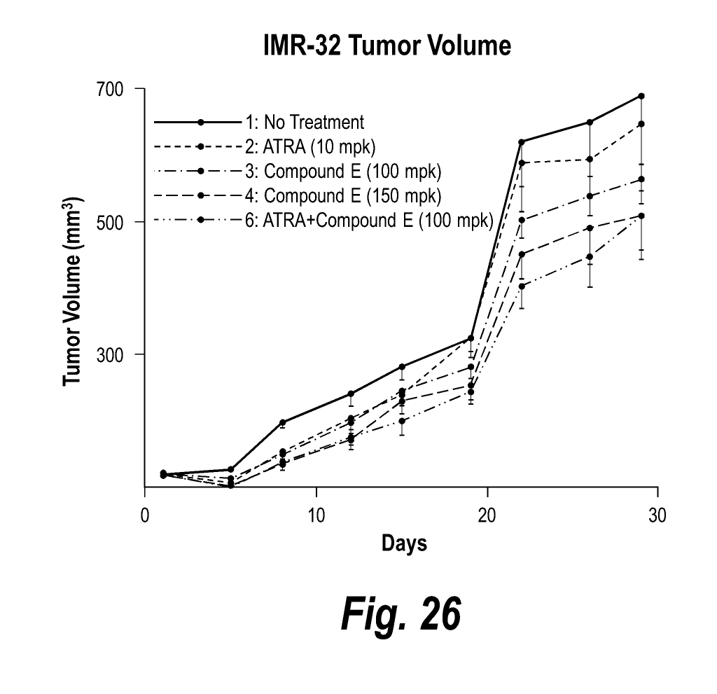

### HDAC1/2i Slows Neuroblastoma Tumor Growth (FIG. 26)

IMR-32 tumors were implanted in NUDE mice and Compound E and retinoic acid was administered orally once daily at the indicated doses on a 5/2 on/off schedule. Compound E treatment resulted in a dose-dependent trend toward reduced tumor growth, with an enhanced effect at the 100 mg/kg dosing group observed in combination with ATRA.

### Example 22

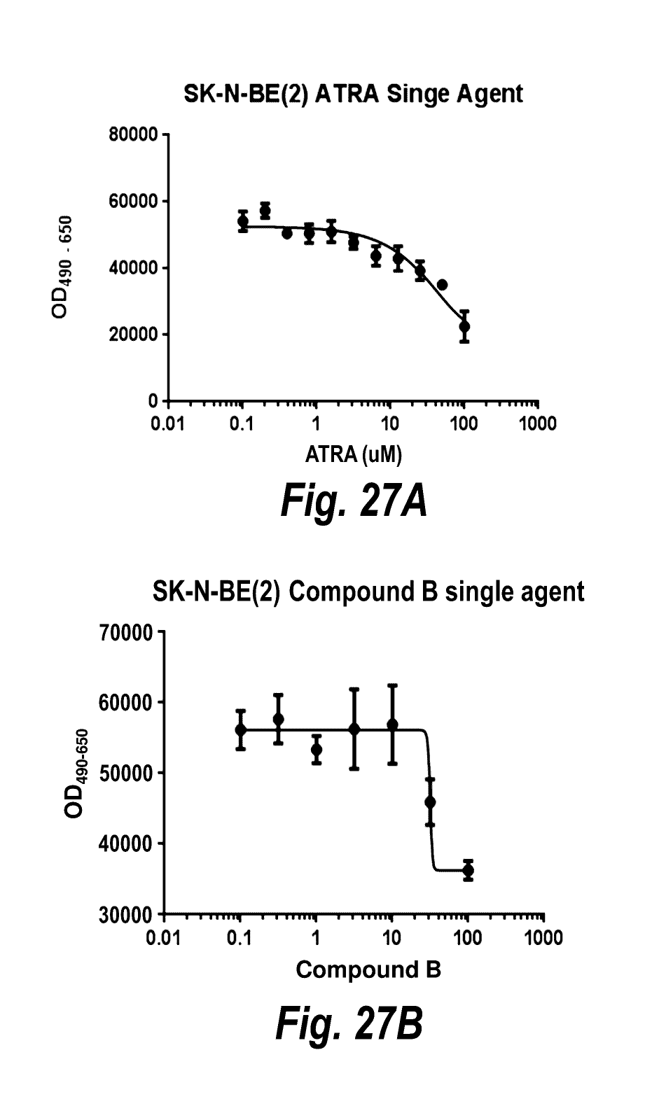

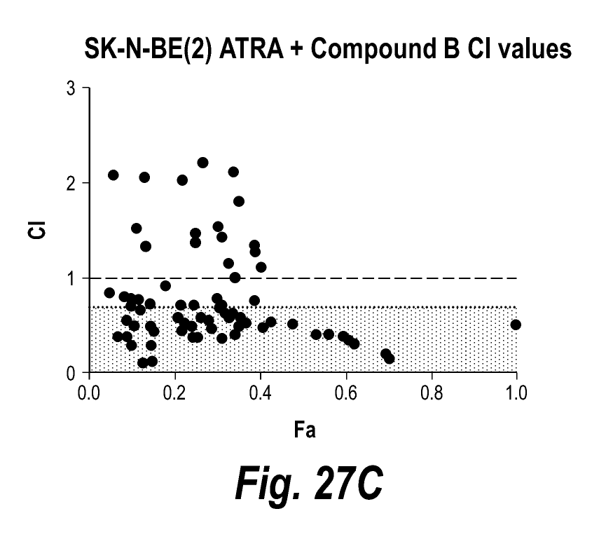

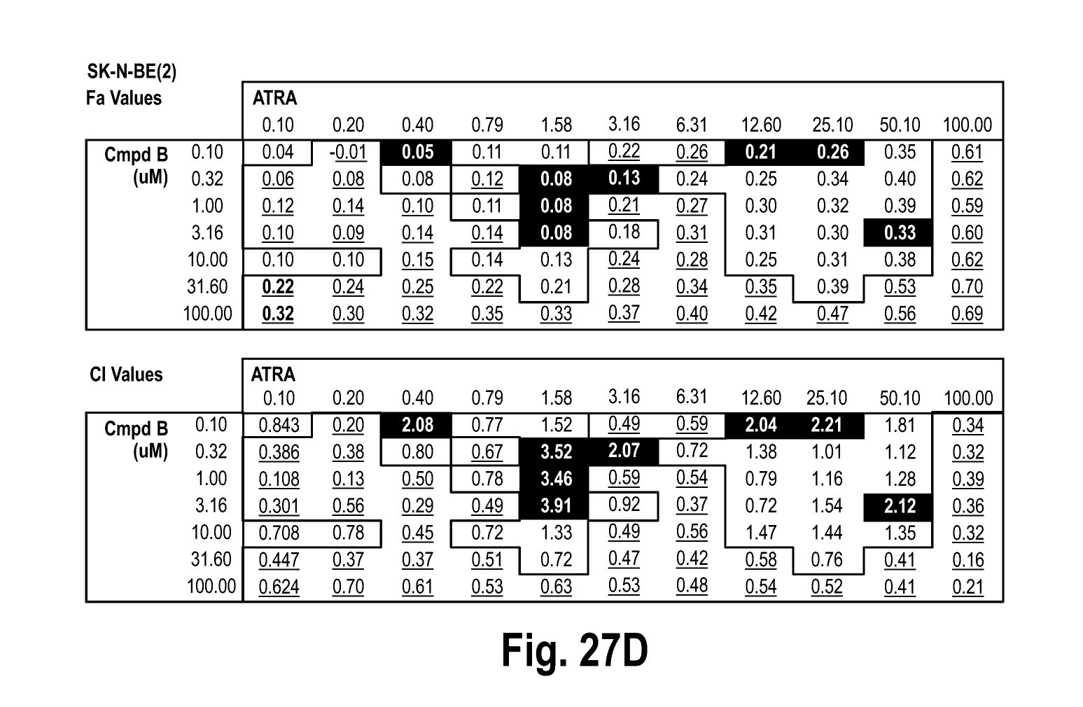

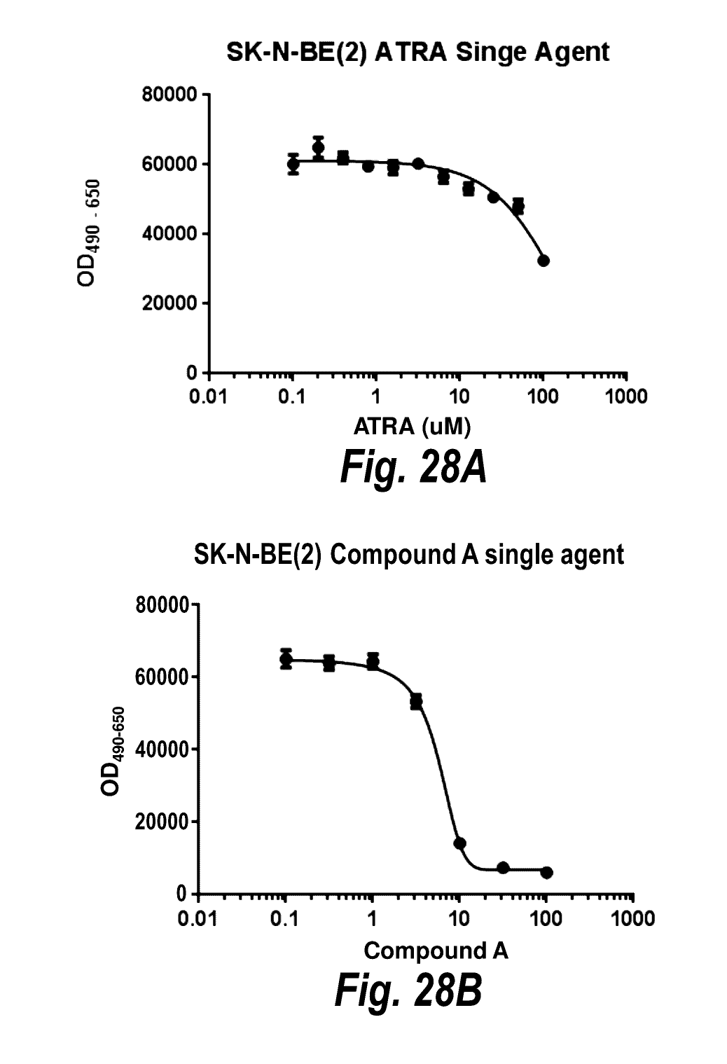

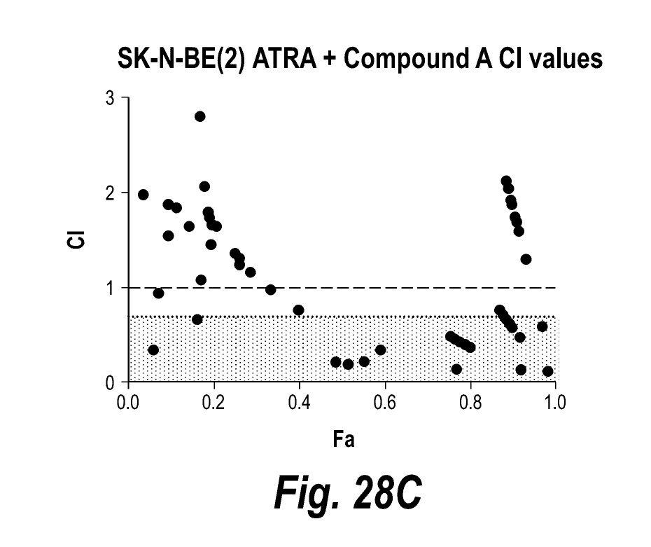

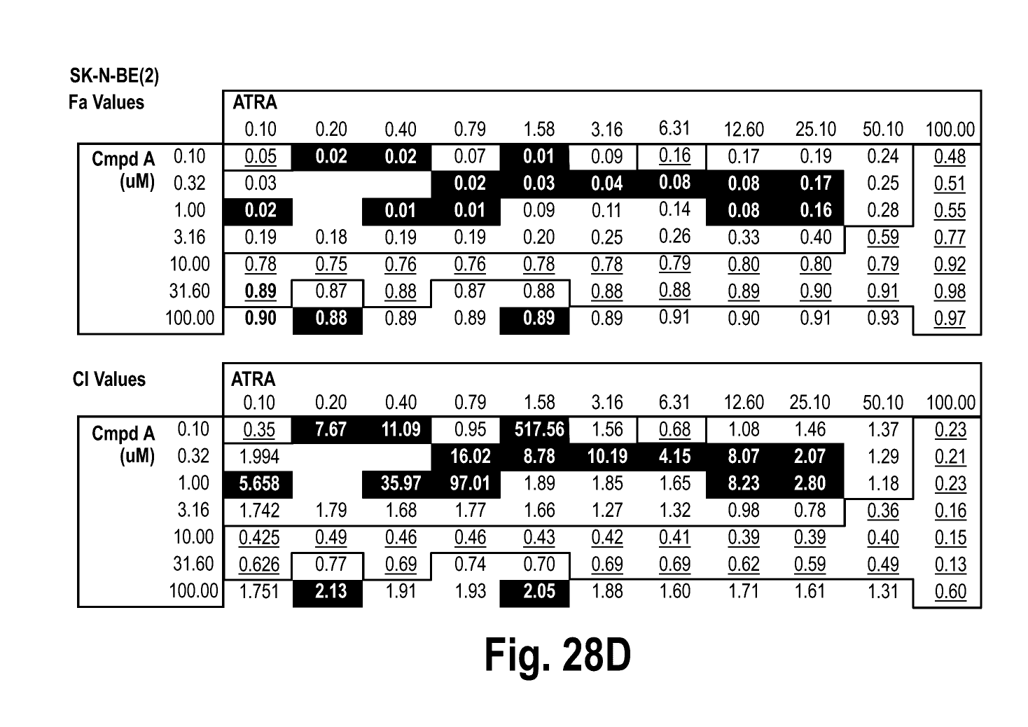

### Compound a and Compound B in Combination with ATRA Results in Synergistic Acute Toxicity Against Neuroblastoma Cells (FIGS. 27 and 28)

Neuroblastoma cells were treated with the indicated compounds, i.e., ATRA, Compound A, and Compound B, in a dose matrix. Viable cells were measured in an MTS assay and the combination index (CI) was calculated using the Chou-Talaay method. Any combinations where a CI value less than 1 is observed indicates a synergistic combination. These data illustrate the indicated compounds combine to induce synergistic neuroblastoma cell death. FIGS. 27A-D show the data obtained for Compound B plus ATRA and FIGS. 28A-D show the data obtained for Compound A plus ATRA.

### INCORPORATION BY REFERENCE

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

### EQUIVALENTS

Those skilled in the art will recognize, or be able to ascertain using no more than routine experimentation, many equivalents of the specific embodiments of the invention described herein. Such equivalents are intended to be encompassed by the following claims.

1-10. (canceled)

11. A method for treating neuroblastoma in a subject in need thereof comprising administering to the subject a therapeutically effective amount of a pharmaceutical combination comprising an HDAC inhibitor or a pharmaceutically acceptable salt thereof, and retinoic acid or a pharmaceutically acceptable salt thereof.

12. The method of claim 11, wherein the retinoic acid is ATRA.

13. The method of claim 11, wherein the HDAC inhibitor is an HDAC1/2 inhibitor.

14. The method of claim 11, wherein the HDAC inhibitor is a compound of Formula I: 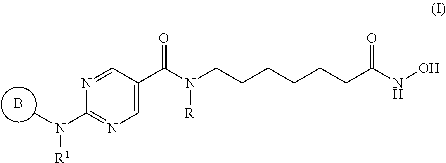
or a pharmaceutically acceptable salt thereof,
wherein,
ring B is aryl or heteroaryl;
R¹ is an aryl or heteroaryl, each of which may be optionally substituted by OH, halo, or C₁₋₆-alkyl; and
R is H or C₁₋₆-alkyl.

15. The method of claim 14, wherein R¹ is an aryl or heteroaryl, each of which is substituted by halo.

16. The method of claim 15, wherein the compound of Formula I is: 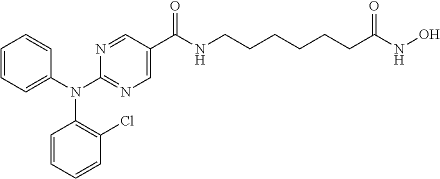 [SMILES: O=C(CCCCCCNC(=O)c1cnc(N(c2ccccc2)c2ccccc2Cl)nc1)NO]
or a pharmaceutically acceptable salt thereof.

17. The method of claim 13, wherein the HDAC1/2 inhibitor is an HDAC1/2-specific inhibitor.

18. The method of claim 17, wherein the HDAC1/2-specific inhibitor is a compound of Formula II: 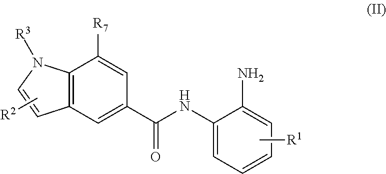
or a pharmaceutically acceptable salt thereof,
wherein,
R¹ is aryl or heteroaryl;
R² and R³ are each independently selected from C₃₋₆-cycloalkyl, C₁₋₆-alkyl-OR⁶, C₁₋₆-alkyl-C₃₋₆-cycloalkyl, C₁₋₆-alkyl-heterocycloalkyl, C₂₋₆-alkenyl;
R⁶ is H or C₁₋₆-alkyl; and
R⁷ is H or C₃₋₆-cycloalkyl.

19. The method of claim 18, wherein the compound of Formula II is: 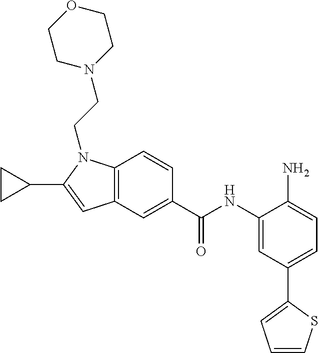 [SMILES: Nc1ccc(-c2cccs2)cc1NC(=O)c1ccc2c(c1)cc(C1CC1)n2CCN1CCOCC1]
or a pharmaceutically acceptable salt thereof.

20. The method of claim 11, wherein the HDAC inhibitor is: 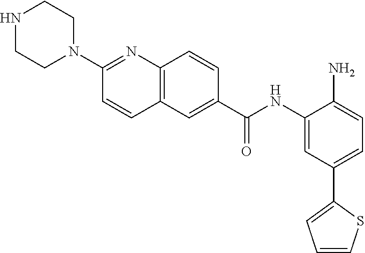 [SMILES: Nc1ccc(-c2cccs2)cc1NC(=O)c1ccc2nc(N3CCNCC3)ccc2c1]
or a pharmaceutically acceptable salt thereof.

21. The method of claim 11, wherein the subject was previously refractory to ATRA.

22-35. (canceled)

36. A method for treating neuroblastoma in a subject in need thereof comprising administering to the subject a therapeutically effective amount of an HDAC1/2 inhibitor inhibitor or a pharmaceutically acceptable salt thereof.

37-40. (canceled)

41. The method of claim 36, wherein the HDAC1/2 inhibitor is an HDAC1/2-specific inhibitor.

42. The method of claim 41, wherein the HDAC1/2-specific inhibitor is a compound of Formula II: 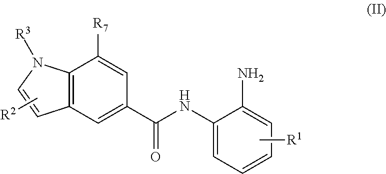
or a pharmaceutically acceptable salt thereof,
wherein,
R¹ is aryl or heteroaryl;
R² and R³ are each independently selected from C₃₋₆-cycloalkyl, C₁₋₆-alkyl-OR⁶, C₁₋₆-alkyl-C₃₋₆-cycloalkyl, C₁₋₆-alkyl-heterocycloalkyl, C₂₋₆-alkenyl;
R⁶ is H or C₁₋₆-alkyl; and
R⁷ is H or C₃₋₆-cycloalkyl.

43. The method of claim 42, wherein the compound of Formula II is: 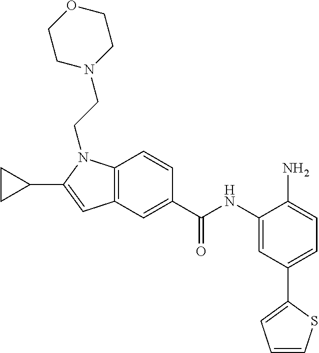 [SMILES: Nc1ccc(-c2cccs2)cc1NC(=O)c1ccc2c(c1)cc(C1CC1)n2CCN1CCOCC1]
or a pharmaceutically acceptable salt thereof.

44. The method of claim 36, wherein the HDAC1/2 inhibitor is: 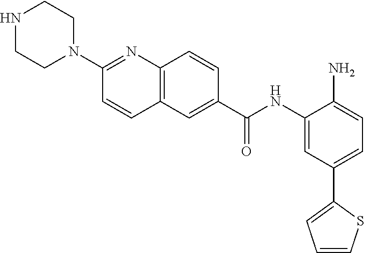 [SMILES: Nc1ccc(-c2cccs2)cc1NC(=O)c1ccc2nc(N3CCNCC3)ccc2c1]
or a pharmaceutically acceptable salt thereof.

45. The method of claim 36, further comprising administering to the subject a therapeutically effective amount of all-trans-retinoic acid or 13-cis-retinoic acid, or pharmaceutically acceptable salts thereof.
